SEQUENCE LISTING (24 sequences)

SEQ ID NO: 1 - protein, 54 aa, Pentadiplandra brazzeana
QDKCKKVYENYPVSKCQLANQCNYDCKLDKHARSGECFYDEKRNLQCICDYCEY

SEQ ID NO: 2 - protein, 53 aa, Pentadiplandra brazzeana
DKCKKVYENYPVSKCQLANQCNYDCKLDKHARSGECFYDEKRNLQCICDYCEY

SEQ ID NO: 3 - protein, 53 aa, Artificial Sequence
  Description of Artificial Sequence Synthetic polypeptide
DKCKKVYENYPVSKCQLKNQCNYDCKLDKHARSGECFYDEKRNLQCICDYCEY

SEQ ID NO: 4 - protein, 53 aa, Artificial Sequence
  Description of Artificial Sequence Synthetic polypeptide
DKCKKVYENYPVSKCQLANQCNYDCKLDKRARSGDCFYDAKRNLQCICDYCEY

SEQ ID NO: 5 - protein, 53 aa, Artificial Sequence
  Description of Artificial Sequence Synthetic polypeptide
DKCKKVYENYPVSKCQLKNQCNYDCKLDKRARSGDCFYDAKRNLQCICDYCEY

SEQ ID NO: 6 - protein, 207 aa, Thaumatococcus daniellii
ATFEIVNRCSYTVWAAASKGDAALDAGGRQLNSGESWTINVEPGTNGGKIWARTDCYFDDSGSGICKTGDCGGLLRCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 7 - protein, 207 aa, Thaumatococcus daniellii
ATFEIVNRCSYTVWAAASKGDAALDAGGRQLNSGESWTINVEPGTKGGKIWARTDCYFDDSGRGICRTGDCGGLLQCKRFGRPPTTLAEFSLNQYGKDYIDISNIKGFNVPMDFSPTTRGCRGVRCAADIVGQCPAKLKAPGGGCNDACTVFQTSEYCCTTGKCGPTEYSRFFKRLCPDAFSYVLDKPTTVTCPGSSNYRVTFCPTA

SEQ ID NO: 8 - protein, 45 aa, Dioscoreophyllum cumminsii
FREIKGYEYQLYVYASDKLFRADISEDYKTRGRKLLRFNGPVPPP

SEQ ID NO: 9 - protein, 50 aa, Dioscoreophyllum cumminsii
GEWEIIDIGPFTQNLGKFAVDEENKIGQYGRLTFNKVIRPCMKKTIYEEN

SEQ ID NO: 10 - protein, 32 aa, Capparis masaikai
EPLCRRQFQQHQHLRACQRYIRRRAQRGGLVD

SEQ ID NO: 11 - protein, 72 aa, Capparis masaikai
EQRGPALRLCCNQLRQVNKPCVCPVLRQAAHQQLYQGQIEGPRQVRQLFRAARNLPNICKIPAVGRCQFTRW

SEQ ID NO: 12 - protein, 33 aa, Capparis masaikai
QLWRCQRQFLQHQRLRACQRFIHRRAQFGGQPD

SEQ ID NO: 13 - protein, 72 aa, Capparis masaikai
QPRRPALRQCCNQLRQVDRPCVCPVLRQAAQQVLQRQIIQGPQQLRRLFDAARNLPNICNIPNIGACPFRAW

SEQ ID NO: 14 - protein, 32 aa, Capparis masaikai
EPLCRRQFQQHQHLRACQRYLRRRAQRGGLAD

SEQ ID NO: 15 - protein, 72 aa, Capparis masaikai
EQRGPALRLCCNQLRQVNKPCVCPVLRQAAHQQLYQGQIEGPRQVRRLFRAARNLPNICKIPAVGRCQFTRW

SEQ ID NO: 16 - protein, 28 aa, Capparis masaikai
EPLCRRQFQQHQHLRACQRYLRRRAQRG

SEQ ID NO: 17 - protein, 72 aa, Capparis masaikai; identical to SEQ ID NO: 15

SEQ ID NO: 18 - protein, 29 aa, Synsepalum dulcificum
MKELTMLSLSFFFVSALLAAAANPLLSAA

SEQ ID NO: 19 - protein, 191 aa, Synsepalum dulcificum
DSAPNPVLDIDGEKLRTGTNYYIVPVLRDHGGGLTVSATTPNGTFVCPPRVVQTRKEVDHDRPLAFFPENPKEDVVRVSTDLNINFSAFMPCRWTSSTVWRLDKYDESTGQYFVTIGGVKGNPGPETISSWFKIEEFCGSGFYKLVFCPTVCGSCKVKCGDVGIYIDQKGRRRLALSDKPFAFEFNKTVYF

SEQ ID NO: 20 - protein, 22 aa, Molineria latifolia
MAAKFLLTILVTFAAVASLGMA

SEQ ID NO: 21 - protein, 114 aa, Molineria latifolia
DNVLLSGQTLHADHSLQAGAYTLTIQNKCNLVKYQNGRQIWASNTDRRGSGCRLTLLSDGNLVIYDHNNNDVWGSACWGDNGKYALVLQKDGRFVIYGPVLWSLGPNGCRRVNG

SEQ ID NO: 22 - protein, 22 aa, Molineria latifolia
GITVAKDSTEPQHEDIKMVINN

SEQ ID NO: 23 - protein, 113 aa, Molineria latifolia
DSVLLSGQTLYAGHSLTSGSYTLTIQNNCNLVKYQHGRQIWASDTDGQGSQCRLTLRSDGNLIIYDDNNMVVWGSDCWGNNGTYALVLQQDGLFVIYGPVLWPLGLNGCRSLN

SEQ ID NO: 24 - protein, 23 aa, Molineria latifolia
GEITVAKDSTEPQHEDIKMVINN

(19) United States
(12) Patent Application Publication
(10) Pub. No.: US 2021/0051969 A1
(43) Pub. Date: Feb. 25, 2021

(54) RECOMBINANTLY EXPRESSED TASTE MODIFYING POLYPEPTIDES AND PREPARATIONS AND FORMULATIONS COMPRISING THE SAME

(71) Applicant: Joywell Foods, Inc., Davis, CA (US)

(72) Inventors: Benjamin Thomas Roche, San Francisco, CA (US); Li Zuo Taitano, West Sacramento, CA (US); Alan Jay Perlstein, Davis, CA (US); Jason Ryder, Oakland, CA (US)

(21) Appl. No.: 17/012,742

(22) Filed: Sep. 4, 2020

Related U.S. Application Data
(63) Continuation of application No. PCT/US2019/021053, filed on Mar. 6, 2019.
(60) Provisional application No. 62/640,491, filed on Mar. 8, 2018.
(60) Provisional application No. 62/639,363, filed on Mar. 6, 2018.

(51) Int. Cl. A23C 9/13; A23C 9/137

(52) U.S. Cl. CPC A23C 9/1322; A23C 9/137

(57) ABSTRACT

The present disclosure provides taste-modifying polypeptides and methods of producing the same. In some cases, the taste-modifying polypeptides may be produced recombinantly. In some cases, the taste-modifying polypeptides comprise a miraculin protein or a brazzein protein. The taste-modifying polypeptides may be provided in various formulations for human consumption.

### CROSS-REFERENCE

This application is a continuation application of International Application No. PCT/US2019/021053, filed Mar. 6, 2019, which claims the benefit of U.S. Provisional Application Nos. 62/639,363, filed Mar. 6, 2018, and 62/640,491, filed Mar. 8, 2018, which applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Sep. 3, 2020, is named 53394-701_301_SL.txt and is 16,903 bytes in size.

### BACKGROUND

Taste-modifying agents are useful in a variety of applications, including, but not limited to, human consumption. Taste-modifying agents, such as sweetening agents, may be useful for imparting a sweet taste to prepared foods, beverages, and medications ingested orally. Foods and beverages for general consumption that can be prepared with taste-modifying agents include, for example, baked goods, candies, and soft-drinks. Taste-modifying agents can also be formulated as part medications, in addition to products for general consumption. The use of taste-modifying agents, such as taste-modifying polypeptides, can serve various purposes, such as making the consumption of a product formulated with the taste-modifying polypeptide more enjoyable. In some cases, the use of taste-modifying agents may enhance the taste of foods for cancer patients having altered taste as a result of cancer treatment (e.g., chemotherapy, radiation therapy, etc).

Various sweeteners are available, including naturally occurring carbohydrates, such as those obtainable from plants, and artificial sweeteners. However, consumption of large quantities of natural and artificial sweeteners can be associated with various health risks, including diabetes and obesity.

### SUMMARY

In view of the foregoing, there is a need for alternative taste-modifying agents useful for a variety of applications, including, but not limited to, the preparation of foods, beverages, and medications. For example, alternative taste-modifying agents may have a sweeter taste than naturally occurring carbohydrates (e.g., saccharides) and artificial sweeteners, and, as a result, a lesser amount of the taste-modifying agent can be used to obtain a similar or comparable sweetness level. The present disclosure provides alternative taste-modifying agents such as taste-modifying polypeptides that can enhance a sweet taste. The present disclosure also provides methods of producing and purifying alternative taste-modifying agents.

Taste-modifying agents (e.g., taste-modifying polypeptides) can be formulated into a variety of products and may be consumed in order to achieve a desired level of taste-modification. In some cases, the taste-modifying polypeptide may yield a sweet taste in a subject (e.g., a human). The present disclosure provides taste-modifying polypeptides that can impart, elicit, and/or enhance a sweet taste, for example. The present disclosure also provides methods of purifying taste-modifying polypeptides from plant sources.

In one aspect, a recombinant cell is provided containing therein at least one copy of a stably integrated heterologous nucleic acid sequence encoding a taste-modifying protein or a functional fragment thereof. In some cases, the recombinant cell is a recombinant yeast cell. In some cases, the recombinant yeast cell is of the phylum Ascomycota. In some cases, the recombinant yeast cell is of the genus Komagataella or Kluyveromyces. In some cases, the recombinant yeast cell is of the species Pichia pastoris. In some cases, the recombinant yeast cell is of the species Kluyveromyces lactis. In some cases, the recombinant cell is a recombinant bacterial cell. In some cases, the recombinant bacterial cell is of the species Escherichia coli. In some cases, the recombinant cell is a filamentous fungal cell. In some cases, the recombinant cell is an insect cell. In some cases, the recombinant cell is a mammalian cell. In some cases, the taste-modifying protein is itself flavorless. In some cases, the taste-modifying protein is a taste enhancer. In some cases, the taste enhancer enhances a sweet taste in a subject. In some cases, the subject is a human. In some cases, the taste-modifying protein inhibits one or more bitter flavors. In some cases, the taste-modifying protein binds to one or more taste receptors on a tongue of a subject. In some cases, the one or more taste receptors comprise at least one type 1 sweet taste receptor (TAS1R). In some cases, the at least one type 1 sweet taste receptor is selected from the group consisting of: TAS1R1, TAS1R2, and TAS1R3. In some cases, the one or more taste receptors comprise at least one type 2 bitter taste receptor (TAS2R). In some cases, the at least one type 2 bitter taste receptor is selected from the group consisting of: TAS2R1, TAS2R50, and TAS2R60. In some cases, the heterologous nucleic acid sequence encodes a miraculin protein or a functional fragment thereof, a brazzein protein or a functional fragment thereof, a pentadin protein or a functional fragment thereof, a curculin protein or a functional fragment thereof, a monellin protein or a functional fragment thereof, a thaumatin protein or a functional fragment thereof, or a mabinlin protein or a functional fragment thereof. In some cases, the recombinant cell secretes the taste-modifying protein or a functional fragment thereof into a culture media. In some cases, the recombinant cell expresses the taste-modifying protein or a functional fragment thereof intracellularly. In some cases, the heterologous nucleic acid sequence is codon optimized for expression in the recombinant cell. In some cases, the heterologous nucleic acid sequence is operably linked to an inducible promoter or a constitutive promoter. In some cases, the inducible promoter comprises a nucleic acid sequence from an Aox1 promoter, an Aox2 promoter, or a Lac4 promoter. In some cases, the constitutive promoter is a GAP promoter.

In another aspect, a culture medium is provided comprising the recombinant cell of any of the preceding.

In another aspect, a recombinant cell is provided containing therein a heterologous nucleic acid sequence encoding a miraculin protein or a functional fragment thereof having at least 90% identity to SEQ ID NO: 19 and containing at least one substitution modification relative to SEQ ID NO: 19.

In yet another aspect, a recombinant cell is provided containing therein a heterologous nucleic acid sequence encoding a brazzein protein or a functional fragment thereof having at least 90% identity to any one of SEQ ID NOs: 1-5 and containing at least one substitution modification relative to SEQ ID NO: 1.

In yet another aspect, a recombinant cell is provided containing therein a heterologous nucleic acid sequence encoding a thaumatin protein or a functional fragment thereof having at least 90% identity to any one of SEQ ID NOs: 6 or 7 and containing at least one substitution modification relative to any one of SEQ ID NOs: 6 or 7.

In yet another aspect, a recombinant cell is provided containing therein a heterologous nucleic acid sequence encoding a curculin protein or a functional fragment thereof having at least 90% identity to any one of SEQ ID NOs: 21 or 23 and containing at least one substitution modification relative to any one of SEQ ID NOs: 21 or 23.

In yet another aspect, a recombinant cell is provided containing therein a heterologous nucleic acid sequence encoding a monellin protein or a functional fragment thereof having at least 90% identity to any one of SEQ ID NOs: 8 or 9 and containing at least one substitution modification relative to any one of SEQ ID NOs: 8 or 9.

In yet another aspect, a recombinant cell is provided containing therein a heterologous nucleic acid sequence encoding a mabinlin protein or a functional fragment thereof having at least 90% identity to any one of SEQ ID NOs: 10-17 and containing at least one substitution modification relative to any one of SEQ ID NOs: 10-17.

In yet another aspect, a recombinant cell is provided containing therein a heterologous nucleic acid sequence encoding a pentadin protein or a functional fragment thereof having at least 90% identity to a wild-type pentadin protein and containing at least one substitution modification as compared to a wild-type pentadin protein.

In another aspect, a polynucleotide is provided encoding a non-naturally occurring miraculin protein comprising a nucleotide sequence that encodes a miraculin protein or a functional fragment thereof having at least 90% identity to SEQ ID NO: 19.

In yet another aspect, a polynucleotide is provided encoding a non-naturally occurring brazzein protein comprising a nucleotide sequence that encodes a brazzein protein or a functional fragment thereof having at least 90% identity to any one of SEQ ID NOs: 1-5.

In yet another aspect, a polynucleotide is provided encoding a non-naturally occurring thaumatin protein comprising a nucleotide sequence that encodes a thaumatin protein or a functional fragment thereof having at least 90% identity to any one of SEQ ID NOs: 6 or 7.

In yet another aspect, a polynucleotide is provided encoding a non-naturally occurring curculin protein comprising a nucleotide sequence that encodes a curculin protein or a functional fragment thereof having at least 90% identity to any one of SEQ ID NOs: 21 or 23.

In yet another aspect, a polynucleotide is provided encoding a non-naturally occurring pentadin protein comprising a nucleotide sequence that encodes a pentadin protein or a functional fragment thereof.

In yet another aspect, a polynucleotide is provided encoding a non-naturally occurring monellin protein comprising a nucleotide sequence that encodes a monellin protein or a functional fragment thereof having at least 90% identity to any one of SEQ ID NOs: 8 or 9.

In yet another aspect, a polynucleotide is provided encoding a non-naturally occurring mabinlin protein comprising a nucleotide sequence that encodes a mabinlin protein or a functional fragment thereof having at least 90% identity to any one of SEQ ID NOs: 10-17.

In another aspect, a vector is provided comprising a polynucleotide according to any one of the preceding and a heterologous nucleic acid sequence. In another aspect, a cell is provided comprising the preceding vector.

In another aspect, a process is provided for isolating a heterologous taste-modifying protein or functional fragment thereof from a recombinant cell culture media, the process comprising: filtering a supernatant comprising the taste-modifying protein or functional fragment thereof from the recombinant cell culture media through a porous membrane, thereby isolating the taste-modifying protein or functional fragment thereof. In some cases, the process further comprises incubating a recombinant cell in cell culture media for a period of time. In some cases, the recombinant cell secretes the heterologous taste-modifying protein or functional fragment thereof into the supernatant. In some cases, the process further comprises, prior to the filtering, lysing the recombinant cell to release the taste-modifying protein or functional fragment thereof into the supernatant. In some cases, the taste-modifying protein or functional fragment thereof is a miraculin protein or a functional fragment thereof. In some cases, the taste-modifying protein or functional fragment thereof is a brazzein or a functional fragment thereof. In some cases, the taste modifying protein or functional fragment thereof is selected from the group consisting of: a pentadin protein or a functional fragment thereof, a curculin protein or a functional fragment thereof, a monellin protein or a functional fragment thereof, a thaumatin protein or a functional fragment thereof, and a mabinlin protein or a functional fragment thereof. In some cases, an amount of a supernatant contaminant is equal to or less than 1 part per million. In some cases, the porous membrane comprises pores ranging from about 0.01 μm to about 0.5 μm in diameter. In some cases, the process further comprises, adjusting a pH of the filtered supernatant to an acidic pH. In some cases, the process further comprises adjusting a pH of the filtered supernatant to a basic pH. In some cases, the recombinant cell is a recombinant yeast cell. In some cases, the recombinant yeast cell is of the phylum Ascomycota. In some cases, the recombinant yeast cell is of the genus Komagataella or Kluyveromyces. In some cases, the recombinant yeast cell is of the species Pichia pastoris. In some cases, the recombinant yeast cell is of the species Kluyveromyces lactis. In some cases, the recombinant cell is a recombinant bacterial cell. In some cases, the recombinant bacterial cell is of the species Escherichia coli. In some cases, the recombinant cell is a filamentous fungal cell. In some cases, the recombinant cell is an insect cell. In some cases, the recombinant cell is a mammalian cell.

In another aspect, an oral dosage form is provided comprising a recombinant taste-modifying polypeptide or a functional fragment thereof. In some cases, the oral dosage form comprises from about 0.00001 g to about 0.5 g of the recombinant taste-modifying polypeptide or a functional fragment thereof per unit dose. In some cases, the taste-modifying polypeptide or a functional fragment thereof is selected from the group consisting of: a miraculin protein or a functional fragment thereof, a brazzein protein or a functional fragment thereof, a pentadin protein or a functional fragment thereof, a curculin protein or a functional fragment thereof, a monellin protein or a functional fragment thereof, a thaumatin protein or a functional fragment thereof, or a mabinlin protein or a functional fragment thereof. In some cases, the taste-modifying protein or a functional fragment thereof is purified from a recombinant cell. In some cases, the taste-modifying protein or a functional fragment thereof is purified from a recombinant cell culture media. In some cases, the recombinant cell is a recombinant yeast cell. In some cases, the recombinant yeast cell is of the phylum Ascomycota. In some cases, the recombinant yeast cell is of the genus Komagataella or Kluyveromyces. In some cases, the recombinant yeast cell is of the species Pichia pastoris. In some cases, the recombinant yeast cell is of the species Kluyveromyces lactis. In some cases, the recombinant cell is a recombinant bacterial cell. In some cases, the recombinant bacterial cell is of the species Escherichia coli. In some cases, the recombinant cell is a filamentous fungal cell. In some cases, the recombinant cell is an insect cell. In some cases, the recombinant cell is a mammalian cell. In some cases, the recombinant taste-modifying polypeptide or a functional fragment thereof is a miraculin protein comprising a sequence that is has at least 80% sequence identity to SEQ ID NO: 19. In some cases, the recombinant taste-modifying polypeptide or a functional fragment thereof is a brazzein protein comprising a sequence that has at least 80% sequence identity to any one of SEQ ID NOs: 1-5. In some cases, the recombinant taste-modifying polypeptide or a functional fragment thereof is a pentadin protein comprising a sequence that has at least 80% sequence identity to a wild-type pentadin protein. In some cases, the recombinant taste-modifying polypeptide or a functional fragment thereof is a thaumatin protein comprising a sequence that has at least 80% sequence identity to any one of SEQ ID NOs: 6 or 7. In some cases, the recombinant taste-modifying polypeptide or a functional fragment thereof is a monellin protein comprising a sequence that has at least 80% sequence identity to any one of SEQ ID NOs: 8 or 9. In some cases, the recombinant taste-modifying polypeptide or a functional fragment thereof is a mabinlin protein comprising a sequence that has at least 80% sequence identity to any one of SEQ ID NOs: 10-17. In some cases, the recombinant taste-modifying polypeptide or a functional fragment thereof is a curculin protein comprising a sequence that has at least 80% sequence identity to any one of SEQ ID NOs: 21 or 23. In some cases, the oral dosage form further comprises an excipient. In some cases, the excipient comprises a pH stabilizer. In some cases, the oral dosage form is in the form of a film or a packet. In some cases, the oral dosage form is in the form of a powder. In some cases, the oral dosage form is in the form of an aerosol, vapor, spray, or mist. In some cases, the oral dosage form is packaged into a container selected from the group consisting of: a box, a tube, ajar, a vial, a bag, a drum, a bottle, and a can. In some cases, the container contains information describing directions for use. In some cases, a dissolution rate of the oral dosage form is more than about 80% within about a first 30 minutes following entry of the oral dosage form into a use environment. In some cases, the oral dosage form does not exhibit dose-proportionality. In some cases, the recombinant taste-modifying polypeptide or functional fragment thereof does not comprise xylose.

In another aspect, a composition formulated for consumption by a human subject is provided, the composition comprising a recombinant taste-modifying polypeptide or a functional fragment thereof. In some cases, the recombinant taste-modifying polypeptide or a functional fragment thereof is selected from the group consisting of: a miraculin protein or a functional fragment thereof, a brazzein protein or a functional fragment thereof, a pentadin protein or a functional fragment thereof, a curculin protein or a functional fragment thereof, a monellin protein or a functional fragment thereof, a thaumatin protein or a functional fragment thereof, or a mabinlin protein or a functional fragment thereof. In some cases, the recombinant taste-modifying protein or a functional fragment thereof is purified from a recombinant cell. In some cases, the recombinant taste-modifying protein or a functional fragment thereof is purified from a recombinant cell culture media. In some cases, the recombinant cell is a recombinant yeast cell. In some cases, the recombinant yeast cell is of the phylum Ascomycota. In some cases, the recombinant yeast cell is of the genus Komagataella or Kluyveromyces. In some cases, the recombinant yeast cell is of the species Pichia pastoris. In some cases, the recombinant yeast cell is of the species Kluyveromyces lactis. In some cases, the recombinant cell is a recombinant bacterial cell. In some cases, the recombinant bacterial cell is of the species Escherichia coli. In some cases, the recombinant cell is a filamentous fungal cell. In some cases, the recombinant cell is an insect cell. In some cases, the recombinant cell is a mammalian cell. In some cases, the recombinant taste-modifying polypeptide or a functional fragment thereof is a miraculin protein comprising a sequence that has at least 80% sequence identity to SEQ ID NO: 19. In some cases, the recombinant taste-modifying polypeptide or a functional fragment thereof is a brazzein protein comprising a sequence that has at least 80% sequence identity to any one of SEQ ID NOs: 1-5. In some cases, the recombinant taste-modifying polypeptide or a functional fragment thereof is a pentadin protein comprising a sequence that has at least 80% sequence identity to a wild-type pentadin protein. In some cases, the recombinant taste-modifying polypeptide or a functional fragment thereof is a thaumatin protein comprising a sequence that has at least 80% sequence identity to any one of SEQ ID NOs: 6 or 7. In some cases, the recombinant taste-modifying polypeptide or a functional fragment thereof is a monellin protein comprising a sequence that has at least 80% sequence identity to any one of SEQ ID NOs: 8 or 9. In some cases, the recombinant taste-modifying polypeptide or a functional fragment thereof is a mabinlin protein comprising a sequence that has at least 80% sequence identity to any one of SEQ ID NOs: 10-17. In some cases, the recombinant taste-modifying polypeptide or a functional fragment thereof is a curculin protein comprising a sequence that has at least 80% sequence identity to any one of SEQ ID NOs: 21 or 23. In some cases, the composition is formulated as an edible gel product. In some cases, the edible gel product is selected from the group consisting of: a gel, a jelly, a compote, a puree, a fruit bite, and a gummy candy. In some cases, the composition is formulated as a liquid beverage product. In some cases, the liquid beverage product is selected from the group consisting of: a soda product, a juice product, a tea product, a coffee product, a milk product, a water product, and an alcoholic beverage product. In some cases, the composition is formulated as a dairy product. In some cases, the dairy product is a yogurt product. In some cases, the yogurt product comprises: (i) yogurt; and (ii) the recombinant taste-modifying polypeptide or functional fragment thereof. In some cases, the composition comprises the recombinant taste-modifying polypeptide or functional fragment thereof in a separate formulation from the yogurt. In some cases, the recombinant taste-modifying polypeptide or functional fragment thereof is formulated as a gel, a jelly, a compote, or a puree. In some cases, the composition is formulated as a film. In some cases, the film is a coating. In some cases, the coating is a coating on a surface of a solid substrate. In some cases, the solid substrate is a utensil. In some cases, the utensil is selected from the group consisting: a straw, a spoon, a knife, and a fork. In some cases, the composition is formulated as a frozen food product. In some cases, the frozen food product is selected from the group consisting of: an ice cream product, a sorbet product, a frozen popsicle product, and a shaved ice product. In some cases, the frozen food product is a frozen popsicle product. In some cases, the frozen popsicle product comprises the recombinant taste-modifying polypeptide or functional fragment thereof as a coating on a surface of the frozen popsicle product. In some cases, the coating on a surface of the frozen popsicle product is a gel, a jelly, a compote, or a puree. In some cases, the coating on a surface of the frozen popsicle product covers about 1% to about 50% of the surface. In some cases, the composition does not comprise a saccharide. In some cases, the recombinant taste-modifying polypeptide or functional fragment thereof does not comprise xylose.

In another aspect, a composition is provided comprising: (i) less than about 5% by weight of a recombinant miraculin protein or fragment thereof; and (ii) additives at a concentration of at least about 95% by weight, wherein the additives are selected from the group consisting of: excipients, carriers, diluents, solubilizers, flavorants, preservatives, colorants, adjuvants, and any combination thereof.

In another aspect, a composition is provided comprising: (i) less than about 5% by weight of a recombinant brazzein protein or fragment thereof; and (ii) additives at a concentration of at least about 95% by weight, wherein the additives are selected from the group consisting of: excipients, carriers, diluents, solubilizers, flavorants, preservatives, colorants, and adjuvants.

In another aspect, a process is provided for purifying a protein from a plant extract comprising: (a) filtering the plant extract through a membrane whereby a hydrostatic pressure forces an amount of a liquid against a semipermeable membrane, thereby obtaining a first filtrate; and (b) filtering the first filtrate through a nickel affinity column or a cationic resin, thereby obtaining a purified protein from the plant extract. In another aspect, the protein comprises an affinity tag. In another aspect, the affinity tag is a histidine tag. In another aspect, the protein is a miraculin protein. In another aspect, the process further comprises, centrifuging the plant extract prior to filtering it. In some cases, the process further comprises, dialyzing the purified protein from the plant extract.

In yet another aspect, a composition is provided comprising: (i) yogurt; and (ii) a taste-modifying polypeptide. In some cases, the taste-modifying polypeptide is miraculin. In some cases, the miraculin is purified miraculin. In some cases, the purified miraculin is present in the composition in an amount from about 0.0001% w/w to about 0.0050% w/w. In some cases, the miraculin is contained within miracle berry powder. In some cases, the miracle berry powder is present in the composition in an amount from about 0.05% w/w to about 5% w/w. In some cases, the taste-modifying polypeptide is selected from the group consisting of: brazzein, curculin, monellin, mabinlin, thaumatin, and pentadin. In some cases, the taste-modifying polypeptide is a recombinant taste-modifying polypeptide.

In yet another aspect, a composition is provided comprising: a frozen popsicle comprising a taste-modifying polypeptide. In some cases, the taste-modifying polypeptide is miraculin. In some cases, the miraculin is purified miraculin. In some cases, the purified miraculin is present in the composition in an amount from about 0.0001% w/w to about 0.0050% w/w. In some cases, the miraculin is contained within miracle berry powder. In some cases, the miracle berry powder is present in the composition in an amount from about 0.1% w/w to about 5% w/w. In some cases, the taste-modifying polypeptide is selected from the group consisting of: brazzein, curculin, monellin, mabinlin, thaumatin, and pentadin. In some cases, the taste-modifying polypeptide is in a formulation coating the frozen popsicle. In some cases, the formulation is a gel, a jelly, a compote, or a puree. In some cases, the formulation coats about 1% to about 50% of a surface of the frozen popsicle. In some cases, the taste-modifying polypeptide is a recombinant taste-modifying polypeptide.

In yet another aspect, a composition is provided comprising: a gummy candy and a taste-modifying polypeptide. In some cases, the taste-modifying polypeptide is miraculin. In some cases, the miraculin is purified miraculin. In some cases, the purified miraculin is present in the composition in an amount from about 0.001% w/w to about 0.050% w/w. In some cases, the miraculin is contained within miracle berry powder. In some cases, the miracle berry powder is present in the composition in an amount from about 0.1% w/w to about 15% w/w. In some cases, the taste-modifying polypeptide is selected from the group consisting of: brazzein, curculin, monellin, mabinlin, thaumatin, and pentadin. In some cases, the taste-modifying polypeptide is a recombinant taste-modifying polypeptide.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Some novel features of the invention are set forth in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting example of expression of recombinant miraculin in Kluyveromyces lactis, as confirmed by SDS-PAGE/Western blotting.

FIG. 2 depicts a non-limiting example of gel filtration results after cation exchange purification of recombinant brazzein expressed in Kluyveromyces lactis.

FIG. 3 depicts a non-limiting example of purified recombinant brazzein from Kluyveromyces lactis as confirmed by SDS-PAGE/Western blotting.

FIG. 4 depicts a non-limiting example of protein identification verification by liquid chromatography tandem mass spectrometry (LC MS/MS) of recombinant brazzein (−1) expressed in Kluyveromyces lactis.

FIG. 5 depicts a non-limiting example of expression of recombinant miraculin in Pichia pastoris as confirmed by dot blot.

FIG. 6 depicts a non-limiting example of expression of recombinant brazzein in Pichia pastoris as confirmed by dot blot.

FIG. 7 depicts a non-limiting example of expression and purification of recombinant miraculin in Escherichia coli as confirmed by SDS-PAGE/Western blotting.

FIG. 8 depicts a non-limiting example of expression and purification of recombinant brazzein in Escherichia coli as confirmed by SDS-PAGE/Western blotting.

FIG. 9 depicts a side-by-side gustatory comparison of brazzein variants and the calculated equivalent concentration to a 4.6% sucrose solution.

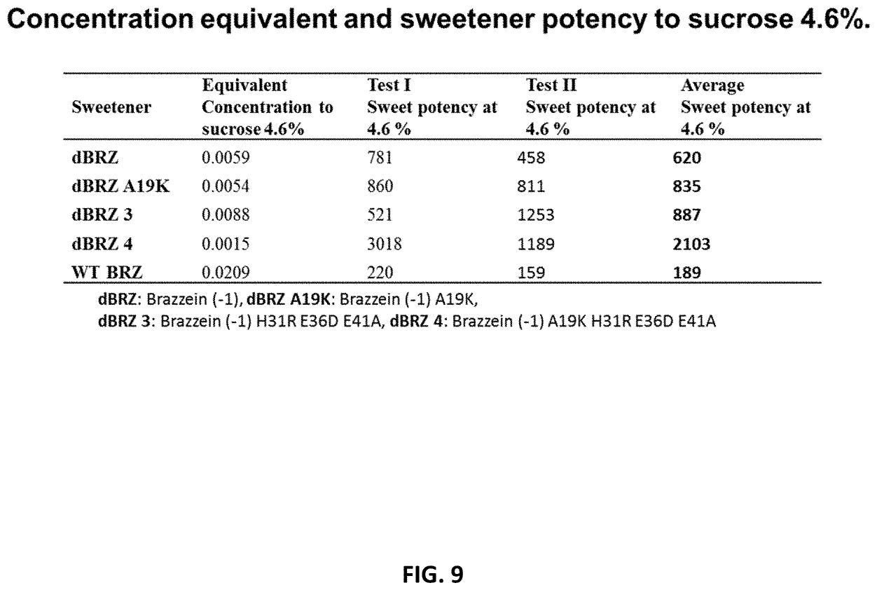

### DETAILED DESCRIPTION

The present disclosure provides taste-modifying agents comprising taste-modifying polypeptides capable of generating and/or modifying a taste. In some aspects, the taste-modifying agents may have a sweet taste. Taste-modifying agents disclosed herein can generate and/or modify a taste by enhancing, lessening, and/or altering a taste. In some aspects, the taste-modifying agents may generate and/or modify a sweet taste. In some aspects, the taste-modifying polypeptides themselves may be tasteless. In other aspects, the taste-modifying polypeptides themselves may have a sweet taste. The taste-modifying agents disclosed herein may be useful for a variety of applications, including, but not limited to, the preparation of foods, beverages, and medications. Methods and compositions for producing and purifying the taste-modifying agents are also disclosed.

Taste is a form of chemoreception which occurs in taste receptors in the mouth and is one of a variety of mechanisms used by mammals to sense stimuli and the external environment. Humans and other mammals have taste receptors on taste buds of the tongue and other areas, including the epiglottis. Each taste bud can have a pore that opens out to the surface of the tongue, enabling molecules and ions taken into the mouth to reach the receptor cells inside.

The taste sensations, which include salt, sour, sweet, bitter, and umami, can be sensed by one or more mechanism(s) and by one or more taste receptor(s). Taste receptors involved in the sensation of taste include Type 1 taste receptors (TAS1, e.g., TAS1R2 and TAS1R3) and Type 2 taste receptors (TAS2, e.g., TAS2R1-TAS2R50). Type 1 taste receptors are generally associated with sweetness whereas Type 2 taste receptors are generally associated with bitterness. Combinations of these receptors in dimers or other complexes can also contribute to different perceptions of taste.

In some aspects, taste-modifying polypeptides disclosed herein may generate a taste, such as a salty, sour, sweet, bitter, or umami taste. In some embodiments, a taste-modifying polypeptide disclosed herein may generate a salty taste. In some embodiments, a taste-modifying polypeptide disclosed herein may generate a sour taste. In some embodiments, a taste-modifying polypeptide disclosed herein may generate a sweet taste. In some embodiments, a taste-modifying polypeptide disclosed herein may generate a bitter taste. In some embodiments, a taste-modifying polypeptide disclosed herein may generate an umami taste.

In some aspects, taste-modifying polypeptides disclosed herein can modify a taste, such as a salty, sour, sweet, bitter, or umami taste. In some cases, taste-modifying polypeptides disclosed herein can modify a taste, such as enhance a salty, sour, sweet, bitter, or umami taste. In some cases, taste-modifying polypeptides disclosed herein can modify a taste, such as lessen a salty, sour, sweet, bitter, or umami taste. In some cases, taste-modifying polypeptides disclosed herein can modify a taste, such as alter a salty, sour, sweet, bitter, or umami taste. In some cases, taste-modifying polypeptides disclosed herein may alter a salty taste to one of a sour, sweet, bitter, or umami taste. In some cases, taste-modifying polypeptides disclosed herein may alter a sour taste to one of a salty, sweet, bitter, or umami taste. In some cases, taste-modifying polypeptides disclosed herein may alter a sweet taste to one of a salty, sour, bitter, or umami taste. In some cases, taste-modifying polypeptides disclosed herein may alter a bitter taste to one of a salty, sour, sweet, or umami taste. In some cases, taste-modifying polypeptides disclosed herein may alter an umami taste to one of a salty, sour, sweet, or bitter taste.

The terms “about” and “approximately,” as used herein, means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean a range of up to 20%, up to 10%, up to 5%, or up to 1% of a given value. For example, about can mean up to ±20%, ±19%, ±18%, ±17%, ±16%, ±15%, ±14%, ±13%, ±12%, ±11%, ±10%, ±9%, ±8%, ±7%, ±6%, ±5%, ±4%, ±3%, ±2%, or ±1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein and in the appended claims, the singular forms “a,” “an,” and “the” include plural reference unless the context clearly indicates otherwise.

### I. Taste-Modifying Polypeptides

In an aspect, the present disclosure provides taste-modifying polypeptides, such as those that may generate a sweet taste and/or modify a taste in humans. In some cases, taste-modifying polypeptides of the disclosure may be sweet-tasting and may taste sweet, for example, to a human subject. In some cases, taste-modifying polypeptides of the disclosure may alter a taste to yield a sweet-taste. The taste which is altered to a sweet-taste may be a sweet taste or a non-sweet taste. The disclosed polypeptides can generate a sweet taste and/or enhance a sweet taste of a composition, for example, a food. The terms “polypeptide,” “peptide,” and “protein” are used interchangeably herein to refer to polymers of amino acids of any length. The polymer may be linear or branched, it may comprise modified amino acids, and/or it may be interrupted by non-amino acids. The terms also encompass an amino acid polymer that has been modified, for example, disulfide bond formation, glycosylation, lipidation, acetylation, phosphorylation, or any other manipulation, such as conjugation with a labeling component. As used herein, the term “amino acid” refers to either natural and/or unnatural or synthetic amino acids, including glycine and both the D or L optical isomers, and amino acid analogs and peptidomimetics.

Taste-modifying polypeptides of the disclosure can be isolated from various naturally occurring sources, such as plants and the fruits thereof. Taste-modifying polypeptides of the disclosure can be expressed recombinantly in protein expression systems. In some cases, the sweetness properties and taste-modifying properties of naturally occurring polypeptides can be enhanced by protein engineering techniques. In some instances, the sweetness properties and taste-modifying properties of naturally occurring polypeptides can be enhanced by mutating no more than 1 amino acid, no more than 2 amino acids, no more than 3 amino acids, no more than 4 amino acids, no more than 5 amino acids, no more than 6 amino acids, no more than 7 amino acids, no more than 8 amino acids, no more than 9 amino acids, or no more than 10 amino acids, as compared to a wild-type sequence of a taste-modifying protein derived from a plant.

Taste-modifying polypeptides provided herein may have a sweeter taste than naturally occurring carbohydrates (e.g., saccharides) and artificial sweeteners. A non-limiting example of a taste-modifying polypeptide having a sweet taste includes the protein brazzein, which can be found in the West African fruit of the climbing plant Oubli (Pentadiplandra brazzeana). In some cases, the sweetness of the protein, as determined by a human subject, for example in a side-by-side gustatory comparison, can be greater than that of carbohydrate sweeteners and artificial sweeteners.

In some aspects, the taste modifying polypeptide of the disclosure is a brazzein protein, a functional fragment thereof, or a mutant thereof. Brazzein is a protein having 54 amino acid residues (Table 1) and a molecular weight of approximately 6.5 kiloDaltons (kDa). Based on structural analysis, brazzein appears to have four evenly spaced disulfide bonds and no sulfhydryl groups, one alpha-helix and three strands of anti-parallel beta sheet. Residues 29-33, 36, and 39-43, as well as the C-terminus of the protein, may be involved in the sweet taste of the protein. The charge of the protein may also play an important role in its interaction with the sweet taste receptor.

TABLE 1
Brazzein and Brazzein Variants

| Brazzein Variant | Amino Acid Sequence | Sweetness |
|---|---|---|
| wild-type Brazzein | QDKCKKVYENYPVSKCQLANQCNYDCKLDK | — |
| | HARSGECFYDEKRNLQCICDYCEY (SEQ ID | |
| | NO: 1) | |
| Brazzein (−1) | DKCKKVYENYPVSKCQLANQCNYDCKLDKH | 3X sweeter than |
| | ARSGECFYDEKRNLQCICDYCEY (SEQ ID NO: | wild-type |
| | 2) | |
| Brazzein (−1) A19K | DKCKKVYENYPVSKCQLKNQCNYDCKLDKH | 4X sweeter than |
| | ARSGECFYDEKRNLQCICDYCEY (SEQ ID NO: | wild-type |
| | 3) | |
| Brazzein (−1) H31R | DKCKKVYENYPVSKCQLANQCNYDCKLDKR | 4.7X sweeter than |
| E36D E41A | ARSGDCFYDAKRNLQCICDYCEY (SEQ ID | wild-type |
| | NO: 4) | |
| Brazzein (−1) A19K | DKCKKVYENYPVSKCQLKNQCNYDCKLDKR | 11X sweeter than |
| H31R E36D E41A | ARSGDCFYDAKRNLQCICDYCEY (SEQ ID | wild-type |
| | NO: 5) | |

On a weight basis, brazzein may taste 500 to 2000 times sweeter than sucrose, as compared to a 10% sucrose and a 2% sucrose solution, respectively. Sucrose, a commonly used sweetener, is a disaccharide molecule composed of glucose and fructose.

The present disclosure also provides brazzein mutants having a sweeter taste compared to wild-type brazzein. In some cases, a brazzein mutant is perceived to taste, for example by a human subject in side-by-side gustatory comparison, at least twice as sweet as wild-type brazzein (e.g., at least 2×, 3×, 4×, 5×, 6×, 7×, 8×, 9×, 10×, 100×, 1000×, or at least 10,000× as sweet as wild-type brazzein).

A brazzein mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of brazzein in Table 1. The terms “percent (%) identity” and “percent (%) identical,” as used herein with reference to polypeptide sequences, refer to the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways, for instance, using publicly available computer software such as BLAST, BLAST-2, ALIGN or Megalign (DNASTAR) software. Percent identity may be measured over the length of an entire defined polypeptide sequence, for example, a wild-type protein sequence or sequence as defined by a particular SEQ ID number, or may be measured over a shorter length, for example, over the length of a fragment taken from a larger, defined polypeptide sequence.

A brazzein mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of brazzein shown in Table 1. A brazzein mutant can have at least one (e.g., at least 2, 3, 4, 5, 6, 7, 8, 9, 10 or more) insertions, deletions, and/or substitutions as compared to the wild-type sequence of brazzein shown in Table 1.

In some instances, a brazzein mutant may contain at least one (e.g., at least 2, 3, 4, 5, 6, 7, 8, 9, 10 or more) amino acid insertion(s) compared to the wild-type protein. A brazzein mutant of the present disclosure can have at least one amino acid insertion (e.g., at least 2, 3, 4, 5, 6, 7, 8, 9, 10 or more amino acid insertions) at the N-terminus, the C-terminus, or in an inner region of the protein. In some cases, at least two inserted amino acids may be consecutive amino acids (e.g., amino acids adjacent to one another). In some cases, at least two inserted amino acids may be non-consecutive amino acids. In some cases, the amino acid insertion(s) may occur in a region of the protein that interacts with a taste receptor.

In some instances, a brazzein mutant may contain at least one (e.g., at least 2, 3, 4, 5, 6, 7, 8, 9, 10 or more) amino acid deletion(s) compared to the wild-type protein. A brazzein mutant of the present disclosure can have at least one amino acid deletion (e.g., at least 2, 3, 4, 5, 6, 7, 8, 9, 10 or more amino acid deletions) at the N-terminus, the C-terminus, or in an inner region of the protein. In some cases, at least two deleted amino acids may be consecutive amino acids (e.g., amino acids adjacent to one another). In some cases, at least two deleted amino acids may be non-consecutive amino acids. In some cases, the amino acid deletion(s) may occur in a region of the protein that interacts with a taste receptor. In an example, a brazzein mutant contemplated herein may have an amino acid deletion at the N-terminus of the protein (e.g., Table 1, Brazzein (˜1)). Brazzein(˜1), when compared to wild-type brazzein, can be perceived to taste twice as sweet as wild-type brazzein.

In some cases, a brazzein mutant of the present disclosure can have at least one (e.g., at least 2, 3, 4, 5, 6, 7, 8, 9, 10 or more) amino acid substitution(s) compared to wild-type brazzein. In some cases, the amino acid substitution may occur in a region of the protein that interacts with a taste receptor. In some cases, the substitution may enhance the interaction between brazzein and the taste receptor. In some cases, the substitution may decrease the interaction between brazzein and the taste receptor. In some cases, a brazzein mutant may be mutated at at least one amino acid residue selected from amino acid (aa) positions aa 19, aa 29, aa 30, aa 31, aa 32, aa 33, aa 36, aa 39, aa 40, aa 41, aa 42, aa 43, aa 50, and aa 54 of the wild-type sequence. The wild-type amino acid residue at any one of the aforementioned positions can be substituted with any suitable amino acid to enhance or increase a sweet-taste of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) as compared to the wild-type amino acid (e.g., non-conservative substitution). In some cases, a brazzein mutant useful as a taste-modifying polypeptide comprises at least one amino acid substitution selected from the group consisting of: A19K, D29A, D29K, D29N, H31R, E36D, D40A, D40K, E41A, D50K, and Y54W. In some cases, a brazzein mutant useful as a taste-modifying polypeptide comprises at least two amino acid substitutions selected from the group consisting of: A19K, D29A, D29K, D29N, H31R, E36D, D40A, D40K, E41A, D50K, and Y54W. In some cases, a brazzein mutant useful as a taste-modifying polypeptide comprises at least three amino acid substitutions selected from the group consisting of: A19K, D29A, D29K, D29N, H31R, E36D, D40A, D40K, E41A, D50K, and Y54W. In some cases, a brazzein mutant useful as a taste-modifying polypeptide comprises at least four amino acid substitutions selected from the group consisting of; A19K, D29A, D29K, D29N, H31R, E36D, D40A, D40K, E41A, D50K, and Y54W. In some cases, a brazzein mutant useful as a taste-modifying polypeptide comprises at least five amino acid substitutions selected from the group consisting of: A19K, D29A, D29K, D29N, H31R, E36D, D40A, D40K, E41A, D50K, and Y54W. In some cases, a brazzein mutant useful as a taste-modifying polypeptide comprises at least six amino acid substitutions selected from the group consisting of: A19K, D29A, D29K, D29N, H31R, E36D, D40A, D40K, E41A, D50K, and Y54W. In some cases, a brazzein mutant useful as a taste-modifying polypeptide comprises at least seven amino acid substitutions selected from the group consisting of: A19K, D29A, D29K, D29N, H31R, E36D, D40A, D40K, E41A, D50K, and Y54W. In some cases, a brazzein mutant useful as a taste-modifying polypeptide comprises at least eight amino acid substitutions selected from the group consisting of: A19K, D29A, D29K, D29N, H31R, E36D, D40A, D40K, E41A, D50K, and Y54W. In a non-limiting example, a brazzein mutant can have an amino acid deletion at the N-terminus of the protein and an A19K substitution (Table 1, Brazzein (˜1) A19K). Such a mutant can have a perceived sweetness that is at least four-times sweeter than wild-type brazzein. In another non-limiting example, a brazzein mutant can have an amino acid deletion at the N-terminus of the protein and A19K, H31R, E36D, and E41A substitutions (Table 1, Brazzein (˜1) A19K H31R E36D E41A). Such a mutant can have a perceived sweetness that is at least eleven-times sweeter than wild-type brazzein.

A brazzein mutant of the present disclosure may have any combination of the insertions, deletions, and substitutions described herein. A taste-modifying polypeptide comprising brazzein (e.g., wild-type, fragment thereof, or mutant thereof) may bind to a sweet taste receptor with an appropriate dissociation constant.

In some aspects, the taste-modifying polypeptide of the disclosure may be pentadin.

Pentadin can also be found in the fruit of the climbing plant Oubli and may also be used as a sweet-tasting polypeptide in embodiments herein. On a weight basis, pentadin may taste 500 times sweeter than sucrose.

In some aspects, the taste-modifying polypeptide of the disclosure may be a pentadin mutant. In some cases, a pentadin mutant may be perceived to taste, for example, by a human subject in side-by-side gustatory comparison, at least twice as sweet as wild-type pentadin (e.g., at least 2×, 3×, 4×, 5×, 6×, 7×, 8×, 9×, 10×, 100×, 1000×, or at least 10,000× as sweet as wild-type pentadin). A pentadin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of pentadin. A pentadin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of pentadin. A pentadin mutant can have at least one (e.g., at least 2, 3, 4, 5, 6, 7, 8, 9, 10 or more) amino acid insertions, deletions, and/or substitutions as compared to the wild-type sequence of pentadin. A pentadin mutant can have any combination of amino acid insertions, deletions, and substitutions relative to a wild-type sequence.

In some instances, a pentadin mutant may contain at least one (e.g., at least 2, 3, 4, 5, 6, 7, 8, 9, 10 or more) amino acid insertion(s) compared to the wild-type protein. A pentadin mutant of the present disclosure can have at least one amino acid insertion (e.g., at least 2, 3, 4, 5, 6, 7, 8, 9, 10 or more amino acid insertions) at the N-terminus, the C-terminus, or in an inner region of the protein. In some cases, at least two inserted amino acids may be consecutive amino acids (e.g., amino acids adjacent to one another). In some cases, at least two inserted amino acids may be non-consecutive amino acids. In some cases, the amino acid insertion(s) occurs in a region of the protein that interacts with a taste receptor.

In some instances, a pentadin mutant may contain at least one (e.g., at least 2, 3, 4, 5, 6, 7, 8, 9, 10 or more) amino acid deletion(s) compared to the wild-type protein. A pentadin mutant of the present disclosure can have at least one amino acid deletion (e.g., at least 2, 3, 4, 5, 6, 7, 8, 9, 10 or more amino acid deletions) at the N-terminus, the C-terminus, or in an inner region of the protein. In some cases, at least two deleted amino acids may be consecutive amino acids (e.g., amino acids adjacent to one another). In some cases, at least two deleted amino acids may be non-consecutive amino acids. In some cases, the amino acid deletion(s) occurs in a region of the protein that interacts with a taste receptor.

In some instances, a pentadin mutant of the present disclosure can have at least one (e.g., at least 2, 3, 4, 5, 6, 7, 8, 9, 10 or more) amino acid substitution(s) compared to wild-type pentadin. A pentadin mutant of the present disclosure can have at least one amino acid substitution (e.g., at least 2, 3, 4, 5, 6, 7, 8, 9, 10 or more amino acid substitution) at the N-terminus, the C-terminus, or in an inner region of the protein. In some cases, the amino acid substitution may occur in a region of the protein that interacts with a taste receptor. In some cases, the substitution may enhance the interaction between pentadin and the taste receptor. In some cases, the substitution may decrease the interaction between pentadin and the taste receptor. The wild-type amino acid residue can be substituted with any suitable amino acid to enhance or increase a sweet-taste of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) compared to the wild-type amino acid (e.g., non-conservative substitution).

In some aspects, the taste-modifying polypeptide of the disclosure is a thaumatin. Thaumatin refers to a class of sweet-tasting proteins isolated from the fruit of the tropical plant Thaumatococcus danielli. Two forms of thaumatin (thaumatin I and thaumatin II, Table 2), have been previously isolated. Thaumatin I is a protein of approximately 207 amino acids and has eight intramolecular disulfide bonds and contains no free cysteine residues. On a molar basis, thaumatin I may taste 10000 times sweeter than commonly used sugars.

TABLE 2
Thaumatin polypeptides

| | Amino Acid Sequence |
|---|---|
| Thaumatin I | ATFEIVNRCSYTVWAAASKGDAALDAGGRQL |
| | NSGESWTINVEPGTNGGKIWARTDCYFDDSGS |
| | GICKTGDCGGLLRCKRFGRPPTTLAEFSLNQY |
| | GKDYIDISNIKGFNVPMDFSPTTRGCRGVRCA |
| | ADIVGQCPAKLKAPGGGCNDACTVFQTSEYC |
| | CTTGKCGPTEYSRFFKRLCPDAFSYVLDKPTT |
| | VTCPGSSNYRVTFCPTA (SEQ ID NO: 6) |
| Thaumatin II | ATFEIVNRCSYTVWAAASKGDAALDAGGRQL |
| | NSGESWTINVEPGTKGGKIWARTDCYFDDSGR |
| | GICRTGDCGGLLQCKRFGRPPTTLAEFSLNQY |
| | GKDYIDISNIKGFNVPMDFSPTTRGCRGVRCA |
| | ADIVGQCPAKLKAPGGGCNDACTVFQTSEYC |
| | CTTGKCGPTEYSRFFKRLCPDAFSYVLDKPTT |
| | VTCPGSSNYRVTFCPTA (SEQ ID NO: 7) |

In some cases, the taste-modifying polypeptide of the disclosure is a thaumatin mutant (e.g., a thaumatin I mutant or a thaumatin II mutant). In some cases, a thaumatin mutant (e.g., a thaumatin I mutant or a thaumatin II mutant) is perceived to taste, for example, by a human subject in side-by-side gustatory comparison, at least twice as sweet as the wild-type protein (e.g., at least 2×, 3×, 4×, 5×, 6×, 7×, 8×, 9×, 10×, 100×, 1000×, or at least 10,000× as sweet as wild-type thaumatin I or wild-type thaumatin II). A thaumatin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of the protein. A thaumatin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of the protein. A thaumatin mutant can have at least one (e.g., at least 2, 3, 4, 5, 6, 7, 8, 9, 10 or more) amino acid insertions, deletions and/or substitutions as compared to the wild-type sequence of thaumatin. A thaumatin mutant can have any combination of amino acid insertions, deletions, and substitutions relative to a wild-type sequence.

In some instances, a thaumatin mutant may contain at least one (e.g., at least 2, 3, 4, 5, 6, 7, 8, 9, 10 or more) amino acid insertion(s) as compared to the wild-type protein. A thaumatin mutant of the present disclosure can have at least one amino acid insertion (e.g., at least 2, 3, 4, 5, 6, 7, 8, 9, 10 or more amino acid insertions) at the N-terminus, the C-terminus, or in an inner region of the protein. In some cases, at least two inserted amino acids may be consecutive amino acids (e.g., amino acids adjacent to one another). In some cases, at least two inserted amino acids may be non-consecutive amino acids. In some cases, the amino acid insertion(s) occurs in a region of the protein that interacts with a taste receptor.

In some instances, a thaumatin mutant may contain at least one (e.g., at least 2, 3, 4, 5, 6, 7, 8, 9, 10 or more) amino acid deletion(s) compared to the wild-type protein. A thaumatin mutant of the present disclosure can have at least one amino acid deletion (e.g., at least 2, 3, 4, 5, 6, 7, 8, 9, 10 or more amino acid deletions) at the N-terminus, the C-terminus, or in an inner region of the protein. In some cases, at least two deleted amino acids may be consecutive amino acids (e.g., amino acids adjacent to one another). In some cases, at least two deleted amino acids may be non-consecutive amino acids. In some cases, the amino acid deletion(s) may occur in a region of the protein that interacts with a taste receptor.

In some cases, a thaumatin mutant of the present disclosure can have at least one (e.g., at least 2, 3, 4, 5, 6, 7, 8, 9, 10 or more) amino acid substitution(s) compared to the wild-type protein. A thaumatin mutant of the present disclosure can have at least one amino acid substitution (e.g., at least 2, 3, 4, 5, 6, 7, 8, 9, 10 or more amino acid substitution) at the N-terminus, the C-terminus, or in an inner region of the protein. In some cases, the amino acid substitution may occur in a region of the protein that interacts with a taste receptor. In some cases, the substitution may enhance the interaction between thaumatin and the taste receptor. In some cases, the substitution may decrease the interaction between thaumatin and the taste receptor. The wild-type amino acid residue can be substituted with any suitable amino acid to enhance or increase a sweet-taste of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) compared to the wild-type amino acid (e.g., non-conservative substitution).

A taste-modifying polypeptide comprising thaumatin I (e.g., wild-type, fragment thereof, or mutant thereof) may bind to a sweet taste receptor with an appropriate dissociation constant. A taste-modifying polypeptide comprising thaumatin II (e.g., wild-type, fragment thereof, or mutant thereof) may bind to a sweet taste receptor with an appropriate dissociation constant.

In some cases, the taste-modifying polypeptide of the disclosure is a monellin. Monellin refers to a sweet protein originally discovered in the fruit of the West African shrub Dioscoreophyllum cumminsii. Monellin consists of two non-covalently associated polypeptide chains, an A chain of 44 amino acid residues and a B chain of 50 amino acid residues (Table 3). On a molar basis, monellin may taste 100,000 times sweeter than commonly used sugars. On a weight basis, monellin may taste several thousand times sweeter that commonly used sugars.

TABLE 3
Monellin Polypeptides

| | Amino Acid Sequence |
|---|---|
| Monellin chain A | FREIKGYEYQLYVYASDKLFRADISEDYKTRG |
| (44 amino acids) | RKLLRFNGPVPPP (SEQ ID NO: 8) |
| Monellin chain B | GEWEIIDIGPFTQNLGKFAVDEENKIGQYGRLT |
| (50 amino acids) | FNKVIRPCMKKTIYEEN (SEQ ID NO: 9) |

In some instances, the taste-modifying polypeptide of the disclosure may be a monellin mutant. A monellin mutant may be mutated in chain A, chain B, or in both chains. In some cases, a monellin mutant may be perceived to taste, for example, by a human subject in side-by-side gustatory comparison, at least twice as sweet as the wild-type protein (e.g., at least 2×, 3×, 4×, 5×, 6×, 7×, 8×, 9×, 10×, 100×, 1000×, or at least 10,000× as sweet as wild-type monellin). A monellin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of the protein. A monellin mutant provided herein can have a mutated chain A that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of chain A. A monellin mutant provided herein can have a mutated chain B that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of chain B. A monellin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of the protein. A monellin mutant provided herein can have a mutated chain A that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of chain A. A monellin mutant provided herein can have a mutated chain B that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of chain B. A monellin mutant can have at least one (e.g., at least 2, 3, 4, 5, 6, 7, 8, 9, 10 or more) amino acid insertions, deletions and/or substitution as compared to the wild-type sequence of monellin. A monellin mutant can have any combination of amino acid insertions, deletions, and substitutions relative to a wild-type sequence.

In some instances, a monellin mutant (e.g., chain A, chain B, or both chains) may contain at least one (e.g., at least 2, 3, 4, 5, 6, 7, 8, 9, 10 or more) amino acid insertion(s) compared to the wild-type protein. A monellin mutant of the present disclosure can have at least one amino acid insertion (e.g., at least 2, 3, 4, 5, 6, 7, 8, 9, 10 or more amino acid insertions) at the N-terminus, the C-terminus, or in an inner region of chain A, chain B, or both chains of monellin. In some cases, at least two inserted amino acids may be consecutive amino acids (e.g., amino acids adjacent to one another). In some cases, at least two inserted amino acids may be non-consecutive amino acids. In some cases, the amino acid insertion(s) may occur in a region of the protein that interacts with a taste receptor.

In some instances, a monellin mutant (e.g., chain A, chain B, or both chains) may contain at least one (e.g., at least 2, 3, 4, 5, 6, 7, 8, 9, 10 or more) amino acid deletion(s) compared to the wild-type protein. A monellin mutant of the present disclosure can have at least one amino acid deletion (e.g., at least 2, 3, 4, 5, 6, 7, 8, 9, 10 or more amino acid deletions) at the N-terminus, the C-terminus, or in an inner region of chain A, chain B, or both chains of monellin. In some cases, at least two deleted amino acids may be consecutive amino acids (e.g., amino acids adjacent to one another). In some cases, at least two deleted amino acids may be non-consecutive amino acids. In some cases, the amino acid deletion(s) occurs in a region of the protein that interacts with a taste receptor.

In some cases, a monellin mutant (e.g., chain A, chain B, or both chains) of the present disclosure can have at least one (e.g., at least 2, 3, 4, 5, 6, 7, 8, 9, 10 or more) amino acid substitution(s) compared to the wild-type protein. A monellin mutant of the present disclosure can have at least one amino acid substitution (e.g., at least 2, 3, 4, 5, 6, 7, 8, 9, 10 or more amino acid deletions) at the N-terminus, the C-terminus, or in an inner region of chain A, chain B, or both chains of monellin. In some cases, the amino acid substitution may occur in a region of the protein that interacts with a taste receptor. In some cases, the substitution may enhance the interaction between monellin and the taste receptor. In some cases, the substitution may decrease the interaction between monellin and the taste receptor. The wild-type amino acid residue can be substituted with any suitable amino acid to enhance or increase a sweet-taste of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) as compared to the wild-type amino acid (e.g., non-conservative substitution).

A taste-modifying polypeptide comprising monellin (e.g., wild-type, fragment thereof, or mutant thereof) may bind to a sweet taste receptor with an appropriate dissociation constant.

In some instances, the taste-modifying polypeptide of the disclosure may be mabinlin. Mabinlin is a sweet protein derived from Capparis masaikai. Mabinlin may be any one of mabinlin-1, mabinlin-2, mabinlin-3, or mabinlin-4. Mabinlin-1 may consist of an A chain composed of 32 amino acid residues and a B chain composed of 72 amino acid residues (Table 4, SEQ ID NOs: 10 and 11). Mabinlin-2 may consist of an A chain composed of 33 amino acid residues and a B chain composed of 72 amino acid residues (Table 4, SEQ ID NOs: 12 and 13). Mabinlin-3 may consist of an A chain composed of 32 amino acid residues and a B chain composed of 72 amino acid residues (Table 4, SEQ ID NOs: 14 and 15). Mabinlin-4 may consist of an A chain composed of 28 amino acid residues and a B chain composed of 72 amino acid residues (Table 4, SEQ ID NOs: 16 and 17). The B chain of a mabinlin protein may contain two intramolecular disulfide bonds and may be connected to the A chain through two intermolecular disulfide bridges. On a weight basis, the sweetness of a mabinlin protein may be perceived to be around 400 times that of sucrose.

TABLE 4
Mabinlin Polypeptides

| | Amino Acid Sequence |
|---|---|
| Mabinlin-1 chain A | EPLCRRQFQQHQHLRACQRYIRRRAQRGGLV |
| (32 amino acids) | D (SEQ ID NO: 10) |
| Mabinlin-1 chain B | EQRGPALRLCCNQLRQVNKPCVCPVLRQAAH |
| (72 amino acids) | QQLYQGQIEGPRQVRQLFRAARNLPNICK |
| | IPAVGRCQFTRW (SEQ ID NO: 11) |
| Mabinlin-2 chain A | QLWRCQRQFLQHQRLRACQRFIHRRAQFGGQ |
| (33 amino acids) | PD (SEQ ID NO: 12) |
| Mabinlin-2 chain B | QPRRPALRQCCNQLRQVDRPCVCPVLRQAAQ |
| (72 amino acids) | QVLQRQIIQGPQQLRRLFDAARNLPNICN |
| | IPNIGACPFRAW (SEQ ID NO: 13) |
| Mabinlin-3 chain A | EPLCRRQFQQHQHLRACQRYLRRRAQRGGLA |
| (32 amino acids) | D (SEQ ID NO: 14) |
| Mabinlin-3 chain B | EQRGPALRLCCNQLRQVNKPCVCPVLRQAAH |
| (72 amino acids) | QQLYQGQIEGPRQVRRLFRAARNLPNICK |
| | IPAVGRCQFTRW (SEQ ID NO: 15) |
| Mabinlin-4 chain A | EPLCRRQFQQHQHLRACQRYLRRRAQRG |
| (28 amino acids) | (SEQ ID NO: 16) |
| Mabinlin-4 chain B | EQRGPALRLCCNQLRQVNKPCVCPVLRQAAH |
| (72 amino acids) | QQLYQGQIEGPRQVRRLFRAARNLPNICK |
| | IPAVGRCQFTRW (SEQ ID NO: 17) |

In some instances, the taste-modifying polypeptide of the disclosure is a mabinlin mutant. For example, the taste-modifying polypeptide of the disclosure may be a mutated mabinlin-1, a mutated mabinlin-2, a mutated mabinlin-3, or a mutated mabinlin-4. A mabinlin mutant may be mutated in chain A, chain B, or both chains. In some cases, a mabinlin mutant may be perceived to taste, for example, by a human subject in side-by-side gustatory comparison, at least twice as sweet as the wild-type protein (e.g., at least 2×, 3×, 4×, 5×, 6×, 7×, 8×, 9×, 10×, 100×, 1000×, or at least 10,000× as sweet as a wild-type mabinlin protein). A mabinlin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of a mabinlin protein. A mabinlin mutant provided herein can have a mutated chain A that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of chain A. A mabinlin mutant provided herein can have a mutated chain B that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of chain B. A mabinlin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of a mabinlin protein. A mabinlin mutant provided herein can have a mutated chain A that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of chain A. A mabinlin mutant provided herein can have a mutated chain B that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of chain B. A mabinlin mutant can have at least one (e.g., at least 2, 3, 4, 5, 6, 7, 8, 9, 10 or more) amino acid insertions, deletions and/or substitutions as compared to the wild-type sequence of a mabinlin protein. A mabinlin mutant can have any combination of amino acid insertions, deletions, and substitutions relative to a wild-type sequence.

In some instances, a mabinlin mutant (e.g., chain A, chain B, or both chains) may contain at least one (e.g., at least 2, 3, 4, 5, 6, 7, 8, 9, 10 or more) amino acid insertion(s) compared to the wild-type protein. A mabinlin mutant of the present disclosure can have at least one amino acid insertion (e.g., at least 2, 3, 4, 5, 6, 7, 8, 9, 10 or more amino acid insertions) at the N-terminus, the C-terminus, or in an inner region of chain A, chain B, or both chains of a mabinlin protein. In some cases, at least two inserted amino acids may be consecutive amino acids (e.g., amino acids adjacent to one another). In some cases, at least two inserted amino acids may be non-consecutive amino acids. In some cases, the amino acid insertion(s) may occur in a region of the protein that interacts with a taste receptor.

In some instances, a mabinlin mutant (e.g., chain A, chain B, or both chains) may contain at least one (e.g., at least 2, 3, 4, 5, 6, 7, 8, 9, 10 or more) amino acid deletion(s) compared to the wild-type protein. A mabinlin mutant of the present disclosure can have at least one amino acid deletion (e.g., at least 2, 3, 4, 5, 6, 7, 8, 9, 10 or more amino acid deletions) at the N-terminus, the C-terminus, or in an inner region of chain A, chain B, or both chains of a mabinlin protein. In some cases, at least two deleted amino acids may be consecutive amino acids (e.g., amino acids adjacent to one another). In some cases, at least two deleted amino acids may be non-consecutive amino acids. In some cases, the amino acid deletion(s) may occur in a region of the protein that interacts with a taste receptor.

In some instances, a mabinlin mutant (e.g., chain A, chain B, or both chains) of the present disclosure can have at least one (e.g., at least 2, 3, 4, 5, 6, 7, 8, 9, 10 or more) amino acid substitution(s) compared to the wild-type protein. A mabinlin mutant of the present disclosure can have at least one amino acid substitution (e.g., at least 2, 3, 4, 5, 6, 7, 8, 9, 10 or more amino acid deletions) at the N-terminus, the C-terminus, or in an inner region of chain A, chain B, or both chains of a mabinlin protein. In some cases, the amino acid substitution may occur in a region of the protein that interacts with a taste receptor. In some cases, the substitution may enhance the interaction between mabinlin and the taste receptor. In some cases, the substitution may decrease the interaction between mabinlin and the taste receptor. The wild-type amino acid residue can be substituted with any suitable amino acid to enhance or increase a sweet-taste of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) compared to the wild-type amino acid (e.g., non-conservative substitution).

A taste-modifying polypeptide comprising mabinlin-1 (e.g., wild-type, fragment thereof, or mutant thereof) may bind to a sweet taste receptor with an appropriate dissociation constant. A taste-modifying polypeptide comprising mabinlin-2 (e.g., wild-type, fragment thereof, or mutant thereof) may bind to a sweet taste receptor with an appropriate dissociation constant. A taste-modifying polypeptide comprising mabinlin-3 (e.g., wild-type, fragment thereof, or mutant thereof) may bind to a sweet taste receptor with an appropriate dissociation constant. A taste-modifying polypeptide comprising mabinlin-4 (e.g., wild-type, fragment thereof, or mutant thereof) may bind to a sweet taste receptor with an appropriate dissociation constant.

In some instances, the taste-modifying polypeptide of the present disclosure may comprise a taste-altering polypeptide. Taste-altering polypeptides can themselves, in some cases, have a sweet taste. In some cases, taste-altering polypeptides can elicit a sweet taste without themselves having a sweet taste. Taste-altering polypeptides can interact with a taste receptor, such as TAS1R2 (also referred to as T1R2) and TAS1R3 (also referred to as T1R3). In some cases, taste-altering polypeptides may elicit a sweet taste from a sour taste. In some cases, taste-altering polypeptides alter a sour taste to yield a sweet taste. Non-limiting examples of taste-altering polypeptides capable of yielding a sweet taste from a composition, such as a food or a beverage, include the proteins miraculin and curculin.

In some instances, the taste-modifying polypeptide is miraculin or any variant thereof. Miraculin is a glycoprotein extracted from the fruit of Synsepalum dulcificum. Wild-type miraculin is approximately 191 amino acids in length (Table 5) and has carbohydrate chains. The molecular weight of the glycoprotein is 24.6 kDa, including 3.4 kDa (13.9% of the weight) of sugar constituted (on molar ratio) of glucosamine (31%), mannose (30%), fucose (22%), xylose (10%) and galactose (7%). Miraculin may occur as a tetramer (˜98.4 kDa), or a pair of dimers. Within each dimer, two miraculin glycoproteins may be linked by a disulfide bridge.

TABLE 5
Amino Acid Sequence of Miraculin

| | Amino Acid Sequence |
|---|---|
| SIGNAL (29) | MKELTMLSLS FFFVSALLAA AANPLLSAA |
| | (SEQ ID NO: 18) |
| Miraculin | DSAPNPVLDIDGEKLRTGTNYYIVPVLRDHGGGLTVSA |
| | TTPNGTFVCPPRVVQTRKEVDHDRPLAFFPENPKEDVV |
| | RVSTDLNINFSAFMPCRWTSSTVWRLDKYDESTGQYFV |
| | TIGGVKGNPGPETISSWFKIEEFCGSGFYKLVFCPTVC |
| | GSCKVKCGDVGIYIDQKGRRRLALSDKPFAFEFNKTVY |
| | F (SEQ ID NO: 19) |

Both tetramer miraculin and native dimer miraculin in its crude state can have a taste-modifying activity of altering sour tastes to yield sweet tastes. Miraculin may not be sweet by itself, but it can change the perception of sour to sweet. The duration and intensity of the taste-modifying phenomena can depend on various factors, including miraculin concentration, duration of contact of miraculin with the tongue, and acid concentration.

The present disclosure also provides miraculin mutants which may alter a taste to a greater extent and/or for a longer period of time as compared to wild-type miraculin. In some cases, a miraculin mutant may modify a taste, for example, a taste in a human subject, for a period of time at least twice as long as the wild-type protein (e.g., at least 2×, 3×, 4×, 5×, 6×, 7×, 8×, 9×, or 10× as long as the wild-type protein). In some cases, a miraculin mutant may be perceived to taste, for example, by a human subject in side-by-side gustatory comparison, at least twice as sweet as the wild-type protein (e.g., at least 2×, 3×, 4×, 5×, 6×, 7×, 8×, 9×, 10×, 100×, 1000×, or at least 10,000× as sweet as wild-type miraculin).

In some instances, the taste-modifying polypeptide of the disclosure may be a miraculin mutant. A miraculin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of the protein. A miraculin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of the protein. A miraculin mutant can have at least one (e.g., at least 2, 3, 4, 5, 6, 7, 8, 9, 10 or more) amino acid insertions, deletions and/or substitutions as compared to the wild-type sequence of miraculin. A miraculin mutant can have any combination of amino acid insertions, deletions, and substitutions relative to a wild-type sequence.

In some instances, a miraculin mutant may contain at least one (e.g., at least 2, 3, 4, 5, 6, 7, 8, 9, 10 or more) amino acid insertion(s) compared to the wild-type protein. A miraculin mutant of the present disclosure can have at least one amino acid insertion (e.g., at least 2, 3, 4, 5, 6, 7, 8, 9, 10 or more amino acid insertions) at the N-terminus, the C-terminus, or in an inner region of the protein. In some cases, at least two inserted amino acids may be consecutive amino acids (e.g., amino acids adjacent to one another). In some cases, at least two inserted amino acids may be non-consecutive amino acids. In some cases, the amino acid insertion(s) may occur in a region of the protein that interacts with a taste receptor.

In some instances, a miraculin mutant may contain at least one (e.g., at least 2, 3, 4, 5, 6, 7, 8, 9, 10 or more) amino acid deletion(s) compared to the wild-type protein. A miraculin mutant of the present disclosure can have at least one amino acid deletion (e.g., at least 2, 3, 4, 5, 6, 7, 8, 9, 10 or more amino acid deletions) at the N-terminus, the C-terminus, or in an inner region of the protein. In some cases, at least two deleted amino acids may be consecutive amino acids (e.g., amino acids adjacent to one another). In some cases, at least two deleted amino acids may be non-consecutive amino acids. In some cases, the amino acid deletion(s) may occur in a region of the protein that interacts with a taste receptor.

In some instances, a miraculin mutant of the present disclosure can have at least one (e.g., at least 2, 3, 4, 5, 6, 7, 8, 9, 10 or more) amino acid substitution(s) compared to the wild-type protein. A miraculin mutant of the present disclosure can have at least one amino acid substitution (e.g., at least 2, 3, 4, 5, 6, 7, 8, 9, 10 or more amino acid deletions) at the N-terminus, the C-terminus, or in an inner region of the protein. In some cases, the amino acid substitution may occur in a region of the protein that interacts with a taste receptor. In some cases, the substitution may enhance the interaction between miraculin and the taste receptor. In some cases, the substitution may decrease the interaction between miraculin and the taste receptor. The wild-type amino acid residue can be substituted with any suitable amino acid to enhance or increase the taste-modifying activity of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) compared to the wild-type amino acid (e.g., non-conservative substitution).

A taste-modifying polypeptide comprising miraculin (e.g., wild-type, fragment thereof, or mutant thereof) may bind to a sweet taste receptor with an appropriate dissociation constant.

In some instances, the taste-modifying polypeptide may be curculin or any variant thereof. Curculin is a protein capable of modifying taste and which also exhibits a sweet taste (e.g., sweet-tasting). Curculin can be isolated from the fruit of Curculigo latifolia (Hypoxidaceae). The active form of curculin is a heterodimer consisting of two monomeric units connected through two disulfide bridges. The mature monomers each consist of a sequence of 114 and 113 amino acids (Table 6), weighing approximately 12.5 kDa (curculin 1 or curculin A) and 12.7 kDa (curculin 2 or curculin B), respectively. While each of the two isoforms is capable of forming a homodimer, these generally do not possess the sweet taste nor the taste-modifying activity of the heterodimeric form. The heterodimeric form is sometimes referred to as “neoculin”. Curculin is considered to be a high-intensity sweetener, with a reported relative sweetness that is 430-2070 times sweeter than sucrose on a weight basis.

TABLE 6
Amino Acid Sequences of Curculin A and Curculin B

| | Amino Acid Sequence |
|---|---|
| SIGNAL (22) | MAAKFLLTILVTFAAVASLGMA (SEQ ID NO: 20) |
| Curculin A | DNVLLSGQTLHADHSLQAGAYTLTIQNKCNLVKYQNGR |
| | QIWASNTDRRGSGCRLTLLSDGNLVIYDHNNNDVWGSA |
| | CWGDNGKYALVLQKDGRFVIYGPVLWSLGPNGCRRVNG |
| | (SEQ ID NO: 21) |
| PROPEP (22) | GITVAKDSTEPQHEDIKMVINN (SEQ ID NO: 22) |
| SIGNAL (22) | MAAKFLLTILVTFAAVASLGMA (SEQ ID NO: 20) |
| Curculin B | DSVLLSGQTLYAGHSLTSGSYTLTIQNNCNLVKYQHGR |
| | QIWASDTDGQGSQCRLTLRSDGNLIIYDDNNMVVWGSD |
| | CWGNNGTYALVLQQDGLFVIYGPVLWPLGLNGCRSLN |
| | (SEQ ID NO: 23) |
| PROPEP (23) | GEITVAKDSTEPQHEDIKMVINN (SEQ ID NO: 24) |

In some cases, a curculin mutant may modify a taste, for example, a taste in a human subject, for a period of time at least twice as long as the wild-type protein (e.g., at least 3×, 4×, 5×, 6×, 7×, 8×, 9×, or 10× as long as the wild-type protein). In some cases, a curculin mutant may be perceived to taste, for example, by a human subject in side-by-side gustatory comparison, at least twice as sweet as the wild-type protein (e.g., at least 2×, 3×, 4×, 5×, 6×, 7×, 8×, 9×, 10×, 100×, 1000×, or at least 10,000× as sweet as wild-type curculin).

In some instances, the taste-modifying polypeptide of the disclosure may be a curculin mutant. A curculin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% identical to the wild-type sequence of the protein. A curculin mutant provided herein can be any non-naturally occurring protein that is at least 80%, 81%, 82%, 83%, 84%, 85%, 86%, 87%, 88%, 89%, 90%, 91%, 92%, 93%, 94%, 95%, 96%, 97%, 98%, or at least 99% homologous to the wild-type sequence of the protein. A curculin mutant can have at least one (e.g., at least 2, 3, 4, 5, 6, 7, 8, 9, 10 or more) amino acid insertions, deletions and/or substitutions as compared to the wild-type sequence of curculin. A curculin mutant can have any combination of amino acid insertions, deletions, and substitutions relative to a wild-type sequence.

In some instances, a curculin mutant may contain at least one (e.g., at least 2, 3, 4, 5, 6, 7, 8, 9, 10 or more) amino acid insertion(s) compared to the wild-type protein. A curculin mutant of the present disclosure can have at least one amino acid insertion (e.g., at least 2, 3, 4, 5, 6, 7, 8, 9, 10 or more amino acid insertions) at the N-terminus, the C-terminus, or in an inner region of the protein. In some cases, at least two inserted amino acids may be consecutive amino acids (e.g., amino acids adjacent to one another). In some cases, at least two inserted amino acids may be non-consecutive amino acids. In some cases, the amino acid insertion(s) may occur in a region of the protein that interacts with a taste receptor.

In some instances, a curculin mutant contains at least one (e.g., at least 2, 3, 4, 5, 6, 7, 8, 9, 10 or more) amino acid deletion(s) compared to the wild-type protein. A curculin mutant of the present disclosure can have at least one amino acid deletion (e.g., at least 2, 3, 4, 5, 6, 7, 8, 9, 10 or more amino acid deletions) at the N-terminus, the C-terminus, or in an inner region of the protein. In some cases, at least two deleted amino acids may be consecutive amino acids (e.g., amino acids adjacent to one another). In some cases, at least two deleted amino acids may be non-consecutive amino acids. In some cases, the amino acid deletion(s) may occur in a region of the protein that interacts with a taste receptor.

In some instances, a curculin mutant of the present disclosure can have at least one (e.g., at least 2, 3, 4, 5, 6, 7, 8, 9, 10 or more) amino acid substitution(s) compared to the wild-type protein. A curculin mutant of the present disclosure can have at least one amino acid substitution (e.g., at least 2, 3, 4, 5, 6, 7, 8, 9, 10 or more amino acid deletions) at the N-terminus, the C-terminus, or in an inner region of the protein. In some cases, the amino acid substitution may occur in a region of the protein that interacts with a taste receptor. In some cases, the substitution may enhance the interaction between curculin and the taste receptor. In some cases, the substitution may decrease the interaction between curculin and the taste receptor. The wild-type amino acid residue can be substituted with any suitable amino acid to enhance or increase the taste-modifying activity of the protein. In some cases, the substituted amino acid may have similar biochemical properties (e.g., charge, hydrophobicity, size) as the wild-type amino acid (e.g., conservative substitution). In some cases, the substituted amino acid may have different biochemical properties (e.g., charge, hydrophobicity, size) compared to the wild-type amino acid (e.g., non-conservative substitution).

A taste-modifying polypeptide comprising curculin (e.g., wild-type, fragment thereof, or mutant thereof) may bind to a sweet taste receptor with an appropriate dissociation constant.

### II. Methods of Producing Taste-Modifying Polypeptides

Taste-modifying agents of the present disclosure can be isolated from naturally occurring sources and produced recombinantly by a variety of protein expression systems, including, but not limited, to cell-based expression systems and cell-free expression systems. Non-limiting examples of protein expression systems useful in producing taste-modifying polypeptides disclosed herein include prokaryotic cell-based expression systems (e.g., archaeal systems, e.g., bacterial systems) and eukaryotic cell-based expression systems (e.g., fungal cells (filamentous fungal cells), e.g., yeast cells, insect cells, and mammalian cells).

To express a taste-modifying polypeptide disclosed herein in a cell-based expression system, a gene encoding for the polypeptide can be introduced into a host cell of the expression system by, for example, an expression vector. A polynucleotide gene encoding for the taste-modifying polypeptide can be made by various methods, including molecular cloning and synthesis. Molecular cloning methods may involve mutagenesis (e.g., site-directed mutagenesis), restriction enzyme-mediated cloning (e.g., restriction enzyme digestion and ligation), polymerase chain reaction (PCR), and overlap extension. Synthesis can include chemical synthesis (e.g., gene synthesis). The gene sequence can be codon optimized for any desired expression system.

In an aspect, the present disclosure provides a vector comprising a nucleic acid sequence encoding a taste-modifying polypeptide disclosed herein. In some cases, the present disclosure provides a vector comprising a nucleic acid sequence encoding for a taste-modifying polypeptide selected from the group consisting of; a brazzein polypeptide, a thaumatin polypeptide, a monellin polypeptide, a mabinlin polypeptide, a pentadin polypeptide, a miraculin polypeptide, or a curculin polypeptide. In some cases, the nucleic acid sequence may have at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to the sequence of wild-type brazzein shown in Table 1, thaumatin polypeptides shown in Table 2, monellin polypeptides shown in Table 3, mabinlin polypeptides shown in Table 4, miraculin shown in Table 5, or curculin shown in Table 6. The resulting polynucleotide gene can be inserted into a cloning vector and/or an expression vector.

The coding sequence can be inserted into a vector by a variety of procedures, including, but not limited to restriction enzyme digestion, ligation, and homologous recombination. The vector may be capable of replicating and expressing the polynucleotides in prokaryotic and/or eukaryotic host cells of expression systems. A vector may contain various components that may be adjusted and optimized for compatibility with the particular host cell. A cloning vector and/or expression vector may include additional nucleic acid sequences, including but not limited to, a signal sequence, an origin of replication, a marker gene (e.g., a selection marker such as an antibiotic resistance gene), an enhancer element, a promoter, a ribosome binding site, a signal sequence, and a transcription termination sequence.

In some aspects, an expression vector containing a polynucleotide encoding a taste-modifying polypeptide disclosed herein may further comprise a promoter. Promoters include, but are not limited to, constitutive promoters, inducible promoters, and hybrid promoters. Promoters include, but are not limited to, acu-5, adhl+, alcohol dehydrogenase (ADH1, ADH2, ADH4), AHSB4m, AINV, alcA, a-amylase, alternative oxidase (AOD), alcohol oxidase I (AOX1), alcohol oxidase 2 (AOX2), AXDH, B2, CaMV, cellobiohydrolase I (cbhl), ccg-1, cDNA1, cellular filament polypeptide (cfp), cpc-2, ctr4+, CUP1, dihydroxyacetone synthase (DAS), enolase (ENO, ENO1), formaldehyde dehydrogenase (FLD1), FMD, formate dehydrogenase (FMDH), Gl, G6, GAA, GAL1, GAL2, GAL3, GAL4, GAL5, GALE, GALT, GAL5, GAL5, GAL10, GCW14, gdhA, gla-1, a-glucoamylase (glaA), glyceraldehyde-3-phosphate dehydrogenase (gpdA, GAP, GAPDH), phosphoglycerate mutase (GPM1), glycerol kinase (GUT1), HSP82, invl+, isocitrate lyase (ICL1), acetohydroxy acid isomeroreductase (ILV5), KAR2, KEX2, β-galactosidase (lac4), LEU2, melO, MET3, methanol oxidase (MOX), nmtl, NSP, pcbC, PETS, peroxin 8 (PEX8), phosphoglycerate kinase (PGK, PGK1), phol, PH05, PH089, phosphatidylinositol synthase (PIS1), PYK1, pyruvate kinase (pkil), RPS7, sorbitol dehydrogenase (SDH), 3-phospho serine aminotransferase (SERI), SSA4, SV40, TEF, translation elongation factor 1 alpha (TEF1), THI11, homoserine kinase (THR1), tpi, TPS1, triose phosphate isomerase (TPI1), XRP2, and YPT1.

In some aspects, an expression vector containing a gene encoding a taste-modifying polypeptide disclosed herein may comprise an auxotrophic marker (e.g., adel, arg4, his4, ura3, met2).

In some aspects, an expression vector containing a gene encoding a taste-modifying polypeptide disclosed herein may comprise a selectable marker (e.g., a resistance gene). In some cases, a resistance gene may confer resistance to zeocin, ampicillin, blasticidin, kanamycin, nurseothricin, chloroamphenicol, tetracycline, triclosan, or ganciclovir.

In some instances, an expression vector containing a gene encoding a taste-modifying polypeptide disclosed herein may comprise a polynucleotide sequence encoding a signal peptide. A signal peptide, also known as a signal sequence, targeting signal, localization signal, localization sequence, secretion signal, transit peptide, leader sequence, or leader peptide, may support secretion of a protein or polynucleotide. Extracellular secretion of a recombinantly expressed protein from a host cell may facilitate protein purification. For example, recovery of a recombinant protein from a cell culture supernatant may be preferable to lysing host cells to release a complex mixture of proteins, including intracellular proteins of the host cell. Secretion, in some cases, may reduce deleterious effects that intracellular overexpression of a heterologous protein may have on a host cell such as toxicity or decreased growth rate. Secretion, in some cases, may allow increased protein production compared to intracellular expression in a host cell of limited volume to store the synthesized proteins. Secretory production of a protein, in some cases, may facilitate posttranslational modification or processing (e.g., protein folding, formation of disulfide bonds, and glycosylation).

The cloning and/or expression vector can then be introduced into a suitable host cell for replication/amplification and/or protein expression. Polynucleotides may be inserted into host cells by any means. Cells may be transformed by introducing an exogenous polynucleotide, for example, by direct uptake, endocytosis, transfection, F-mating, PEG-mediated protoplast fusion, Agrobacterium tumefaciens-mediated transformation, biolistic transformation, chemical transformation, or electroporation.

Once introduced, the exogenous polynucleotide can be maintained within the cell as a non-integrated expression vector (such as a plasmid) or integrated into the host cell genome. Once the vectors are introduced into host cells for protein production, host cells can be cultured in conventional nutrient media modified as appropriate for inducing promoters, selecting transformants, and/or amplifying the genes encoding the desired sequences.

As different host cells may have characteristics and specific mechanisms for the posttranslational processing and modification of protein products, appropriate cell lines or host systems may be chosen to ensure the desired modification and processing of the expressed protein. In some cases, a host cell may be selected from the group consisting of: bacteria, fungi, plant cells, insect cells, mammalian cells, and any combination thereof.

In some cases, a host cell selected for expression a taste-modifying polypeptide disclosed herein is an archaeal cell. Non-limiting examples of archaeal host cells include Pyrococcus furiosus, Metallosphera sedula, Thermococcus litoralis, Methanobacterium thermoautotrophicum, Methanococcus jannaschii, Pyrococcus abyssi, Sulfolobus solfataricus, Pyrococcus woesei, Sulfolobus shibatae, and variants thereof. In some cases, a host cell selected for expression a taste-modifying polypeptide disclosed herein is a bacterial cell. Suitable bacterial host cells include, but are not limited to, BL21 E. coli, DE3 strain E. coli, E. coli M15, DH5u, DH103, HB101, B. subtilis cells, Pseudomonas fluorescens cells, and cyanobacterial cells such as Synechococcus elongates cells.

Non-limiting expression vectors for use in archaeal and bacterial host cells include pCWori, pGEX vectors (e.g., pGEX-2T, pGEX-3X, pGEX-4T, pGEX-5X, pGEX-6P), pET vectors (e.g., pET-21, pET-21 a, pET-21 b, pET-22 pET-23, pET-24), pACYC vectors (e.g., pACYDuet-1), pDEST vectors (e.g., pDEST14, pDEST15, pDEST24, pDEST42), pBR322 and its derivatives, pQE vectors, pBluescript vectors, pNH vectors, lambda-ZAP vectors, ptrc99a, pKK223-3, pDR540, pRIT2T, pRSET, pCR-TOPO vectors, pSyn_vectors, pChlamy_1 vectors, pGEM1, and pMAL.

In some aspects, a host cell selected for expression of a taste-modifying polypeptide disclosed herein may be a fungal cell. The fungal cell may be a yeast cell or a filamentous fungi. Yeast may include, but is not limited to, Arxula spp., Arxula adeninivorans, Kluyveromyces spp., Kluyveromyces lactis, Pichia spp., Pichia angusta, Pichia pastoris, Saccharomyces spp., Saccharomyces cerevisiae, Schizosaccharomyces spp., Schizosaccharomyces pombe, Tetrahymena sp., Hansenula sp., Blastobotrys sp., Candida sp., Zygosaccharomyces sp., Debaryomyces sp. Yarrowia spp., and Yarrowia hpolytica. Fungi may include, but are not limited to, Agaricus spp., Agaricus bisporus, Aspergillus spp., Aspergillus awamori, Aspergillus fumigatus, Aspergillus nidulans, Aspergillus niger, Aspergillus oryzae, Colletotrichum spp., Colletotrichum gloeosporiodes, Endothia spp., Endothia parasitica, Fusarium spp., Fusarium graminearum, Fusarium solani, Mucor spp., Mucor miehei, Mucor pusillus, Myceliophthora spp., Myceliophthora thermophila, Neurospora spp., Neurospora crassa, Penicillium spp., Penicillium camemberti, Penicillium canescens, Penicillium chrysogenum, Penicillium (Talaromyces) emersonii, Penicillium funiculo sum, Penicillium purpurogenum, Penicillium roqueforti, Pleurotus spp., Pleurotus ostreatus, Rhizomucor spp., Rhizomucor miehei, Rhizomucor pusillus, Rhizopus spp., Rhizopus arrhizus, Rhizopus oligosporus, Rhizopus oryzae, Trichoderma spp., Trichoderma altroviride, Trichoderma reesei, and Trichoderma vireus.

In some aspects, a host cell selected for expression of a taste-modifying polypeptide disclosed herein may be an insect cell. Suitable insect host cells include, but are not limited to, Sf9 cells from Spodoptera frugiperda, Sf21 cells from Spodoptera frugiperda, Hi-Five cells, BTI-TN-5B1-4 Trichophusia ni cells, and Schneider 2 (S2) cells and Schneider 3 (S3) cells from Drosophila melanogaster.

In some aspects, a host cell selected for expression of a taste-modifying polypeptide disclosed herein may be a mammalian cell. Non-limiting examples of mammalian host cells include HEK293 cells, HeLa cells, CHO cells, COS cells, Jurkat cells, NS0 hybridoma cells, baby hamster kidney (BHK) cells, MDCK cells, NIH-3T3 fibroblast cells, and any other immortalized cell line derived from a mammalian cell.

Non-limiting examples of expression vectors for use in eukaryotic host cells include pXT1, pSG5, pSVK3, pBPV, pMSG, pSVLSV40, pcDNA3.3, pcDNA4/TO, pcDNA6/TR, pLenti6/TR, pMT vectors, pKLAC1 vectors, pKLAC2 vectors, pQE vectors, pYepSec1, pMFa, pJRY88, pYES2, PGAPZ, pTEF-MF, BacPak baculoviral vectors, pAdeno-X adenoviral vectors, and pBABE retroviral vectors.

Host cells used to produce the taste-modifying polypeptides disclosed herein can be grown in media suitable for culturing of the selected host cells. Non-limiting examples of suitable media for bacterial host cells include Luria broth (LB) plus necessary supplements, such as a selection agent, e.g., ampicillin. Non-limiting examples of suitable media for yeast host cells include YNB (yeast nitrogen base) media, YCB (yeast carbon base) media, YPD (yeast extract peptone dextrose) media, YPG (yeast extract peptone glycerol) media, and YPAc (yeast extract peptone acetate) media. Non-limiting examples of suitable media for mammalian host cells include Minimal Essential Medium (MEM), Dulbecco's Modified Eagle's Medium (DMEM), DMEM with supplemented fetal bovine serum (FBS), and RPMI-1640.

Host cells can be cultured at suitable temperature for protein expression and/or proper protein folding. In some instances, the host cells may be cultured at a temperature of about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., or about 39° C. In some instances, the host cells may be cultured at a temperature of between about 15° C. to about 39° C., e.g., between about 25° C. to about 37° C., between about 27° C. to about 35° C., or between about 29° C. to about 33° C.

The pH of the medium can be any suitable pH for protein expression and/or proper protein folding. In some instances, the pH of the medium may vary depending on the stage of protein expression (for example, host cell doubling with minimal protein production vs low host cell growth with maximum protein production). In some instances, the pH of the cell culture medium may be about pH 3.0, about pH 3.5, about pH 4.0, about pH 4.5, about pH 5.0, about pH 5.5, about pH 6.0, about pH 6.5, about pH 7.0, or about pH 7.5. In some instances, the pH of the cell culture medium may be between about pH 4.0 to about 8.0, e.g., between about pH 5.0 to about 7.0, or between about pH 5.5 to about 6.5. The pH may depend on the host organism.

A host cell may further be glycoengineered, for instance, by having its glycosylation pathways modified or engineered to more closely resemble another organism (e.g., a plant).

In an aspect, the present disclosure provides a recombinant yeast cell containing at least one copy of a stably integrated heterologous nucleic acid sequence encoding a taste-modifying protein, or a functional fragment thereof. In some instances, the taste-modifying protein may taste sweeter than a carbohydrate (e.g., a saccharide) and/or an artificial sweetener. In some instances, the taste-modifying protein may be itself flavorless. In some cases, the taste-modifying protein may enhance a taste, for example, a sweet taste. In some cases, the taste-modifying protein may have nearly zero calories for a predetermined serving size. In some cases, the taste-modifying protein may have a glycemic load of zero or approximately zero for a predetermined serving size. The taste-modifying protein may enhance a sweet taste in any suitable subject, such as a human, primate mammal, or non-primate mammal. In some cases, the taste-modifying protein may inhibit one or more bitter flavors.

In some aspects, the taste-modifying protein may bind to one or more taste receptors on a tongue of the subject. In some cases, the one or more taste receptors may comprise at least one type 1 taste receptor (TAS1R). The at least one type 1 taste receptor can be TAS1R1, TAS1R2, TAS1R3, or a combination thereof. In some cases, the one or more taste receptors may comprise at least one type 2 taste receptor (TAS2R). The at least one type 2 taste receptor can be TAS2R1, TAS2R50, TAS2R60, or a combination thereof.

The heterologous taste modifying nucleic acid sequence can encode for a miraculin protein or a functional fragment thereof, a brazzein protein or a functional fragment thereof, a curculin protein or a functional fragment thereof, a monellin protein or a functional fragment thereof, a thaumatin protein or a functional fragment thereof, a mabinlin protein or a functional fragment thereof, or a pentadin protein or a functional fragment thereof, in a yeast cell.

In some instances, the taste-modifying nucleic acid sequence may encode for a miraculin protein or a functional fragment thereof. In some cases, the recombinant yeast cell may secrete miraculin protein or a functional fragment thereof into a culture media. In some cases, the recombinant yeast cell may be of the phylum Ascomycota. In some cases, the recombinant yeast cell can be of the genus Komagataella or Kluyveromyces. In some cases, the recombinant yeast cell can be of the species Pichia pastoris. In some cases, the recombinant yeast cell can be of the species Kluyveromyces lactis.

The heterologous taste-modifying nucleic acid sequence can be codon optimized for expression in the yeast cell. The at least one heterologous nucleic acid sequence can be operably linked to an inducible promoter. The inducible promoter can comprise a nucleic acid sequence from an Aox1 promoter, an Aox2 promoter, or a Lac4 promoter. The at least one heterologous nucleic acid sequence can be operably linked to a constitutive promoter. The constitutive promoter can comprise a nucleic acid sequence from a GAP promoter.

In some aspects, taste-modifying polypeptides can be produced in in vitro translation systems. An in vitro translation system generally refers to a translation system which is a cell-free extract containing elements for translation of an RNA molecule into a protein. An in vitro translation system can comprise ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are available. Non-limiting examples of in vitro translation systems include eukaryotic lysates, such as rabbit reticulocyte lysates, rabbit oocyte lysates, human cell lysates, insect cell lysates, and wheat germ extracts.

Taste-modifying polypeptides produced using recombinant protein expression systems described herein may have comparable sweetness properties as proteins found in natural sources, such as plants. A difference in perceived sweetness between polypeptides produced recombinantly and polypeptides isolated from natural sources may vary by less than about 20% (e.g., less than 19%, 18%, 17%, 16%, 15%, 14%, 13%, 12%, 11%, 10%, 9%, 8%, 7%, 6%, 5%, 4%, 3%, 2%, 1%, or less). Taste-modifying polypeptides produced using recombinant protein expression systems described herein may have comparable taste-modifying properties as proteins found in natural sources. A difference in the extent of taste modification (e.g., perceived sweetness) between polypeptides produced recombinantly and polypeptides isolated from natural sources may vary by less than about 20% (e.g., less than 19%, 18%, 17%, 16%, 15%, 14%, 13%, 12%, 11%, 10%, 9%, 8%, 7%, 6%, 5%, 4%, 3%, 2%, 1%, or less). A difference in the length of time of taste modification between polypeptides produced recombinantly and polypeptides isolated from natural sources may vary by less than about 20% (e.g., less than 19%, 18%, 17%, 16%, 15%, 14%, 13%, 12%, 11%, 10%, 9%, 8%, 7%, 6%, 5%, 4%, 3%, 2%, 1%, or less).

The polypeptides produced recombinantly may be glycosylated. The glycosylation pattern of recombinantly produced polypeptides may be indistinguishable from the naturally occurring protein. In some cases, the glycosylation pattern of recombinantly produced polypeptides may be distinguishable from the naturally occurring protein.

In some aspects, a recombinantly produced polypeptide of the disclosure does not comprise xylose. In some cases, less than about 10% of the sugar moieties attached to a recombinantly produced polypeptide of the disclosure (e.g., by glycosylation) comprise xylose. For example, less than about 10%, less than about 9.5%, less than about 9%, less than about 8.5%, less than about 8%, less than about 7.5%, less than about 7%, less than about 6.5%, less than about 6%, less than about 5.5%, less than about 5%, less than about 4.5%, less than about 4%, less than about 3.5%, less than about 3%, less than about 2.5%, less than about 2%, less than about 1.5%, less than about 1%, or less than about 0.5% of the sugar moieties attached to a recombinantly produced polypeptide of the disclosure (e.g., by glycosylation) comprise xylose.

A difference in molecular weight, for example, between polypeptides produced recombinantly and polypeptides isolated from natural sources may vary by less than about 20% (e.g., less than 19%, 18%, 17%, 16%, 15%, 14%, 13%, 12%, 11%, 10%, 9%, 8%, 7%, 6%, 5%, 4%, 3%, 2%, 1%, or less). In some cases, the recombinantly expressed protein may have a higher molecular weight than a protein isolated from natural sources. In some cases, the recombinantly expressed protein may have a lower molecular weight than a protein isolated from natural sources. A difference in protein stability, for further example, between polypeptides produced recombinantly and polypeptides isolated from natural sources may vary by less than about 20% (e.g., less than 19%, 18%, 17%, 16%, 15%, 14%, 13%, 12%, 11%, 10%, 9%, 8%, 7%, 6%, 5%, 4%, 3%, 2%, 1%, or less). In some cases, the recombinantly expressed protein may be more stable than proteins isolated from natural sources. In some cases, the recombinantly expressed protein may be less stable than proteins isolated from natural sources.

For taste-modifying polypeptides comprising multiple polypeptide chains, the recombinantly expressed protein may be non-covalently associated. In some cases, the polypeptide chains may be linked by disulfide bonds. In some cases, the recombinantly expressed proteins may occur as monomers, dimers (e.g., heterodimers, homodimers), or tetramers.

### III. Recovery and Purification of Taste-Modifying Polypeptides

Expressed taste-modifying polypeptides described herein can be recovered and/or purified from the supernatant of a cell culture, for example, if the expressed protein is secreted by the host cell into the cell culture medium. Expressed taste-modifying polypeptides described herein can, in some cases, be recovered from the host cell, for example, if the protein is expressed intracellularly. Proteins can be recovered from the intracellular space by disrupting the host cell, for example, by osmotic shock, sonication, or lysis. Once the cells are disrupted, cell debris may be removed by centrifugation or filtration, and the expressed proteins can be removed or recovered from the cell lysate.

In some aspects, the polypeptide may be produced using in vitro or cell-free protein synthesis systems, for example, using a cell-free translation system comprising a cell extract such as Escherichia coli cell extract, rabbit reticulocyte cell extract, wheat germ cell extract, or insect cell extract. The expressed protein may be recovered, isolated, and/or optionally purified from the cell extract by any suitable method.

Polypeptides can be recovered and/or isolated from supernatants and lysates by any of a variety of methods, including, but not limited to, chemical extraction, column chromatography, and filtration. The polypeptides may be purified using any of a variety of methods including liquid chromatography such as normal or reversed phase, high-performance liquid chromatography (HPLC), fast protein liquid chromatography (FPLC), and the like; affinity chromatography such as with inorganic ligands, monoclonal antibodies (e.g., immunoaffinity), and ion exchange (e.g., anion exchange, cation exchange); hydrophobic interaction chromatography; size exclusion chromatography; immobilized metal chelate chromatography; gel electrophoresis; ethanol precipitation; and any combination thereof. In some cases, the polypeptides can be purified by centrifugation and/or filtration, including sterile filtration, depth filtration, tangential flow filtration, ultrafiltration (UF), diafiltration (DF), and ultrafiltration/diafiltration (UF/DF).

In an aspect, the present disclosure provides a process for isolating a heterologous taste-modifying protein, or a functional fragment thereof, from a recombinant yeast cell culture media. In some cases, the recombinant yeast can be of the phylum Ascomycota. In some cases, the recombinant yeast can be of the genus Komagataella or Kluyveromyces. In some cases, the recombinant yeast can be of the species Pichia pastoris. In some cases, the recombinant yeast can be of the species Kluyveromyces lactis.

In some aspects, the process may comprise filtering a supernatant having the taste-modifying protein, or functional fragment thereof, from the yeast cell culture media through a porous membrane, thereby isolating the protein. In some cases, the process may comprise incubating the recombinant yeast cell for a period of time so that the yeast cell secretes the taste-modifying protein into the supernatant.

The taste-modifying protein can be any polypeptide provided herein. The taste-modifying protein can be selected from the group consisting of: a miraculin protein or functional fragment thereof, a brazzein protein or functional fragment thereof, a curculin protein or a functional fragment thereof, a monellin protein or a functional fragment thereof, a thaumatin protein or a functional fragment thereof, a mabinlin protein or a functional fragment thereof, and a pentadin protein or a functional fragment thereof. In some cases, the heterologous protein may be a taste-modifying protein, and the taste-modifying protein may be miraculin or a functional fragment thereof. In some cases, the heterologous protein may be a taste-modifying protein, and the taste-modifying protein may be brazzein or a functional fragment thereof.

The porous membrane can have pores of any suitable size. The pore size can be selected to either exclude the heterologously expressed polypeptide and retain undesired contaminants or exclude undesired contaminants and retain the heterologously expressed polypeptide. In some cases, the porous membrane may comprise pores ranging from about 0.01 μm to about 0.5 μm in diameter, from about 0.1 μm to about 0.4 μm in diameter, or from about 0.2 μm to about 0.3 μm in diameter. The porous membrane can comprise pores that are at least about 0.01 μm, about 0.02 μm, about 0.03 μm, about 0.04 μm, about 0.05 μm, about 0.06 μm, about 0.07 μm, about 0.08 μm, about 0.09 μm, about 0.1 μm, about 0.2 μm, about 0.3 μm, about 0.4 μm, about 0.5 μm, about 0.6 μm, about 0.7 μm, about 0.8 μm, about 0.9 μm, or about 1 μm in diameter.

In some aspects, the process may comprise adjusting the pH of the filtered supernatant. For example, the filtered supernatant can be adjusted to an acidic pH. In some cases, the filtered supernatant may be adjusted to a basic pH.

In some aspects, contaminants may remain in the recovered polypeptide sample. In some cases, one or more contaminants present in the recovered protein may be from a host cell. For example, the mixture may comprise contaminants selected from host cell proteins, host cell metabolites, host cell constitutive proteins, nucleic acids, endotoxins, viruses, product related contaminants, lipids, media additives and media derivatives. In a specific instance, the cell culture is a yeast cell culture, such as a K. lactis cell culture, and the one or more contaminants comprise yeast cell proteins, yeast cell metabolites, and/or yeast cell nucleic acids.

In some aspects, the total contaminant content may be less than about 20% weight/weight (w/w), less than about 19% w/w, less than about 18% w/w, less than about 17% w/w, less than about 16% w/w, less than about 15% w/w, less than about 14% w/w, less than about 13% w/w, less than about 12% w/w, less than about 11% w/w, less than about 10% w/w, less than about 9% w/w, less than about 8% w/w, less than about 7% w/w, less than about 6% w/w, less than about 5% w/w, less than about 4% w/w, less than about 3% w/w, less than about 2% w/w, less than about 1% w/w, less than about 0.9% w/w, less than about 0.8% w/w, less than about 0.7% w/w, less than about 0.6% w/w, less than about 0.5% w/w, less than 0.4% about w/w, less than about 0.3% w/w, less than about 0.2% w/w, less than about 0.1% w/w, or less.

In some cases, the contaminant concentration may be about 1 to about 10 parts per million (ppm), about 1 to about 20 ppm, about 1 to about 30 ppm, about 1 to about 40 ppm, about 1 to about 50 ppm, about 1 to about 100 ppm, about 1 to about 1,000 ppm, about 10 to about 100 ppm, about 20 to about 100 ppm, about 50 to about 200 ppm, or about 50 to about 500 ppm. In some cases, the contaminant concentration is about 1 ppm, about 5 ppm, about 10 ppm, about 15 ppm, about 20 ppm, about 25 ppm, about 30 ppm, about 35 ppm, about 40 ppm, or about 50 ppm. In some cases, the contaminant concentration is less than about 1000 ppm, about 500 ppm, about 400 ppm, about 300 ppm, about 200 ppm, about 150 ppm, about 100 ppm, about 75 ppm, about 50 ppm, about 40 ppm, about 30 ppm, about 20 ppm, or about 10 ppm.

The purified polypeptides can be stored in any suitable buffer or medium and at any desired concentration. In some cases, the polypeptide may be stored in a liquid carrier selected from the group consisting of: water, an alcohol, propylene glycol, triacetine, medium chain triglycerides, glycerin, and combinations thereof. In some cases, the liquid carrier may be water.

In some cases, the polypeptide may be stored in solid form. In some cases, the polypeptide may be stored in crystalline form. In some cases, the polypeptide may be in amorphous form. In some cases, the polypeptide may be coating a solid carrier. In some cases, the solid carrier may be particles selected from the group consisting of: lactose, modified food starch, gum Arabic, maltodextrin, modified corn starch, dextrose, xantham gum, carboxymethylcellulose, cellulose gel, cellulose gum, sodium caseinate, carrageenan, and combinations thereof.

In some aspects, the total polypeptide content is at least about 70% weight/weight (w/w), at least about 71% w/w, at least about 72% w/w, at least about 73% w/w, at least about 74% w/w, at least about 75% w/w, at least about 76% w/w, at least about 77% w/w, at least about 78% w/w, at least about 79% w/w, at least about 80% w/w, at least about 81% w/w, at least about 82% w/w, at least about 83% w/w, at least about 84% w/w, at least about 85% w/w, at least about 86% w/w, at least about 87% w/w, at least about 88% w/w, at least about 89% w/w, at least about 90% w/w, at least about 91% w/w, at least about 92% w/w, at least about 93% w/w, at least about 94% w/w, at least about 95% w/w, at least about 96% w/w, at least about 97% w/w, at least about 98% w/w, or at least about 99% w/w.

In some aspects, the total polypeptide content is at least about 0.01 mg/mL, at least about 0.02 mg/mL, at least about 0.03 mg/mL, at least about 0.04 mg/mL, at least about 0.05 mg/mL, at least about 0.06 mg/mL, at least about 0.07 mg/mL, at least about 0.08 mg/mL, at least about 0.09 mg/mL, at least about 0.1 mg/mL, at least about 0.2 mg/mL, at least about 0.3 mg/mL, at least about 0.4 mg/mL, at least about 0.5 mg/mL, at least about 0.6 mg/mL, at least about 0.7 mg/mL, at least about 0.8 mg/mL, at least about 0.9 mg/mL, or at least about 1.0 mg/mL.

IV. Recovery of Taste-Modifying Polypeptides from Plants

In some aspects, taste-modifying agents of the present disclosure can be isolated from naturally occurring sources. Taste-modifying agents of the present disclosure can also be produced recombinantly by a variety of protein expression systems, including, but not limited, to cell-based expression systems and cell-free expression systems, as previously described.

In an aspect, the present disclosure provides methods for purifying taste-modifying polypeptides from plants and/or plant extracts. Plants or berries thereof containing the taste-modifying polypeptides can be homogenized to yield plant extracts. Plant cells can be subjected to plant cell lysis to yield plant extracts. Taste-modifying polypeptides in extracts and lysates can be recovered and/or isolated by any of a variety of methods, including, but not limited to, chemical extraction, column chromatography, and filtration. The polypeptides may be purified using any of a variety of methods including liquid chromatography such as normal or reversed phase, high-performance liquid chromatography (HPLC), fast protein liquid chromatography (FPLC) and the like; affinity chromatography such as with inorganic ligands, monoclonal antibodies (e.g., immunoaffinity), and ion exchange (e.g., anion exchange, cation exchange); hydrophobic interaction chromatography; size exclusion chromatography; immobilized metal chelate chromatography; gel electrophoresis; ethanol precipitation; and any combination thereof. The polypeptides can be purified by centrifugation and/or filtration, including sterile filtration, depth filtration, tangential flow filtration, ultrafiltration (UF), diafiltration (DF), and ultrafiltration/diafiltration (UF/DF).

In some aspects, a process for purifying a protein from a plant extract can comprise first filtering the plant extract having the taste-modifying polypeptide, or functional fragment or mutant thereof, through a porous membrane to obtain a first filtrate. The porous membrane can have pores of any suitable size. The pore size can be selected to either exclude the polypeptide and retain undesired contaminants or exclude undesired contaminants and retain the polypeptide. In some cases, the porous membrane may comprise pores ranging from about 0.01 μm to about 0.5 μm in diameter, from about 0.1 μm to about 0.4 μm in diameter, or from about 0.2 μm to about 0.3 μm in diameter. The porous membrane can comprise pores that are at least about 0.01 μm, about 0.02 μm, about 0.03 μm, about 0.04 μm, about 0.05 μm, about 0.06 μm, about 0.07 μm, about 0.08 μm, about 0.09 μm, about 0.1 μm, about 0.2 μm, about 0.3 μm, about 0.4 μm, about 0.5 μm, about 0.6 μm, about 0.7 μm, about 0.8 μm, about 0.9 μm, or about 1 μm in diameter.

In some cases, a hydrostatic pressure may be applied to force an amount of a liquid against a semipermeable membrane. In some cases, the liquid forced against the semipermeable membrane may flow at a rate ranging from about 550 ml/minute to about 650 ml/minute, e.g., from about 575 ml/minute to about 625 ml/minute. In some cases, the flow rate of the liquid may be about 550 ml/minute, about 575 ml/minute, about 600 ml/minute, about 625 ml/minute, or about 650 ml/minute.

In some cases, the hydrostatic pressure can range from about 20 psi to about 30 psi, about 22 psi to about 28 psi, or about 24 psi to about 36 psi. In some cases, the hydrostatic pressure may be about 15 psi, about 20 psi, about 25 psi, about 30 psi, or about 35 psi. The plant extract can be centrifuged prior to filtering. Next, the first filtrate can be filtered through a nickel affinity column, thereby obtaining a purified protein from the plant extract. In some cases, the protein may comprise a histidine tag comprising at least two histidine amino acid residues.

The purified protein can be dialyzed from the plant extract. The process can comprise adjusting the pH of the filtered supernatant. For example, the filtered supernatant can be adjusted to an acidic pH. In some cases, the filtered supernatant may be adjusted to a basic pH.

Contaminants may remain in the recovered polypeptide sample. One or more contaminants present in the recovered protein may be from the plant and/or plant cells. For example, the mixture may comprise contaminants selected from the group consisting of: plant cell proteins, plant cell metabolites, plant cell constitutive proteins, nucleic acids, endotoxins, viruses, and any combination thereof.

In some aspects, the total contaminant content may be less than about 20% weight/weight (w/w), less than about 19% w/w, less than about 18% w/w, less than about 17% w/w, less than about 16% w/w, less than about 15% w/w, less than about 14% w/w, less than about 13% w/w, less than about 12% w/w, less than about 11% w/w, less than about 10% w/w, less than about 9% w/w, less than about 8% w/w, less than about 7% w/w, less than about 6% w/w, less than about 5% w/w, less than about 4% w/w, less than about 3% w/w, less than about 2% w/w, less than about 1% w/w, less than about 0.9% w/w, less than about 0.8% w/w, less than about 0.7% w/w, less than about 0.6% w/w, less than about 0.5% w/w, less than about 0.4% w/w, less than about 0.3% w/w, less than about 0.2% w/w, less than about 0.1% w/w, or less.

In some aspects, the contaminant concentration may be about 1 to about 10 parts per million (ppm), about 1 to about 20 ppm, about 1 to about 30 ppm, about 1 to about 40 ppm, about 1 to about 50 ppm, about 1 to about 100 ppm, about 1 to about 1,000 ppm, about 10 to about 100 ppm, about 20 to about 100 ppm, about 50 to about 200 ppm, or about 50 to about 500 ppm. In some aspects, the contaminant concentration may be about 1 ppm, about 5 ppm, about 10 ppm, about 15 ppm, about 20 ppm, about 25 ppm, about 30 ppm, about 35 ppm, about 40 ppm, or about 50 ppm. In some aspects, the contaminant concentration may be less than about 1000 ppm, about 500 ppm, about 400 ppm, about 300 ppm, about 200 ppm, about 150 ppm, about 100 ppm, about 75 ppm, about 50 ppm, about 40 ppm, about 30 ppm, about 20 ppm, or about 10 ppm.

### V. Compositions Comprising Taste-Modifying Polypeptides

The present disclosure further provides compositions comprising taste-modifying polypeptides which can be administered to a subject. The composition can be administered orally, for example, as an oral dosage. Non-limiting examples of taste-modifying polypeptides contemplated in compositions herein include any taste-modifying polypeptides (or functional fragments thereof) described herein, including the proteins brazzein, pentadin, thaumatin (e.g., thaumatin I, thaumatin II), monellin, mabinlin (e.g., mabinlin-1, mabinlin-2, mabinlin-3, mabinlin-4), miraculin, curculin. In some instances, the taste-modifying polypeptide may be recombinantly expressed (e.g., in a recombinant yeast, recombinant bacteria, or recombinant mammalian cell) and purified according to the disclosure. In some instances, the taste-modifying polypeptide may be naturally produced (e.g., by a natural plant source). In a non-limiting example, the taste-modifying polypeptide may be unpurified from a natural plant source (e.g., freeze dried berry powder containing therein the taste-modifying polypeptide). In another non-limiting example, the taste-modifying polypeptide may be purified from a natural plant source. Compositions of the disclosure can take the form of liquids, aerosols, and solids, non-limiting examples of which include solutions, elixirs, syrups, beverages (e.g., coffee, tea, juices, sodas), slurries, suspensions, emulsions, colloids, aerosols, vapors, sprays, mists, films, tablets, pills, capsules, gels, jellies, compotes, purees, gelatins, lozenges, hard candies, ice cream, frozen treats (e.g., popsicles) and other frozen preparations, wafers, powders, sustained-release formulations, coatings, and the like.

The compositions can be formulated in a unit dosage form. The term “unit dosage form(s),” as used herein, refers to physically discrete units suitable as unitary dosages for human subjects and other mammals, each unit containing a predetermined quantity of taste-modifying polypeptide calculated to produce the desired gustatory effect. The amount of purified polypeptide or functional fragment thereof per unit dose may be selected to achieve a desired level and/or duration of taste-modifying polypeptide activity, for example, in a human subject, and may further depend on the particular formulation. The term “dose,” as used herein, refers to the quantity of taste-modifying polypeptide administered to a subject all at one time (unit dose), or in two or more administrations over a defined time interval.

In an aspect, the present disclosure provides an oral dosage form comprising a taste-modifying polypeptide. In some aspects, the taste-modifying polypeptide can be a brazzein protein or any functional fragment or mutant thereof. In some cases, the taste-modifying polypeptide can be a recombinantly expressed brazzein protein or any functional fragment or mutant thereof. In some cases, the recombinantly expressed brazzein protein or any functional fragment or mutant thereof may be purified. In some cases, the brazzein protein may be naturally produced (e.g., by a natural plant source). The naturally produced brazzein protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of wild-type brazzein in Table 1.

In some aspects, the taste-modifying polypeptide can be a pentadin protein or any functional fragment or mutant thereof. In some cases, the taste-modifying polypeptide can be a recombinantly expressed pentadin protein or any functional fragment or mutant thereof. In some cases, the recombinantly expressed pentadin protein or any functional fragment or mutant thereof may be purified. In some cases, the pentadin protein may be naturally produced (e.g., by a natural plant source). The naturally produced pentadin protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of wild-type pentadin.

In some aspects, the taste-modifying polypeptide can be a thaumatin protein (e.g., thaumatin I, thaumatin II) or any functional fragment or mutant thereof. In some cases, the taste-modifying polypeptide can be a recombinantly expressed thaumatin protein (e.g., thaumatin I, thaumatin II), or any functional fragment or mutant thereof. In some cases, the recombinantly expressed thaumatin protein or any functional fragment or mutant thereof may be purified. In some cases, the thaumatin protein may be naturally produced (e.g., by a natural plant source). The naturally produced thaumatin protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of wild-type thaumatin I or thaumatin II in Table 2.

In some aspects, the purified taste-modifying polypeptide can be a monellin protein or any functional fragment or mutant thereof. In some cases, the taste-modifying polypeptide can be a recombinantly expressed monellin protein or any functional fragment or mutant thereof. In some cases, the recombinantly expressed monellin protein or any functional fragment or mutant thereof may be purified. In some cases, the monellin protein may be naturally produced (e.g., by a natural plant source). The naturally produced monellin protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of wild-type monellin in Table 3.

In some aspects, the purified taste-modifying polypeptide can be a mabinlin protein (e.g., mabinlin-1, mabinlin-2, mabinlin-3, mabinlin-4) or any functional fragment or mutant thereof. In some cases, the taste-modifying polypeptide can be a recombinantly expressed mabinlin protein (e.g., mabinlin-1, mabinlin-2, mabinlin-3, mabinlin-4), or any functional fragment or mutant thereof. In some cases, the recombinantly expressed mabinlin protein or any functional fragment or mutant thereof may be purified. In some cases, the mabinlin protein may be naturally produced (e.g., by a natural plant source). The naturally produced mabinlin protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of a wild-type mabinlin protein (e.g., mabinlin-1, mabinlin-2, mabinlin-3, mabinlin-4) in Table 4.

In some aspects, the purified taste-modifying polypeptide can be a miraculin protein or any functional fragment or mutant thereof. In some cases, the taste-modifying polypeptide can be a recombinantly expressed miraculin protein or any functional fragment or mutant thereof. In some cases, the recombinantly expressed miraculin protein or any functional fragment or mutant thereof may be purified. In some cases, the miraculin protein may be naturally produced (e.g., by a natural plant source). The naturally produced miraculin protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of wild-type miraculin in Table 5.

In some aspects, the purified taste-modifying polypeptide can be a curculin protein or any functional fragment or mutant thereof. In some cases, the taste-modifying polypeptide can be a recombinantly expressed curculin protein or any functional fragment or mutant thereof. In some cases, the recombinantly expressed curculin protein or any functional fragment or mutant thereof may be purified. In some cases, the curculin protein may be naturally produced (e.g., by a natural plant source). The naturally produced curculin protein may be unpurified or purified from the natural plant source. The taste-modifying polypeptide can be at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence of wild-type curculin in Table 6.

In some aspects, the oral dosage form may comprise at least about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, about 0.500 mg, about 0.525 mg, about 0.550 mg, about 0.575 mg, about 0.600 mg, about 0.625 mg, about 0.650 mg, about 0.675 mg, about 0.700 mg, about 0.725 mg, about 0.750 mg, about 0.775 mg, about 0.800 mg, about 0.825 mg, about 0.850 mg, about 0.875 mg, about 0.900 mg, about 0.925 mg, about 0.950 mg, about 0.975 mg, or about 1000 mg of purified polypeptide per unit dose.

In some aspects, the oral dosage form may comprise at most about 0.500 mg, about 0.475 mg, about 0.450 mg, about 0.425 mg, about 0.400 mg, about 0.375 mg, about 0.350 mg, about 0.325 mg, about 0.300 mg, about 0.275 mg, about 0.250 mg, about 0.225 mg, about 0.200 mg, about 0.175 mg, about 0.150 mg, about 0.125 mg, about 0.100 mg, about 0.095 mg, about 0.090 mg, about 0.085 mg, about 0.080 mg, about 0.075 mg, about 0.070 mg, about 0.065 mg, about 0.060 mg, about 0.055 mg, about 0.050 mg, about 0.045 mg, about 0.040 mg, about 0.035 mg, about 0.030 mg, about 0.025 mg, about 0.020 mg, about 0.015 mg, about 0.010 mg, about 0.009 mg, about 0.008 mg, about 0.007 mg, about 0.006 mg, about 0.005 mg, about 0.004 mg, about 0.003 mg, about 0.002 mg, or about 0.001 mg of purified polypeptide per unit dose.

In some cases, the oral dosage form can comprise from about 0.001 mg to about 0.5 mg, about 0.001 mg to about 0.4 mg, about 0.001 mg to about 0.3 mg, about 0.001 mg to about 0.2 mg, about 0.001 mg to about 0.1 mg, about 0.001 mg to about 0.090 mg, about 0.001 mg to about 0.080 mg, about 0.001 mg to about 0.070 mg, about 0.001 mg to about 0.060 mg, about 0.001 mg to about 0.050 mg, about 0.001 mg to about 0.040 mg, about 0.001 mg to about 0.030 mg, about 0.001 mg to about 0.020 mg, about 0.001 mg to about 0.010 mg, about 0.001 mg to about 0.009 mg, about 0.001 mg to about 0.008 mg, about 0.001 mg to about 0.007 mg, about 0.001 mg to about 0.006 mg, about 0.001 mg to about 0.005 mg, about 0.001 mg to about 0.004 mg, about 0.001 mg to about 0.003 mg, or about 0.001 mg to about 0.002 mg of purified polypeptide per unit dose.

In some aspects, the total polypeptide content in an oral dosage form may be about 1% weight/weight (w/w), about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, about 30% w/w, about 31% w/w, about 32% w/w, about 33% w/w, about 34% w/w, about 35% w/w, about 36% w/w, about 37% w/w, about 38% w/w, about 39% w/w, about 40% w/w, about 41% w/w, about 42% w/w, about 43% w/w, about 44% w/w, about 45% w/w, about 46% w/w, about 47% w/w, about 48% w/w, about 49% w/w, about 50% w/w, about 51% w/w, about 52% w/w, about 53% w/w, about 54% w/w, about 55% w/w, about 56% w/w, about 57% w/w, about 58% w/w, about 59% w/w, about 60% w/w, about 61% w/w, about 62% w/w, about 63% w/w, about 64% w/w, about 65% w/w, about 66% w/w, about 67% w/w, about 68% w/w, about 69% w/w, about 70% w/w, about 71% w/w, about 72% w/w, about 73% w/w, about 74% w/w, about 75% w/w, about 76% w/w, about 77% w/w, about 78% w/w, about 79% w/w, about 80% w/w, about 81% w/w, about 82% w/w, about 83% w/w, about 84% w/w, about 85% w/w, about 86% w/w, about 87% w/w, about 88% w/w, about 89% w/w, about 90% w/w, about 91% w/w, about 92% w/w, about 93% w/w, about 94% w/w, about 95% w/w, about 96% w/w, about 97% w/w, about 98% w/w, or about 99% w/w.

Taste-modifying polypeptides disclosed herein can be added to or mixed with one or more additives. Additives can be used, for example, to facilitate the processes associated with the preparation of compositions, e.g., dosage forms, described herein. These processes may include, for example, agglomeration, air suspension chilling, air suspension drying, balling, coacervation, comminution, compression, pelletization, cryopelletization, encapsulation, extrusion, granulation, homogenization, inclusion complexation, lyophilization, nanoencapsulation, melting, mixing, molding, pan coating, dehydration, solvent dehydration, sonication, spheronization, spray chilling, spray congealing, spray drying, or other processes.

Suitable additives utilized in various embodiments described herein may include, by way of non-limiting example, adsorbing agents, anti-adherents, anticoagulants, antifoaming agents, antioxidants, anti-caking agents, anti-static agents, binders, bile acids, bufferants, bulking agents (also referred to as excipients), chelating agents, coagulants, colorants, co-solvent, opaquants, congealing agents, coolants, cryoprotectants, diluents, dehumidifying agents, desiccants, desensitizers, disintegrants, dispersing agents, enzyme inhibitors, glidants, fillers, hydrating agent, super disintegrants, gums, mucilages, hydrogen bonding agents, enzymes, flavorants, humectants, humidifying agents, lubricant oils, ion-exchange resins, lubricants, plasticizers, pH modifying agents, preservatives, solidifying agent, solvents, solubilizers, spreading agents, sweeteners, stabilizers, surface area enhancing agents, suspending agents, thickeners, viscosity increasing agents, waxes and mixtures thereof. In some cases, an additive can add volume and/or mass to a composition, e.g., an oral dosage form. In some cases, an additive may improve functional performance and/or physical characteristics of the composition. In some cases, an additive may increase the shelf-life of the composition. Additives are preferably non-toxic to recipients at the dosages and concentrations employed.

A composition herein can comprise a food coloring. Non-limiting examples of food colorings that can be mixed with taste-modifying polypeptides in compositions herein include FD&C Yellow #5, FD&C Yellow #6, FD&C Red #40, FD&C Red #3, FD&C Blue No. 1, FD&C Blue No. 2, FD&C Green No. 3, carotenoids (e.g., saffron, β-carotene), annatto, betanin, butterfly pea, caramel coloring, chlorophyllin, elderberry juice, lycopene, carmine, pandan, paprika, turmeric, curcuminoids, quinoline yellow, carmoisine, Ponceau 4R, Patent Blue V, and Green S.

A composition herein can comprise a pH adjuster, also referred to as a buffer. Non-limiting examples of pH adjusters that can be mixed with taste-modifying polypeptides in compositions herein include Tris buffer, potassium phosphate, sodium hydroxide, potassium hydroxide, citric acid, sodium citrate, sodium bicarbonate, and hydrochloric acid. A pH adjuster may act as a pH stabilizer.

A composition herein can comprise a stabilizer or a binding agent. Non-limiting examples of stabilizers and binding agents that can be mixed with taste-modifying polypeptides in compositions herein include kappa carrageenan, iota carrageenan, lambda carrageenan, triethyl citrate, xanthan gum, methyl cellulose, hydroxyethyl cellulose, hydroxymethyl cellulose, and polyacrylamides.

A composition herein can comprise an emulsifying agent. Non-limiting examples of emulsifiers that can be mixed with taste-modifying polypeptides in compositions herein include a surfactant, a polysaccharide, a lectin, and a phospholipid.

In some cases, the emulsifier may be a surfactant. Non-limiting examples of surfactants include polysorbate, for example, polysorbate 20 (TWEEN® 20), polysorbate 40 (TWEEN® 40), polysorbate 60 (TWEEN® 60), polysorbate 61 (TWEEN® 61), polysorbate 65 (TWEEN® 65), polysorbate 80 (TWEEN® 80), and polysorbate 81 (TWEEN® 81); poloxamer (polyethylene-polypropylene copolymers), for example, Poloxamer 124 (PLURONIC® L44), Poloxamer 181 (PLURONIC® L61), Poloxamer 182 (PLURONIC® L62), Poloxamer 184 (PLURONIC® L64), Poloxamer 188 (PLURONIC® F68), Poloxamer 237 (PLURONIC® F87), Poloxamer 338 (PLURONIC® L108), Poloxamer 407 (PLURONIC® F127); polyoxyethyleneglycol dodecyl ether, for example, BRIJ® 30, and BRIJ® 35; 2-dodecoxyethanol (LUBROL®-PX); polyoxyethylene octyl phenyl ether (TRITON® X-100); sodium dodecyl sulfate (SDS); 34(3-cholamidopropyl)dimethylammoniol-1-propanesulfonate (CHAPS); 3-[(3-cholamidopropyl)dimethylammonio]-2-hydroxy-1-propanesulfonate (CHAPSO); sucrose monolaurate; and sodium cholate.

In some cases, the emulsifier may be a polysaccharide. Non-limiting examples of polysaccharides include guar gum, agar, alginate, calgene, a dextran (e.g., dextran 1K, dextran 4K, dextran 40K, dextran 60K, and dextran 70K), dextrin, glycogen, inulin, starch (e.g., cornstarch, tapioca starch), a starch derivative (such as hydroxymethyl starch, hydroxyethyl starch, hydroxypropyl starch, hydroxybutyl starch, and hydroxypentyl starch), hetastarch, cellulose, FICOLL, methyl cellulose (MC), carboxymethyl cellulose (CMC), hydroxyethyl cellulose (HEC), hydroxypropyl cellulose (HPC), hydroxyethyl methyl cellulose (NEMC), hydroxypropyl methyl cellulose (HPMC); polyvinyl acetates (PVA); polyvinyl pyrrolidones (PVP), also known as povidones, having a K-value of less than or equal to 18, a K-value greater than 18 or less than or equal to 95, or a K-value greater than 95, such as PVP 12 (KOLLIDON® 12), PVP 17 (KOLLIDON® 17), PVP 25 (KOLLIDON® 25), PVP 30 (KOLLIDON® 30), PVP 90 (KOLLIDON® 90); and polyethylene imines (PEI).

In some cases, the emulsifier may be a lectin. Non-limiting examples of lectins that can be used in compositions comprising taste-modifying polypeptides herein include mannose-binding lectins, galactose/N-acetylgalactosamine-binding lectins, N-acetylgluxosamine-binding lectins, N-acetylneuramine-binding lectins, N-acetylneuraminic acid-binding lectins, and fucose-binding lectins. Non-limiting examples of lectins include concanavain A, lentil lectin, snowdrop lectin, Roin, peanut agglutinin, j acain, hairy vetch lectin, wheat germ agglutinin, elderberry lectin, Maackia anurensis leukoagglutinin, Maackia anurensis hemoagglutinin, Ulex europaeus agglutinin, and Aleuria aurantia lectin.

In some cases, the emulsifier may be a phospholipid. Non-limiting examples of phospholipids that can be used in compositions comprising taste-modifying polypeptides herein include diacylglycerides and phosphosphingolipids. Non-limiting examples of diacylglycerides include a phosphatidic acid (phosphatidate) (PA), a phosphatidylethanolamine (cephalin) (PE), a phosphatidylcholine (lecithin) (PC), a phosphatidylserine (PS), and a phosphoinositide including phosphatidylinositol (PI), phosphatidylinositol phosphate (PIP), phosphatidylinositol bisphosphate (PIP2), and phosphatidylinositol triphosphate (PIPS). Non-limiting examples of phosphosphingolipids include a ceramide phosphorylcholine (sphingomyelin) (SPH), ceramide phosphorylethanolamine (sphingomyelin) (Cer-PE), and ceramide phosphorylglycerol.

A composition herein can comprise a salt. Non-limiting examples of salts that can be mixed with taste-modifying polypeptides in compositions herein include acid salt, alkali salt, organic salt, inorganic salt, phosphates, chloride salts, sodium chloride, potassium chloride, magnesium chloride, magnesium perchlorate, calcium chloride, ammonium chloride, iron chlorides, and zinc chloride.

Compositions of the disclosure comprising taste-modifying polypeptides may comprise a humectant. Humectants, which can also be referred to as wetting agents, can be incorporated into compositions herein to promote the retention of moisture. In some cases, humectants can control the amount of water that enters or exits the composition. A humectant may be a hydrous or an anhydrous solvent. Non-limiting examples of humectants that can be mixed with taste-modifying polypeptides in compositions herein include mineral oil, glycerin, glycerol formal, miglyol (e.g., miglyol 812, miglyol 840), Solutol HS 15 (polyglycol mono- and di-esters of 12-hydroxystearic acid), ethylene glycol, propylene glycol, methoxypropanol, diethylene glycol monoethyl ether, diethylene glycol monomethyl ether, diethylene glycol monobutyl ether, tetraglycol, triethylene glycol, butyl diglycol, dimethylacetamide, dimethylformamide, n-methylformamide, dipropylene glycol n-butyl ether, ethanol, isopropanol, methanol, ethylene glycol monoethyl ether, ethylene glycol monomethyl ether, dipropyleneglycol monomethyl ether, dipropyleneglycol monomethyl ether, dipropyleneglycol monomethyl ether, triethylene glycol monoethyl ether, triethylene glycol monomethyl ether, polyethylene glycols, methoxypolyethylene glycols, polypropylene glycols, polybutylene glycols, diethylene monoethylether acetate, diethylene monobutylether acetate, monomethylacetamide, liquid polyoxyethylene glycols, 2-pyrrolidone, propylene carbonate, butylene carbonate, tetrahydrofurfuryl alcohol, solketal, xylene, dimethyl isosorbide, short-, medium- and long chain, and aromatic fatty acids, for example butyric acid, capric acid, succinic, adipic, sebacic, capriylic acid, lauric acid, myristic acid, strearic acid, linoleic acid, and benzoic acid, triglycerides, for example, castor oil, cottonseed oil, sesame oil, linseed oil, safflower oil, peanut oil, soybean oil, coconut oil, olive oil, corn oil, and almond oil. A composition herein can comprise a humectant such as glycerin, glycerol, sorbitol, polyethylene glycol, propylene glycol, and an edible polyhydric alcohol. In some cases, compositions herein may comprise glycerin. Glycerin in compositions herein may be vegetable glycerin made from vegetable oils (e.g., palm oil, palm stearin, palm kernel oil, coconut oil, and soybean oil), animal glycerin from animal fats, or synthetic glycerin produced from cane syrup sugar, corn syrup sugar, or propylene.

The humectant may be present in the composition at any suitable concentration, for example, to maintain a desired level of water in the composition. In some cases, a composition comprising a taste-modifying polypeptide may comprise a humectant at a concentration of at least about 0.01% w/w, about 0.05% w/w, about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1.0% w/w, about 1.5% w/w, about 2.0% w/w, about 2.5% w/w, about 3.0% w/w, about 3.5% w/w, about 4.0% w/w, about 4.5% w/w, or about 5.0% w/w. In some cases, a composition comprising a taste-modifying polypeptide may comprise a humectant at a concentration of no more than about 5.0% w/w, about 4.5% w/w, about 4.0% w/w, about 3.5% w/w, about 3.0% w/w, about 2.5% w/w, about 2.0% w/w, about 1.5% w/w, about 1.0% w/w, about 0.9% w/w, about 0.8% w/w, about 0.7% w/w, about 0.6% w/w, about 0.5% w/w, about 0.4% w/w, about 0.3% w/w, about 0.2% w/w, about 0.1% w/w, about 0.05% w/w, or about 0.01% w/w.

A composition herein can comprise a nutrient. Non-limiting examples of nutrients that can be mixed with taste-modifying polypeptides in compositions herein include macronutrients, micronutrients, essential nutrients, non-essential nutrients, dietary fiber, amino acids, essential fatty acids, omega-3 fatty acids, and conjugated linoleic acid.

A composition herein can comprise a carbohydrate. Non-limiting examples of carbohydrates that can be mixed with taste-modifying polypeptides in compositions herein include sugar, sucrose, glucose, fructose, galactose, lactose, maltose, mannose, allulose, tagatose, xylose, arabinose, high fructose corn syrup, high maltose corn syrup, corn syrup (e.g., glucose-free corn syrup), monosaccharides, disaccharides, and polysaccharides (e.g., polydextrose, maltodextrin).

A composition herein can comprise a gum. Non-limiting examples of gums that can be mixed with taste-modifying polypeptides in compositions herein include gum arabic, gellan gum, guar gum, locust bean gum, acacia gum, cellulose gum, and xanthan gum.

Preservatives and antioxidants can be mixed with polypeptides in compositions herein to prevent or delay the deterioration of the formulation. Non-limiting examples of preservatives that can be mixed with taste-modifying polypeptides in compositions herein include methyl paraben, propyl paraben, quaternary ammonium salts, benzalkonium chloride, esters of p-hydroxybenzoic acid, and boric acid. Non-limiting examples of antioxidants that can be mixed with taste-modifying polypeptides herein include free radical scavengers such as tocopherols, alkyl gallates, butylated hydroxyanisole, and butylated hydroxytoluene; reducing agents such as ascorbic acid and sodium metabisulfite; and antioxidant synergists such as citric acid, tartaric acid, and lecithin.

A composition herein can comprise a vitamin. Non-limiting examples of vitamins that can be mixed with taste-modifying polypeptides in compositions herein include niacin, riboflavin, pantothenic acid, thiamine, folic acid, vitamin A, vitamin B6, vitamin B12, vitamin D, vitamin E, lutein, zeaxanthin, choline, inositol, and biotin.

A composition herein can comprise a dietary element. Non-limiting examples of dietary elements that can be mixed with taste-modifying polypeptides in compositions herein include calcium, iron, magnesium, phosphorus, potassium, sodium, zinc, copper, manganese, selenium, chlorine, iodine, sulfur, cobalt, molybdenum, and bromine.

Additives disclosed herein, while described as having particular properties, may have a combination of properties and be useful for a variety of purposes. For example, citric acid may be used in compositions herein as a preservative as well as a flavoring agent.

### VI. Preparations of Taste-Modifying Polypeptides

For oral administration, compositions comprising taste-modifying polypeptides can take the form of powders, liquids, beverages (e.g., coffee, tea, juices, sodas), solutions, elixirs, syrups, slurries, suspensions, emulsions, colloids, aerosols, vapors, sprays, mists, films, tablets, pills, capsules, gels, jellies, compotes, gelatins, lozenges, hard candy, wafers, sustained-release formulations, coatings, and the like.

Compositions comprising taste-modifying polypeptides can be formulated to have any suitable dissolution profile when administered as an oral dose. A dissolution rate of said oral dosage form can be more than about 80% (e.g., more than about 81%, 82%, 83%, 84%, 85%, 86%, 87%, 88%, 89%, 90%, 91%, 92%, 93%, 94%, 95%, 96%, 97%, 98%, or 99%) within about 5 minutes, 10 minutes, 15 minutes, 20 minutes 25 minutes, or 30 minutes following entry of the dosage form into a use environment (e.g., oral cavity). The oral dosage form may not exhibit dose-proportionality.

In some aspects, compositions of the disclosure may have less calories per serving as compared to an equivalent composition comprising sugar. For example, composition of the disclosure may have 50% less calories, 55% less calories, 60% less calories, 65% less calories, 70% less calories, 75% less calories, 80% less calories, 85% less calories, 90% less calories, or 95% less calories than an equivalent composition comprising sugar.

In some aspects, a composition comprising a taste-modifying polypeptide may comprise less sugar than an equivalent composition not containing a taste-modifying polypeptide. For example, a composition comprising a taste-modifying polypeptide may comprise 50% less sugar, 55% less sugar, 60% less sugar, 65% less sugar, 70% less sugar, 75% less sugar, 80% less sugar, 85% less sugar, 90% less sugar, or 95% less sugar than an equivalent composition not containing a taste-modifying polypeptide.

### A. Aerosol

Compositions comprising taste-modifying polypeptides can be administered orally in the form of an aerosol. In some aspects, an aerosol may comprise a purified taste-modifying polypeptide. In some cases, the purified taste-modifying polypeptide may comprise a recombinantly expressed taste-modifying polypeptide, as provided herein. In some cases, the purified taste-modifying polypeptide may comprise a taste-modifying polypeptide purified from a natural source (e.g., a plant) as disclosed herein. In some aspects, an aerosol may comprise an unpurified taste-modifying polypeptide from a natural source (e.g., whole berry powder). In some cases, an aerosol of the disclosure may comprise any taste-modifying polypeptide as disclosed herein, either recombinantly produced or naturally produced. For example, an aerosol of the disclosure may comprise one or more of: brazzein, pentadin, thaumatin, monellin, mabinlin, miraculin, and curculin; or any functional fragment thereof; or any mutant thereof.

An aerosol comprising taste-modifying polypeptides can be administered, for example, by inhalation. A composition of the present disclosure can be suspended or dissolved in an appropriate carrier, e.g., a non-toxic propellant, and administered directly into the oral cavity. For example, an aerosol formulation comprising a taste-modifying polypeptide can be dissolved, suspended or emulsified in a propellant or a mixture of solvent and propellant, e.g., for administration as an oral spray or inhalant. Aerosol formulations can contain any acceptable propellant under pressure, such as a cosmetically or dermatologically or pharmaceutically acceptable propellant.

An aerosol formulation for inhalations and inhalants can be designed so that the taste-modifying polypeptide is applied to the oral cavity, for example, of a human subject. Inhalation solutions can be administered, for example, by a nebulizer. Inhalations comprising finely powdered or liquid compositions can be delivered to the oral cavity as an aerosol of a solution or suspension of the taste-modifying polypeptide in a propellant, e.g., to aid in disbursement. Propellants can be liquefied gases, including halocarbons, for example, fluorocarbons such as fluorinated chlorinated hydrocarbons, hydrochlorofluorocarbons, and hydrochlorocarbons, as well as hydrocarbons and hydrocarbon ethers.

Halocarbon propellants can include fluorocarbon propellants in which all hydrogens are replaced with fluorine, chlorofluorocarbon propellants in which all hydrogens are replaced with chlorine and at least one fluorine, hydrogen-containing fluorocarbon propellants, and hydrogen-containing chlorofluorocarbon propellants. Hydrocarbon can include, for example, propane, isobutane, n-butane, pentane, isopentane and neopentane. A blend of hydrocarbons can also be used as a propellant. Ether propellants include, for example, dimethyl ether as well as the ethers. An aerosol formulation of the disclosure can also comprise more than one propellant. For example, the aerosol formulation can comprise more than one propellant from the same class, such as two or more fluorocarbons; or more than one, more than two, more than three propellants from different classes, such as a fluorohydrocarbon and a hydrocarbon. Compositions of the present disclosure can also be dispensed with a compressed gas, e.g., an inert gas such as carbon dioxide, nitrous oxide or nitrogen.

Aerosol formulations can also include other components, for example, ethanol, isopropanol, propylene glycol, as well as surfactants or other components such as oils and detergents. These components can serve to stabilize the formulation and/or lubricate valve components.

The aerosol formulation can be packaged under pressure and can be formulated as an aerosol using solutions, suspensions, emulsions, powders and semisolid preparations. For example, a solution aerosol formulation can comprise a solution of a taste-modifying polypeptide in (substantially) pure propellant or as a mixture of propellant and solvent. The solvent can be used to dissolve the polypeptide and/or retard the evaporation of the propellant. Solvents may include, for example, water, ethanol and glycols. Any combination of suitable solvents can be used and optionally combined with preservatives, antioxidants, and/or other aerosol components. Antimicrobial agents, antifungal agents, and/or preservatives can also be included in the formulation.

An aerosol formulation for oral administration can be an aqueous solution designed to be administered to the oral cavity in drops or sprays. An aerosol formulation can also be a dispersion or suspension. A suspension aerosol formulation can comprise a suspension of a taste-modifying polypeptide and a dispersing agent. Dispersing agents useful in the present disclosure include, for example, sorbitan trioleate, oleyl alcohol, oleic acid, lecithin and corn oil. A suspension aerosol formulation can also include lubricants, preservatives, antioxidant, and/or other aerosol components.

An aerosol formulation comprising taste-modifying polypeptides can comprise any suitable amount of taste-modifying polypeptide. The aerosol formulation can comprise, per dose or single administration, about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, about 0.500 mg, about 0.525 mg, about 0.550 mg, about 0.575 mg, about 0.600 mg, about 0.625 mg, about 0.650 mg, about 0.675 mg, about 0.700 mg, about 0.725 mg, about 0.750 mg, about 0.775 mg, about 0.800 mg, about 0.825 mg, about 0.850 mg, about 0.875 mg, about 0.900 mg, about 0.925 mg, about 0.950 mg, about 0.975 mg, or about 1000 mg of purified taste-modifying polypeptide. An aerosol formulation comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide per does or per single administration.

### B. Liquids

In some aspects, a composition of the disclosure may include a liquid (e.g., an aqueous suspension) comprising a purified taste-modifying polypeptide. In some cases, the purified taste-modifying polypeptide may comprise a recombinantly expressed taste-modifying polypeptide, as provided herein. In some cases, the purified taste-modifying polypeptide may comprise a taste-modifying polypeptide purified from a natural source (e.g., plant source) as disclosed herein. In some aspects, a liquid composition of the disclosure may comprise an unpurified taste-modifying polypeptide from a natural source (e.g., whole berry powder). In some cases, a liquid composition of the disclosure may comprise any taste-modifying polypeptide as disclosed herein, either recombinantly produced or naturally produced. For example, a liquid composition of the disclosure may comprise one or more of: brazzein, pentadin, thaumatin, monellin, mabinlin, miraculin, and curculin; or any functional fragment thereof or any mutant thereof.

Liquid compositions (e.g., an aqueous suspension) comprising taste-modifying polypeptides can be administered orally. The suspension can be ingested as a liquid or applied to the oral cavity as drops or a mist. The suspension can be, for example, a solution, an elixir, a syrup, a slurry, a suspension, or an emulsion. Aqueous suspensions for oral use can contain a taste-modifying polypeptide with an aqueous solvent and one or more acceptable excipients, such as a suspending agent (e.g., methyl cellulose), a wetting agent (e.g., glycerin, lecithin, lysolecithin and/or a long-chain fatty alcohol), as well as coloring agents, preservatives, flavoring agents, and the like.

A liquid composition comprising taste-modifying polypeptides can comprise any suitable amount of taste-modifying polypeptide. A liquid composition can comprise, per dose, about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, about 0.500 mg, about 0.525 mg, about 0.550 mg, about 0.575 mg, about 0.600 mg, about 0.625 mg, about 0.650 mg, about 0.675 mg, about 0.700 mg, about 0.725 mg, about 0.750 mg, about 0.775 mg, about 0.800 mg, about 0.825 mg, about 0.850 mg, about 0.875 mg, about 0.900 mg, about 0.925 mg, about 0.950 mg, about 0.975 mg, or about 1000 mg of purified taste-modifying polypeptide. A liquid composition comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide.

In the case of a liquid, a dose may refer to any suitable volume of liquid to deliver the desired amount of taste-modifying polypeptide. For example, a dose may refer to a volume of about 1 ml, about 2 ml, about 3 ml, about 4 ml, about 5 ml, about 6 ml, about 7 ml, about 8 ml, about 9 ml or about 10 ml of a solution of taste-modifying polypeptide at a particular concentration of taste-modifying polypeptide. In the case of a spray, a dose may refer to any number of sprays to deliver a desired amount of taste-modifying polypeptide. For example, the total number of sprays in a dose may add up to a total volume of about 1 ml, about 2 ml, about 3 ml, about 4 ml, or about 5 ml of a solution of taste-modifying polypeptide at a particular concentration of taste modifying polypeptide. In some cases, a dose comprises at least 1, 2, 3, 4, or 5 sprays.

In some formulations, oils or non-aqueous solvents may be used. Alternatively, emulsions, suspensions, or other preparations, for example, liposomal preparations, can be used.

### C. Thin Films

In some aspects, a composition disclosed herein comprising a taste-modifying polypeptide may be formulated as a thin film. In some cases, a thin film composition of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a thin film composition of the disclosure may comprise an unpurified taste-modifying polypeptide from a natural source (e.g., whole berry powder). In some cases, a thin film composition of the disclosure may comprise any taste-modifying polypeptide as disclosed herein, either recombinantly produced or naturally produced. For example, a thin film composition of the disclosure may comprise one or more of: brazzein, pentadin, thaumatin, monellin, mabinlin, miraculin, and curculin; or any functional fragment thereof; or any mutant thereof.

The thin film can be administered orally. When placed in an oral cavity, a thin film can dissolve or disintegrate therein and release taste-modifying polypeptides from the film. In some cases, a thin film can be applied to an object, for example, a food utensil, and the taste-modifying polypeptide can be administered orally when a food is consumed with the object, e.g., food utensil. In an example, the thin film can be applied to a spoon and the thin film can be dissolved by food or saliva when the spoon is used to consume the food. In another example, the thin film can be applied to the inside of a straw. The thin film can be dissolved by a liquid drink when the straw is used to consume the liquid drink.

A thin film (which can also be referred to as an “oromucosal film preparation”, “orodispersible film”, or a “melt film”) can comprise a combination of at least one carrier (e.g., film former or film forming agent) and a taste-modifying polypeptide. Film formers can incorporate taste-modifying polypeptides and influence the structure, shape, and dissolution behavior of the film preparation. Non-limiting examples of film formers that can be used in thin films comprising taste-modifying polypeptides herein include cellulose, cellulose derivatives, acrylic and methacrylic acid polymers, polyvinyl alcohols, polyvinyl acetate, polyvinylpyrrolidone and derivatives, polysaccharides and polymers based on starch.

Non-limiting examples of film formers which can be used herein include hydroxyethylcellulose, hydroxypropylcellulose, hydroxypropylmethylcellulose, methylcellulose, Natrosol®, Tylose® H300, Klucel®, Klucel® E, Klucel® L, Klucel® J, Klucel® G, Klucel® M, Klucel® H, Klucel® EF, Klucel® LF, Klucel® JF, Klucel® GF, Klucel® MF, Klucel® HF, Klucel® EXF, Klucel® LXF, Klucel® JXF, Klucel® GXF, Klucel® MXF, Klucel® HXF, Pharmacoat® 603, Pharmacoat® 606, Methocel® E4M, polyethylene glycol-polyvinyl alcohol copolymers, Kollicoat® IR, Kollicoat® protect, sodium carboxymethylcellulose, polysaccharides of maltotriose, Pullulan®, Lycoat® RS720, Lycoat® RS780, Lycoat® NG, and hydroxyethylmethylcellulose.

Thin films may be prepared using other ingredients, including, but not limited to, a food acid, a salt of a food acid, a buffering system, a bulking agent, a sequestrant, a cross-linking agent, one or more flavors, one or more colors, and combinations thereof.

Non-limiting examples of food acids for use in particular embodiments include citric acid and ascorbic acid. In some aspects, a thin film of the present disclosure can comprise Lycoat® RS780, hydroxypropyl methylcellulose (HPMC), a food coloring, citric acid, ascorbic acid, glycerin (e.g., vegetable glycerin), water, and a taste-modifying polypeptide.

The combination of film former and taste-modifying polypeptide can be shaped to yield a thin film. The thin film, when placed in the oral cavity, can dissolve or disintegrate in the oral cavity under the influence of moisture and release the taste-modifying polypeptide. The dimensions of the thin film may be selected to achieve a desired dissolution rate and/or profile. The dimensions of the film can be any suitable dimensions to provide a desired amount of taste-modifying polypeptide, for example in the units of g/dose, mg/dose, or μg/dose.

In some aspects, the thin film is in the shape of a rectangle, having a length and a width. In some cases, the film is about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in length. In some cases, the film is at least about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in length. In some cases, the film is about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in width. In some cases, the film is at least about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in width. The thin film can be in the shape of a square. The thin film can be in the shape of a circle.

The thickness of the film can be about 50 μm, about 75 μm, about 100 μm, about 125 μm, about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, about 700 μm, about 725 μm, about 750 μm, about 775 μm, about 800 μm, about 825 μm, about 850 μm, about 875 μm, about 900 μm, about 925 μm, about 950 μm, about 975 μm, or about 1000 μm. The thickness of such a film can be less than about 1 mm, e.g., less than about 500 μm, less than about 400 μm, less than about 300 μm, less than about 200 μm, or less than about 100 μm.

A thin film comprising taste-modifying polypeptides can comprise any suitable amount of taste-modifying polypeptide. For example, each thin film can comprise about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, or about 0.500 mg of purified taste-modifying polypeptide. A thin film comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide.

Non-limiting examples of oral thin films comprising miraculin are disclosed in Example 7 and 22. In some cases, the thin film may comprise recombinant miraculin. In some cases, the thin film may comprise miraculin purified from a natural source (e.g., plant). In some cases, the amount of purified miraculin may be from about 0.1% w/w to about 1.0% w/w. For example, the amount of purified miraculin in a thin film formulation may be about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, or about 1.0% w/w. In some cases, the thin film may comprise miracle berry powder.

### D. Chewables

In some aspects, a composition comprising a taste-modifying polypeptide described herein can be formulated as a chewable. In some cases, a chewable composition comprises any taste-modifying polypeptide as disclosed herein. In some cases, the purified taste-modifying polypeptide is a recombinantly expressed taste-modifying polypeptide. In some cases, the purified taste-modifying polypeptide may comprise a taste-modifying polypeptide purified from a natural source (e.g., a plant source) as disclosed herein. In some aspects, a chewable composition of the disclosure may comprise an unpurified taste-modifying polypeptide from a natural source (e.g., whole berry powder). In some cases, a chewable composition of the disclosure may comprise any taste-modifying polypeptide as disclosed herein, either recombinantly produced or naturally produced. For example, a chewable composition of the disclosure may comprise one or more of: brazzein, pentadin, thaumatin, monellin, mabinlin, miraculin, and curculin; or any functional fragment thereof; or any mutant thereof.

The chewable can, in some cases, be in a solid form. Non-limiting examples of solid forms include a tablet, a pill, a powder, a capsule, solid dispersion, a solid solution, a bioerodible dosage form, controlled release formulations, pulsatile release dosage forms, multi-particulate dosage forms, pellets, or granules. Taste-modifying polypeptides disclosed herein can be administered as a single capsule or in multiple capsule dosage form.

In some aspects, a composition in solid form can be an edible gel or fruit leather which can be administered orally. Edible gels and fruit leathers can be dissolved in the oral cavity and/or masticated, thereby releasing the taste-modifying polypeptide from the gel or leather. A gel generally refers to a colloidal system in which a network of particles spans the volume of a liquid medium. Although gels may be primarily composed of liquids, and thus exhibit densities similar to liquids, gels can have the structural coherence of solids due to the network of particles that spans the liquid medium. Gels can generally appear to be solid, jelly-like materials

Non-limiting examples of edible gel compositions for use herein include gel desserts, puddings, jellies, pastes, trifles, aspics, marshmallows, gummy candies, and the like. Edible gel mixes may be powdered or granular solids, for example, powdered or solid gelling agents or ingredients, to which a fluid can be added to form an edible gel composition. Non-limiting examples of fluids for use in embodiments herein include water, dairy fluids, dairy analogue fluids, juices, alcohol, alcoholic beverages, coffee and coffee-like beverages, tea and tea-like beverages, and combinations thereof. Non-limiting examples of dairy fluids which may be used in particular embodiments include milk, cultured milk, cream, fluid whey, yogurt, and mixtures thereof. Non-limiting examples of dairy analogue fluids which may be used in particular embodiments include, for example, soy milk, nut milks, and non-dairy coffee whitener. Non-limiting examples of juices which may be used herein include, for example, grape juice, apple juice, orange juice, and cranberry juice. As used herein, the terms “gelling agent” and “gelling ingredient” refer to any material that can form a colloidal system within a liquid medium. Non-limiting examples of gelling ingredients include gelatin, alginate, carageenan, gum, pectin, konjac, agar, food acid, rennet, starch, starch derivatives, and combinations thereof. A variety of starches are available for use, non-limiting examples of which include starches from corn/maize, wheat, rice, buckwheat, barley, oat, millet, rye, sorghum, potato, sweet potato, tapioca/cassava, arrowroot, sago, arracacha, banana, plantain, malanga, kudzu, oca, canna, taro, yams, chestnuts, edible beans (e.g., lentils, favas, mung bean), and peas. In some cases, starch derivatives can be used. Starch derivatives can, in some cases, be modified starches subjected to processes including, but not limited to, hydroxypropylation and cross-linking. The amount of gelling ingredient used in an edible gel mix or an edible gel composition can vary depending on a number of factors, such as the gelling ingredient used, the fluid base used, and the desired properties of the gel.

Edible gel mixes and edible gels may be prepared using other ingredients, including, but not limited to, a food acid, a salt of a food acid, a buffering system, a bulking agent, a sequestrant, a cross-linking agent, one or more flavors, one or more colors, and combinations thereof. Non-limiting examples of food acids for use in particular aspects include citric acid and ascorbic acid.

In some aspects, an edible gel of the present disclosure can comprise gelatin as the gelling ingredient, cranberry juice as the fluid base, citric acid, ascorbic acid, water, and the taste-modifying polypeptide.

In some aspects, an edible gel of the present disclosure may be a gummy candy. In some cases, the gummy candy is as described in Example 11 or Example 20. In a particular aspect, a gummy candy of the disclosure may include one of more of the following ingredients: gelatin, water, juice (e.g., apple juice concentrate, cranberry juice), starch (e.g., corn starch), guar gum, citric acid, ascorbic acid, polysorbate 20, flavor oil (e.g., blackberry flavor oil), food color (e.g., red food color), and the taste-modifying polypeptide.

In some cases, a gummy candy may comprise a recombinant taste-modifying polypeptide. In some cases, a gummy candy may comprise a taste-modifying polypeptide purified from a natural source (e.g., a plant source). In some cases, a gummy candy may comprise a taste-modifying polypeptide unpurified from a natural source (e.g., berry powder). In some cases, a gummy candy not containing a taste-modifying polypeptide (e.g., a sour gummy candy) may be consumed with a formulation (e.g., another gummy candy) containing the taste-modifying polypeptide. In such cases, the sour gummy candy, when consumed with a formulation comprising a taste-modifying polypeptide, may taste sweet.

A gummy candy comprising taste-modifying polypeptides can comprise any suitable amount of taste-modifying polypeptide. For example, a gummy candy may comprise about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, or about 0.500 mg of purified taste-modifying polypeptide. A gummy candy comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide.

In some cases, the gummy candy may comprise recombinant miraculin. In some cases, the gummy candy may comprise miraculin purified from a natural source (e.g., plant). In some cases, the amount of purified miraculin may be from about 0.001% w/w to about 0.050% w/w. For example, the amount of purified miraculin in a gummy candy may be about 0.001% w/w, about 0.005% w/w, about 0.010% w/w, about 0.015% w/w, about 0.020% w/w, about 0.025% w/w, about 0.030% w/w, about 0.035% w/w, about 0.040% w/w, about 0.045% w/w, or about 0.050% w/w. In some cases, the gummy candy may comprise miracle berry powder. In some cases, the amount of miracle berry powder may be from about 0.1% w/w to about 15% w/w. For example, the amount of miracle berry powder in a yogurt formulation may be about 0.1% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, about 5.0% w/w, about 5.5% w/w, about 6.0% w/w, about 6.5% w/w, about 7.0% w/w, about 7.5% w/w, about 8.0% w/w, about 8.5% w/w, about 9.0% w/w, about 9.5% w/w, about 10.0% w/w, about 10.5% w/w, about 11.0% w/w, about 11.5% w/w, about 12.0% w/w, about 12.5% w/w, about 13.0% w/w, about 13.5% w/w, about 14.0% w/w, about 14.5% w/w, or about 15.0% w/w. In some cases, a gummy candy may comprise both purified miraculin and miracle berry powder.

An edible gel can be shaped to yield any shape. In some cases, an edible gel can be shaped to yield a block shape, for example, a cube. The gel, when placed in the oral cavity, can be dissolved or masticated in the oral cavity. The dimensions of the gel can be any suitable dimensions to provide a desired amount of taste-modifying polypeptide, for example, in the units of g/dose, mg/dose, or μg/dose. The gel can be in the shape of a block, having a length, a width, and a height. In some cases, the gel is about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in length. In some cases, the gel is at least about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in length. In some cases, the gel is about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in width. In some cases, the gel is at least about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in width. In some cases, the gel is about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in height. In some cases, the gel is at least about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in height. The gel can be in the shape of a cube. While a rectangular shape is described herein, any shape may be used. In some cases, the shape can be geometric. In some cases, the shape may not be geometric.

As used herein, the term “fruit leather” refers to an edible food product that has a texture similar to leather. Fruit leathers can be prepared from a puree of a fruit and dried to the consistency of leather. In an example, a fruit leather can be prepared by combining starch (e.g., tapioca starch), hydroxypropyl methylcellulose (HPMC), polysorbate 20, citric acid, ascorbic acid, glycerin (e.g., vegetable glycerin), water, and a taste-modifying polypeptide. The mixture can be dehydrated, for example, by forced-air flow.

A fruit leather can be shaped to any yield any desirable form. The fruit leather, when placed in the oral cavity, can be dissolved or masticated in the oral cavity. The dimensions of the fruit leather can be any suitable dimensions to provide a desired amount of taste-modifying polypeptide, for example, in the units of g/dose, mg/dose, or μg/dose. The fruit leather can be a thin layer. The thin layer may be, in some cases, rolled and/or folded upon itself to yield a three dimensional structure. In some cases, the fruit leather may be shaped into a cylinder, having a length and a diameter. In some cases, the fruit leather may be about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in diameter. In some cases, the fruit leather may be at least about 0.1 cm, about 0.2 cm, about 0.3 cm, about 0.4 cm, about 0.5 cm, about 0.6 cm, about 0.7 cm, about 0.8 cm, about 0.9 cm, about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in diameter. In some cases, the fruit leather may be about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in length. In some cases, the fruit leather may be at least about 1 cm, about 1.25 cm, about 1.5 cm, about 1.75 cm, about 2.0 cm, about 2.25 cm, about 2.50 cm, about 2.75 cm, about 3.0 cm, about 3.25 cm, about 3.5 cm, about 3.75 cm, about 4.0 cm, about 4.25 cm, about 4.5 cm, about 4.75 cm, or about 5.0 cm in length.

In some aspects, a composition comprising a taste-modifying polypeptide described herein can be formulated as a tablet. In some cases, a tablet composition of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a tablet composition of the disclosure may comprise an unpurified taste-modifying polypeptide from a natural source (e.g., whole berry powder). In some cases, a tablet composition of the disclosure may comprise any taste-modifying polypeptide as disclosed herein, either recombinantly produced or naturally produced. For example, a thin film composition of the disclosure may comprise one or more of: brazzein, pentadin, thaumatin, monellin, mabinlin, miraculin, and curculin; or any functional fragment thereof or any mutant thereof.

A tablet can be, without limitation, a suspension tablet, a fast-melt tablet, a bite-disintegration tablet, a rapid-disintegration tablet, an effervescent tablet, or a caplet. In some cases, taste-modifying polypeptides can be mixed with a carrier having the necessary binding capacity in suitable proportions and compacted in the shape and size desired. Suitable carriers include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Taste modifying polypeptides can be included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage.

In some aspects, a composition comprising a taste-modifying polypeptide described herein can be a powder. In some cases, a powder composition of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a powder composition of the disclosure may comprise an unpurified taste-modifying polypeptide from a natural source (e.g., whole berry powder). In some cases, a powder composition of the disclosure may comprise any taste-modifying polypeptide as disclosed herein, either recombinantly produced or naturally produced. For example, a powder composition of the disclosure may comprise one or more of: brazzein, pentadin, thaumatin, monellin, mabinlin, miraculin, and curculin; or any functional fragment thereof; or any mutant thereof.

Non-limiting examples of powders include, a sterile packaged powder, a dispensable powder, or an effervescent powder. A powder formulation can, in some cases, be a finely divided solid which is a mixture with finely divided taste modifying polypeptides. For example, a powder formulation can be made by finely dividing a tablet or a pill.

A composition comprising a taste-modifying polypeptide described herein can be a capsule. In some cases, a capsule composition of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a capsule composition of the disclosure may comprise an unpurified taste-modifying polypeptide from a natural source (e.g., whole berry powder). In some cases, a capsule composition of the disclosure may comprise any taste-modifying polypeptide as disclosed herein, either recombinantly produced or naturally produced. For example, a capsule composition of the disclosure may comprise one or more of: brazzein, pentadin, thaumatin, monellin, mabinlin, miraculin, and curculin; or any functional fragment thereof; or any mutant thereof.

Capsules can be soft capsules or hard capsules. Capsules can be made, for example, from animal-derived gelatin or plant-derived HPMC.

A chewable comprising taste-modifying polypeptides can comprise any suitable amount of taste-modifying polypeptide. In some cases, a chewable can comprise about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, or about 0.500 mg of purified taste-modifying polypeptide. A chewable comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide.

### E. Beverages

In some aspects, a composition disclosed herein comprising a taste-modifying polypeptide may be formulated as a liquid beverage. In some cases, a liquid beverage composition of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a liquid beverage composition of the disclosure may comprise an unpurified taste-modifying polypeptide from a natural source (e.g., whole berry powder). In some cases, a liquid beverage composition of the disclosure may comprise any taste-modifying polypeptide as disclosed herein, either recombinantly produced or naturally produced. For example, a liquid beverage composition of the disclosure may comprise one or more of: brazzein, pentadin, thaumatin, monellin, mabinlin, miraculin, and curculin; or any functional fragment thereof; or any mutant thereof.

In some cases, the liquid beverage may be coffee or a coffee product. In some cases, the liquid beverage may be tea or a tea product. In some cases, the liquid beverage may be soda or a soda product. In some cases, the liquid beverage may be juice (e.g., fruit juice) or a juice product. In some cases, the liquid beverage may be water. Other non-limiting examples of liquid beverages envisioned herein include, dairy milk and other liquid dairy products, nut “milks” (e.g., almond milk), other “milk” products (e.g., soy milk, coconut milk, rice milk), kombucha, alcohol and alcoholic beverages (e.g., beer, wine, spirits, cider), and hot chocolate.

In some cases, the taste-modifying polypeptide may be added directly to or mixed in to the liquid beverage formulation. In other cases, the taste-modifying polypeptide may be in a separate formulation that accompanies the liquid beverage product. In a non-limiting example, the liquid beverage may be a concentrated coffee product provided in a single-use disposable container or pod (e.g., K-Cups® for use with a Keurig® coffee maker). In another non-limiting example, the taste-modifying polypeptide may be loaded into a bottle cap (e.g., a soda bottle cap) to be consumed with the liquid beverage. In some cases, the taste-modifying polypeptide may be provided as a film or coating of a straw (e.g., on the inner surface of the hollow portion of the straw, or on the outer surface of the mouthpiece portion of the straw), which may be supplied separately or with a liquid beverage.

A liquid beverage comprising taste-modifying polypeptides can comprise any suitable amount of taste-modifying polypeptide. For example, a liquid beverage may comprise about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, or about 0.500 mg of purified taste-modifying polypeptide. A liquid beverage comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide.

### F. Yogurt Products

In some aspects, a composition disclosed herein comprising a taste-modifying polypeptide may be formulated as a yogurt product. In some cases, a yogurt product of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a yogurt product of the disclosure may comprise an unpurified taste-modifying polypeptide from a natural source (e.g., berry powder). In some cases, a yogurt product of the disclosure may comprise any taste-modifying polypeptide as disclosed herein, either recombinantly produced or naturally produced. For example, a yogurt product of the disclosure may comprise one or more of: brazzein, pentadin, thaumatin, monellin, mabinlin, miraculin, and curculin; or any functional fragment thereof; or any mutant thereof.

In some cases, the taste-modifying polypeptide may be added directly to or mixed in to the yogurt formulation. In other cases, the taste-modifying polypeptide may be in a separate formulation that is to be consumed with the yogurt product. For example, the taste-modifying polypeptide may be formulated as a gel, jelly, compote, or puree. In some cases, the gel, jelly, compote, or puree may be provided as a layer or a film overlaying the yogurt product (e.g., a flavored gel layer containing therein a taste-modifying polypeptide, overlaying a yogurt composition). In some cases, the taste-modifying polypeptide may be provided in a separate formulation that accompanies the yogurt product (e.g., provided in a “side-car” of a yogurt package, e.g., as a gel, jelly, compote, or puree). In some cases, the taste-modifying polypeptide may be provided as a coating or a film on the surface of a utensil (e.g., a spoon), that may be supplied or sold to be used with the yogurt product.

A yogurt product comprising taste-modifying polypeptides can comprise any suitable amount of taste-modifying polypeptide. For example, a yogurt product may comprise about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, or about 0.500 mg of purified taste-modifying polypeptide. A yogurt product comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of purified polypeptide.

A non-limiting example of a yogurt product comprising a miraculin is disclosed in Example 18. In some cases, the yogurt product may comprise recombinant miraculin. In some cases, the yogurt product may comprise miraculin purified from a natural source (e.g., plant). In some cases, the amount of purified miraculin may be from about 0.0001% w/w to about 0.0050% w/w. For example, the amount of purified miraculin in a yogurt formulation may be about 0.0001% w/w, about 0.0005% w/w, about 0.0010% w/w, about 0.0015% w/w, about 0.0020% w/w, about 0.0025% w/w, about 0.0030% w/w, about 0.0035% w/w, about 0.0040% w/w, about 0.0045% w/w, or about 0.0050% w/w. In some cases, the yogurt product may comprise miracle berry powder. In some cases, the amount of miracle berry powder may be from about 0.05% w/w to about 5% w/w. For example, the amount of miracle berry powder in a yogurt formulation may be about 0.05% w/w, 0.1% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, or about 5.0% w/w.

### G. Frozen Formulations

In some aspects, a composition disclosed herein comprising a taste-modifying polypeptide may be provided as a frozen formulation. In some cases, a frozen formulation of the disclosure may comprise a purified taste-modifying polypeptide. In some cases, the purified taste-modifying polypeptide may comprise a recombinantly expressed taste-modifying polypeptide as disclosed herein. In some cases, the purified taste-modifying polypeptide may comprise a taste-modifying polypeptide purified from a natural source (e.g., a plant source) as disclosed herein. In some aspects, a frozen formulation of the disclosure may comprise an unpurified taste-modifying polypeptide from a natural source (e.g., whole berry powder). In some cases, a frozen formulation of the disclosure may comprise any taste-modifying polypeptide as disclosed herein, either recombinantly produced or naturally produced. For example, a frozen formulation of the disclosure may comprise one or more of: brazzein, pentadin, thaumatin, monellin, mabinlin, miraculin, and curculin; or any functional fragment thereof; or any mutant thereof.

In some aspects, the frozen formulation may be a frozen food product. In some cases, the frozen food product may be ice cream or an ice cream product. In some cases, the frozen food product may be sorbet. In some cases, the frozen food product may be a frozen popsicle. In some cases, the taste-modifying polypeptide may be added directly to or mixed in to the frozen food product formulation. In some cases, the taste-modifying polypeptide may be provided as a separate formulation for consumption with a frozen food product. In some cases, the separate formulation may be a coating or a layer on the surface of a frozen food product.

In a particular example, the frozen food product may be a frozen popsicle. In some cases, the taste-modifying polypeptide may be provided directly to the frozen popsicle. In some cases, the taste-modifying polypeptide may be provided as a gel, jelly, compote, puree, or powder. In some cases, the gel, jelly, compote, puree, or powder may coat the surface of the frozen popsicle (e.g., at the top portion of the popsicle, or covering the entire surface of the popsicle). In some cases, the gel, jelly, compote, puree, or powder may coat or cover the about 1 to about 50% of the popsicle. For example, the gel, jelly, compote, puree, or powder may coat or cover about 1%, about 5%, about 10%, about 15%, about 20%, about 25% about 30%, about 35%, about 40%, about 45%, or about 50% of the popsicle

A frozen food product comprising taste-modifying polypeptides can comprise any suitable amount of taste-modifying polypeptide. For example, a frozen food product may comprise about 0.001 mg, about 0.002 mg, about 0.003 mg, about 0.004 mg, about 0.005 mg, about 0.006 mg, about 0.007 mg, about 0.008 mg, about 0.009 mg, about 0.010 mg, about 0.015 mg, about 0.020 mg, about 0.025 mg, about 0.030 mg, about 0.035 mg, about 0.040 mg, about 0.045 mg, about 0.050 mg, about 0.055 mg, about 0.060 mg, about 0.065 mg, about 0.070 mg, about 0.075 mg, about 0.080 mg, about 0.085 mg, about 0.090 mg, about 0.095 mg, about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, about 0.400 mg, about 0.425 mg, about 0.450 mg, about 0.475 mg, or about 0.500 mg of purified taste-modifying polypeptide. A frozen food product comprising taste-modifying polypeptides can contain, for example, between about 0.100 mg and about 0.500 mg, between about 0.125 mg and about 0.475 mg, between about 0.150 mg and about 0.450 mg, between about 0.175 mg and about 0.425 mg, between about 0.200 mg and about 0.400 mg, between about 0.225 mg and about 0.375 mg, between about 0.250 mg and about 0.350 mg, or between about 0.275 mg and about 0.325 mg of taste-modifying polypeptide.

Non-limiting examples of popsicles or a popsicle coating (e.g., puree) comprising miraculin are disclosed in Examples 15-17 and 21. In some cases, the popsicle or popsicle coating may comprise recombinant miraculin. In some cases, the popsicle or popsicle coating may comprise miraculin purified from a natural source (e.g., plant). In some cases, the amount of purified miraculin may be from about 0.0001% w/w to about 0.0050% w/w. For example, the amount of purified miraculin in a popsicle or popsicle coating may be about 0.0001% w/w, about 0.0005% w/w, about 0.0010% w/w, about 0.0015% w/w, about 0.0020% w/w, about 0.0025% w/w, about 0.0030% w/w, about 0.0035% w/w, about 0.0040% w/w, about 0.0045% w/w, or about 0.0050% w/w. In some cases, the popsicle or popsicle coating may comprise miracle berry powder. In some cases, the amount of miracle berry powder may be from about 0.1% w/w to about 5% w/w. For example, the amount of miracle berry powder in a popsicle may be about 0.1% w/w, about 0.5% w/w, about 1.0% w/w, about 1.5% w/w, about 2.0% w/w, about 2.5% w/w, about 3.0% w/w, about 3.5% w/w, about 4.0% w/w, about 4.5% w/w, or about 5.0% w/w. In some cases, a popsicle or popsicle coating may comprise both purified miraculin and miracle berry powder.

In a particular aspect, a popsicle formulation of the disclosure may include one of more of the following ingredients: fruit puree (e.g., mango puree, passion fruit puree, strawberry puree, pineapple puree, etc.), water, salt, orange flower water, rose water, juice (e.g., lime juice, lemon juice), natural flavors, and the taste-modifying polypeptide.

### H. Packaging

In some aspects, compositions comprising oral dosages herein can be packaged into a container. In some cases, compositions comprising oral dosages herein may be packaged in a container selected from the group consisting of: a box, a tube, a jar, a vial, a bag, a pouch, a drum, a bottle, and a can. The container may contain information describing directions for consuming the composition.

### EXAMPLES

Various aspects of the disclosure are further illustrated by the following non-limiting examples.

### Example 1: Miraculin Expression and Purification from K. lactis

To express miraculin in K. lactis, a high-copy K. lactis colony was first generated. The miraculin gene was codon optimized for K. lactis. The gene was then synthesized and cloned into pKLAC2 vector (New England Biolabs N3742S). The synthesized plasmid (2 μg) was digested with SacII restriction enzyme (New England Biolabs) and the expression cassette was gel purified. The expression cassette was transformed into K. lactis GG799 competent cells (New England Biolabs C1001S) according to the manufacturer's instruction manual. High copy colonies were screened by colony PCR using integration primer 2 and 3 from the kit (New England Biolab E1000S).

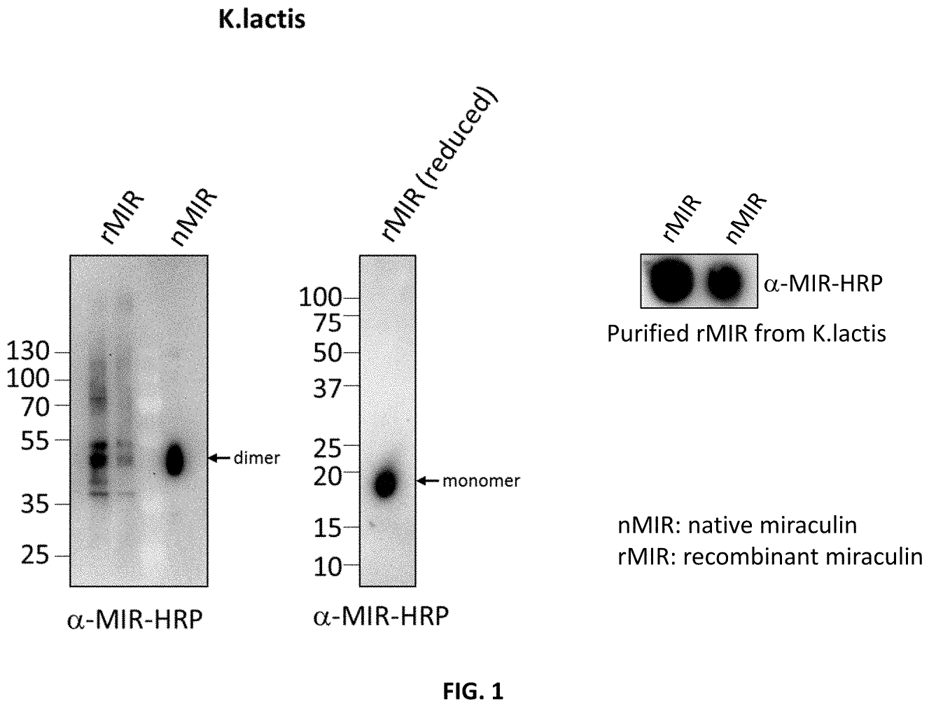

The selected high copy colony was then grown in 2 ml YPGlu (˜pH 5) media for 18 hours at 30° C. with shaking (250 rpm). Next, 1 ml of the overnight culture was inoculated into 50 ml YPGlu (˜pH 5) media and incubated for 18 hours at 30° C. with shaking (250 rpm). 10 ml of the overnight culture was transferred into 500 ml YGU (˜pH 4) media plus 1 mM DTT and incubated for 72 hours at 16° C. with shaking (250 rpm). After 72 hours, the cultures were harvested by centrifugation at 10,000×g for 20 minutes at 4° C. The cell pellet was analyzed by SDS-PAGE and transferred to a PVDF membrane for western blot. Anti-Miraculin antibody was used to confirm presence of miraculin (FIG. 1). The supernatant was collected and the pH was adjusted to pH 4 if necessary. The supernatant was filtered with a 0.22 μm PES filter.

The miraculin was then purified by cation exchange purification. First, a HiPrep SPXL 16/10 column (GE Healthcare Life Sciences 28936540) was washed with 100 ml of milli-Q water. The column was equilibrated with 40 ml of buffer A (20 mM sodium citrate, pH 4) at 5 ml/min. Next, the filtrated culture supernatant was loaded onto the equilibrated column at 5 ml/min. Next, the column was washed with buffer A at 5 ml/min until the OD280 was below 100 mAU. Then, the column was washed with 100 ml of 100 mM NaCl in 20 mM sodium citrate (10% of buffer B, (1 M NaCl in 20 mM sodium citrate, pH 4)). Next, miraculin was eluted with NaCl gradient for 40 minutes (at t=0 minutes A:B=90:10, at t=40 minutes A:B=0:100 (5 ml/min)). 5 ml fractions were collected for 200 ml and the peak appears at 40˜60% gradient. (400 mM˜600 mM NaCl). Elution fractions were analyzed by dot blot. Anti-Miraculin antibody was used to confirm presence of miraculin (FIG. 1).

After purification, the protein was concentrated and subjected to buffer exchange. Fractions containing the peak were pooled and concentrated with Amicon ultra-15 centrifugal filter units with ultracel-10 membrane (Millipore Sigma UFC901024). Per the manufacturer's instructions, buffer exchange was performed with ice-cold bottle water (8 ml to ˜2 ml for each cycle, 6 cycles with water). The sample was adjusted to a concentration of OD280=0.6 (in water).

The following culture media and solutions were used.

A. 40% Glucose solution
Add 400 g glucose to 500 ml milli-Q water.
Dissolve by stirring, warm at 50° C. and adjust the volume to 1 L with milli-Q water.
Filter sterilize with 0.22 μm filter.

B. YPGlu medium
Dissolve 10 g Yeast extract and 20 g Bacto Peptone in 950 ml of milli-Q water.
Adjust the pH to 5.0 with HCl.
Autoclave for 20 minutes at 121° C. Let cool to room temperature.
Aseptically add 50 ml of sterile 40% Glucose.

C. YGU medium
Dissolve 12 g yeast extract, 26 g galactose, and 5 g urea in 800 ml of milli-Q water.
Adjust the pH to 4.0 with HCl.
Bring final volume to 1 L with milli-Q water.
Filter sterilize with 0.22 μm filter. (Do not autoclave urea)

D. 20 mM sodium citrate, ˜pH 4
Dissolve 3.84 g citric acid in 900 ml milli-Q water.
Adjust pH to 4.0 with NaOH.
Bring final volume to 1 L with milli-Q water.

E. 1 M NaCl in 20 mM sodium citrate, ˜pH 4
Dissolve 3.84 g citric acid and 58.4 g NaCl in 900 ml milli-Q water.
Adjust pH to 4.0 with NaOH.
Bring final volume to 1 L with milli-Q water.

### Example 2: Brazzein Expression and Purification from K. lactis

To express brazzein in K. lactis, a high-copy K. lactis colony was first generated. The brazzein (dBRZ, Brazzein-1) gene was codon optimized for K. lactis. The gene was then synthesized and cloned into pKLAC2 vector (New England Biolabs N3742S). The synthesized plasmid (2 μg) was digested with SacII restriction enzyme (New England Biolabs) and the expression cassette was gel purified. The expression cassette was transformed into K. lactis GG799 competent cells (New England Biolabs C1001S) according to the manufacturer's instruction manual. High copy colonies were screened by colony PCR using integration primer 2 and 3 from the kit (New England Biolab E1000S).

The selected high copy colony was then grown in 2 ml YPGlu (˜pH 5) media for 18 hours at 30° C. with shaking (250 rpm). Next, 1 ml of the overnight culture was inoculated into 50 ml YPGlu (˜pH 5) media and incubated for 18 hours at 30° C. with shaking (250 rpm). 10 ml of the overnight culture was transferred into 500 ml YPGal (˜pH 5) media and incubated for 96 hours at 30° C. with shaking (250 rpm). After 96 hours, the cultures were harvested by centrifugation at 10,000×g for 20 minutes at 4° C. The supernatant was collected and the pH was adjusted to pH 4 with citric acid. The supernatant was filtered with a 0.22 μm PES filter.

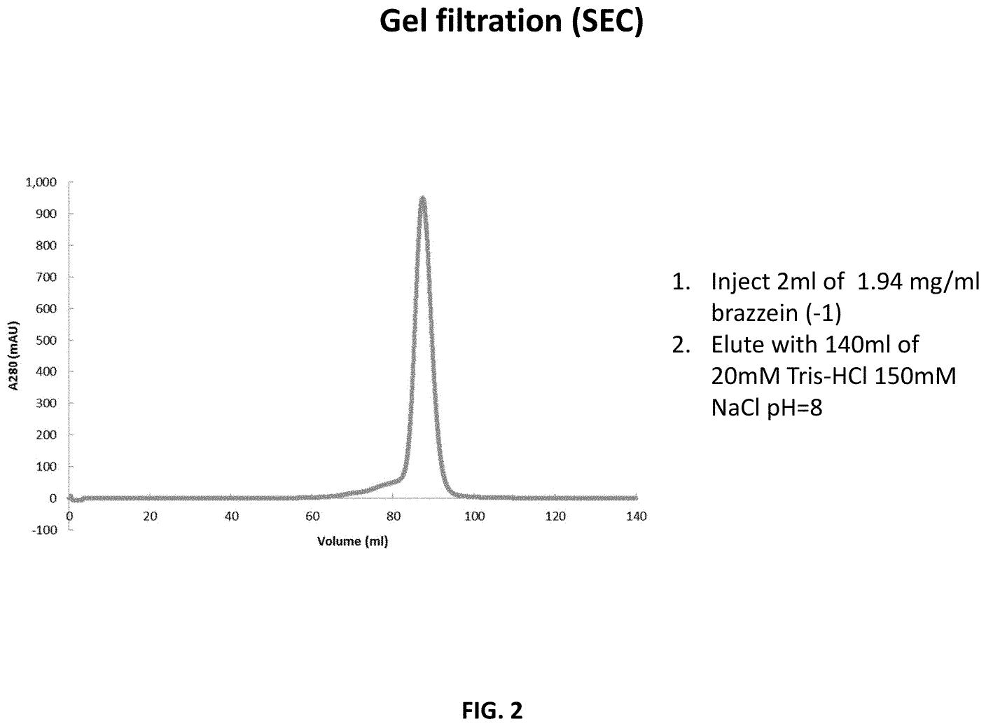

The brazzein was then purified by cation exchange purification. First, a HiPrep SPXL 16/10 column (GE Healthcare Life Sciences 28936540) was washed with 100 ml of milli-Q water. The column was equilibrated with 40 ml of buffer A (20 mM sodium citrate, pH 4) at 5 ml/min. Next, the filtrated culture supernatant was loaded onto the equilibrated column at 5 ml/min. Next, the column was washed with buffer A at 5 ml/min until the OD280 was below 100 mAU. Then, the column was washed with 100 ml of 100 mM NaCl in 20 mM sodium citrate (10% of buffer B, (1 M NaCl in 20 mM sodium citrate, pH 4)). Next, brazzein was eluted with NaCl gradient for 40 minutes (at t=0 minutes A:B=90:10, at t=40 minutes A:B=0:100 (5 ml/min)). 5 ml fractions were collected for 200 ml and the peak appears at 40˜60% gradient (400 mM˜600 mM NaCl). Protein was further purified using HiPrep 16/60 S-100 gel filtration (GE Healthcare Life Sciences 17116501) (FIG. 2).

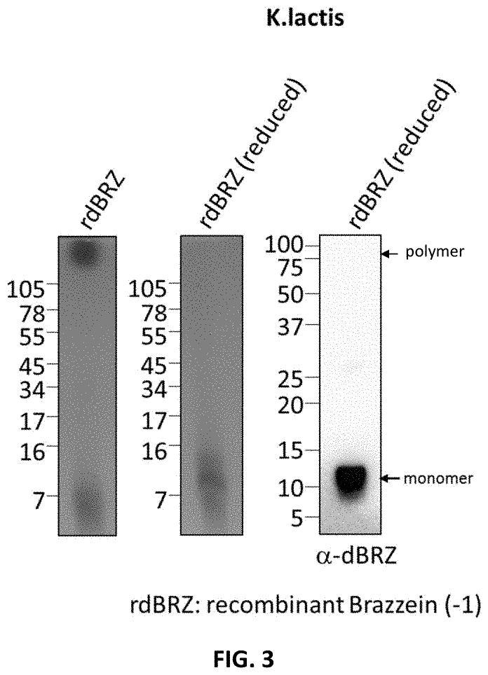

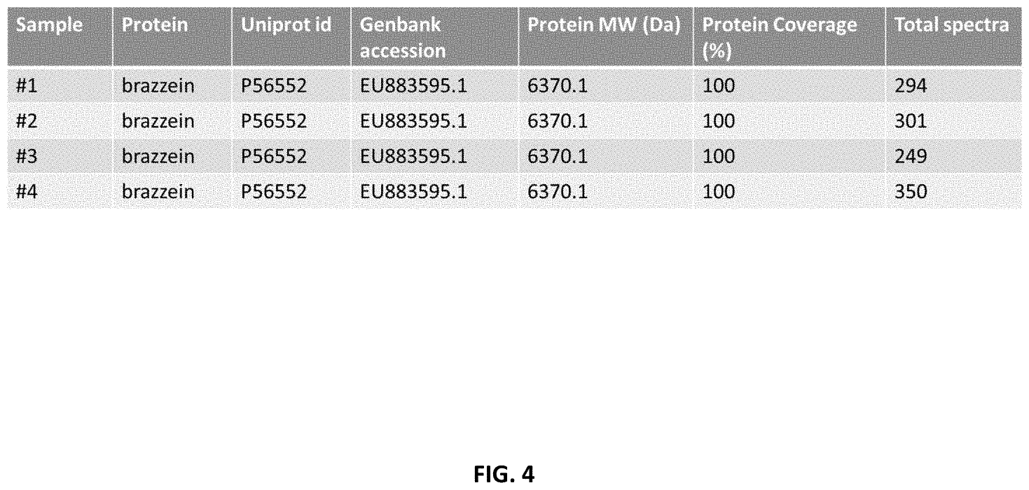

After purification, the protein was concentrated and subjected to buffer exchange. Fractions containing the peak were pooled and concentrated with Amicon ultra-15 centrifugal filter units with ultracel-3 membrane (Millipore Sigma UFC900324). Per the manufacturer's instructions, buffer exchange was performed with ice-cold bottle water (8 ml to ˜2 ml for each cycle, 6 cycles with water). The sample was adjusted to a concentration of OD280=0.7 (in water). Proteins were separated by SDS-PAGE and transferred to a PVDF membrane for western blot. Anti-Brazzein antibody was used to confirm presence of Brazzein (FIG. 3). Liquid chromatography tandem mass spectrometry (LC-MS/MS) protein identification analysis was used to confirm presence of Brazzein (FIG. 4)

The following culture media and solutions were used.

A. 40% Glucose solution or 40% Galactose solution
Add 400 g glucose or galactose to 500 ml milli-Q water.
Dissolve by stirring, warm at 50° C. and adjust the volume to 1 L with milli-Q water.
Filter sterilize with 0.22 μm filter.

B. YPGlu & YPGal medium
Dissolve 10 g Yeast extract and 20 g Bacto Peptone in 950 ml of milli-Q water.
Adjust the pH to 5.0 with HCl.
Autoclave for 20 minutes at 121° C. Let cool to room temperature.
Aseptically add 50 ml of sterile 40% glucose for YPGlu or 40% galactose for YPGal.

C. 20 mM sodium citrate, ˜pH 4
Dissolve 3.84 g citric acid in 900 ml milli-Q water.
Adjust pH to 4.0 with NaOH.
Bring final volume to 1 L with milli-Q water.

D. 1 M NaCl in 20 mM sodium citrate, ˜pH 4
Dissolve 3.84 g citric acid and 58.4 g NaCl in 900 ml milli-Q water.
Adjust pH to 4.0 with NaOH.
Bring final volume to 1 L with milli-Q water.

### Example 3: Miraculin Expression from P. pastoris

To express miraculin in P. pastoris, a high-copy P. pastoris colony was first generated. The miraculin gene was codon optimized for P. pastoris. The gene was then synthesized and cloned into a modified pPIC9K vector (Thermo Fisher Scientific V17520) with GAP promoter. The synthesized plasmid (2 μg) was linearized with SalI restriction enzyme (New England Biolabs) and gel purified. The linearized plasmid was transformed into P. pastoris GS115 competent cells (Thermo Fisher Scientific C18100) according to the manufacturer's instruction manual (Thermo Fisher Scientific K171001). High copy colonies were screened by YPD plates with 4 mg/ml geneticin.

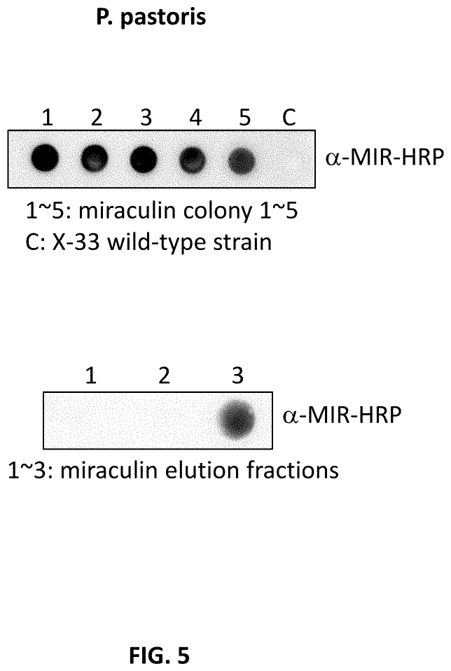

The selected high copy colony was then grown in 4 ml MGY (˜pH 4) media at 28° C. with shaking (250 rpm). Next, 1 ml of the culture was harvested every 24 hours for dot blot analysis to confirm protein expression and secretion. Samples were harvested by centrifugation at 14,000 rpm for 2 minutes. The supernatant was collected and loaded onto nitrocellulose membrane for dot blot. Miraculin expression in Pichia culture supernatant was confirmed (FIG. 5).

The selected high copy colony was then grown in 4 ml MGY (˜pH 4) media for 18 hours at 28° C. with shaking (250 rpm). Next, 3 ml of the overnight culture was inoculated into 500 ml MGY (˜pH 4) media and incubated for 48 hours at 28° C. with shaking (250 rpm). After 48 hours, the cultures were harvested by centrifugation at 10,000×g for 20 minutes at 4° C. The supernatant was collected and the pH was adjusted to pH 4, if necessary. The supernatant was filtered with a 0.22 μm PES filter.

The miraculin was then purified by cation exchange purification. First, a HiPrep SPXL 16/10 column (GE Healthcare Life Sciences 28936540) was washed with 100 ml of milli-Q water. The column was equilibrated with 40 ml of buffer A (20 mM sodium citrate, pH 4) at 5 ml/min. Next, the filtrated culture supernatant was loaded onto the equilibrated column at 5 ml/min. Next, the column was washed with buffer A at 5 ml/min until the OD280 was below 100 mAU. Then, the column was washed with 100 ml of 100 mM NaCl in 20 mM sodium citrate (10% of buffer B, (1 M NaCl in 20 mM sodium citrate, pH 4)). Next, miraculin was eluted with NaCl gradient for 40 minutes (at t=0 minutes A:B=90:10, at t=40 minutes A:B=0:100 (5 ml/min)). 5 ml fractions were collected for 200 ml and the peak appeared at 40˜60% gradient (400 mM˜600 mM NaCl). Elution fractions were analyzed by dot blot. Anti-Miraculin antibody was used to confirm presence of miraculin (FIG. 5)

After purification, the protein was concentrated and subjected to buffer exchange. Fractions containing the peak were pooled and concentrated with Amicon ultra-15 centrifugal filter units with ultracel-10 membrane (Millipore Sigma UFC901024). Per the manufacturer's instructions, buffer exchange was performed with ice-cold bottle water (8 ml to ˜2 ml for each cycle, 6 cycles with water). The sample was adjusted to a concentration of OD280=0.6 (in water).

### Example 4: Brazzein Expression from P. pastoris

To express brazzein in P. pastoris, a high-copy P. pastoris colony was first generated. The brazzein gene was codon optimized for P. pastoris. The gene was then synthesized and cloned into a modified pPIC9K vector (Thermo Fisher Scientific V17520) with GAP promoter. The synthesized plasmid (2 μg) was linearized with SalI restriction enzyme (New England Biolabs) and gel purified. The linearized plasmid was transformed into P. pastoris GS115 competent cells (Thermo Fisher Scientific C18100) according to the manufacturer's instruction manual (Thermo Fisher Scientific K171001). High copy colonies were screened by YPD plates with 4 mg/ml geneticin.

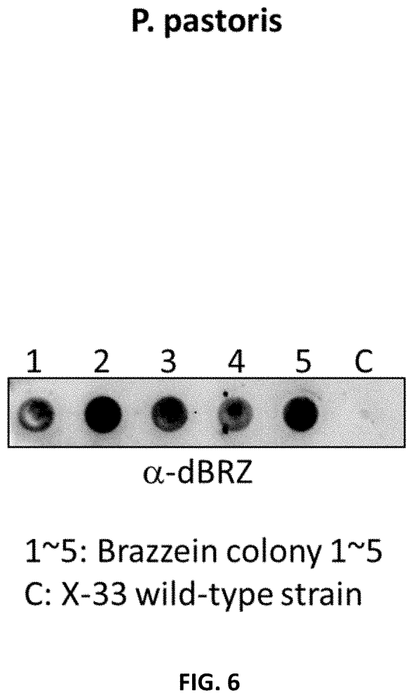

The selected high copy colony was then grown in 4 ml MGY (˜pH 4) media at 28° C. with shaking (250 rpm). Next, 1 ml of the culture was harvested every 24 hours for dot blot analysis to confirm protein expression and secretion. Samples were harvested by centrifugation at 14,000 rpm for 2 minutes. The supernatant was collected and loaded onto nitrocellulose membrane for dot blot. Brazzein expression in Pichia culture supernatant was confirmed (FIG. 6). Large scale expression will be performed according to the manufacturer's instruction manual (Thermo Fisher Scientific K171001).

### Example 5: Miraculin Expression from E. coli

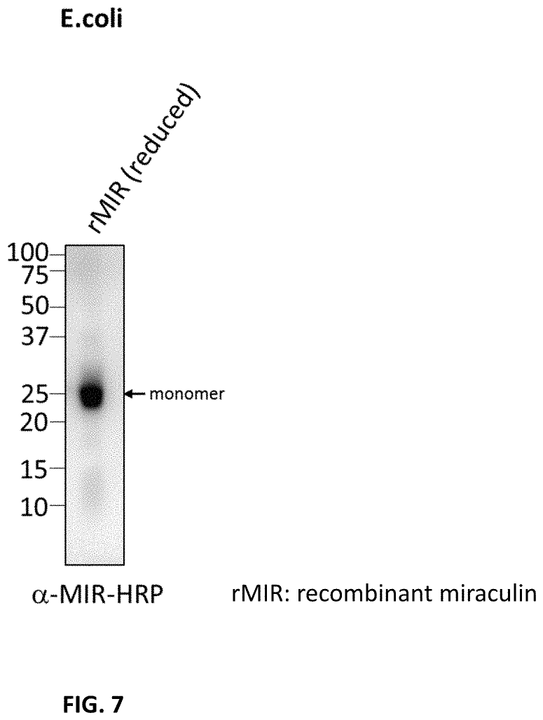

The open reading frame of Miraculin was first cloned into pET-28a(+) vector (Novagen). BL21-DE3 competent cells (New England Biolabs C25271) were transformed according to manufacturer specifications. 100 μL of transformation was added to a 5 mL culture of LB with appropriate antibiotics. Culture was grown overnight at 37° C. A 100 ml culture was inoculated from the starter culture and allowed to grow to a starting OD₆₀₀ of 0.6 before inducing with 500 μM IPTG. Induction took place for 24 hours at 16° C. 1 mL of culture was harvested and pelleted at 14,000×g. 100 μL of SDS sample buffer with 50 mM TCEP was added to the cell pellet. Proteins were separated by SDS-PAGE and transferred to a PVDF membrane for western blot. Anti-Miraculin antibody was used to confirm presence of miraculin (FIG. 7).

### Example 6: Brazzein expression from E. coli

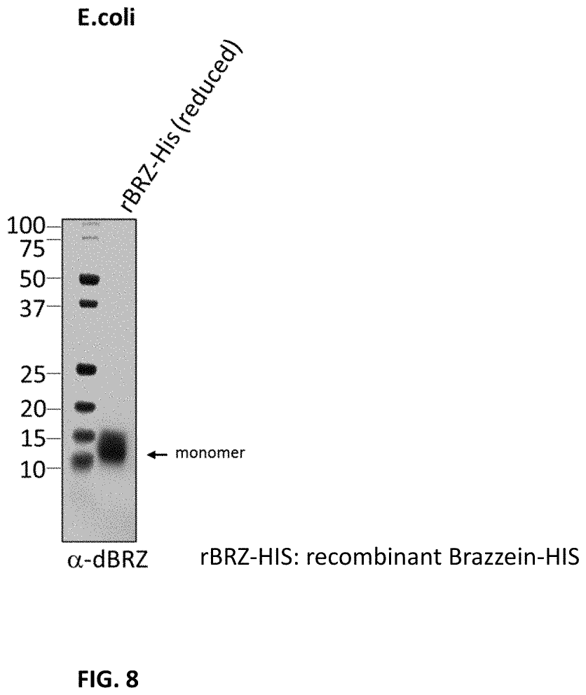

The open reading frame of Brazzein was first cloned into pET-28a(+) vector (Novagen). BL21-DE3 competent cells (New England Biolabs C25271) were transformed according to manufacturer specifications. 100 μL of transformation was added to a 5 mL culture of LB with appropriate antibiotics. The culture was grown overnight at 37° C. 100 mL of culture was inoculated from the starter culture and allowed to grow to a starting OD₆₀₀ of 0.6 before inducing with 500 μM IPTG. The culture was grown for 3 hours at 28° C. The culture was centrifuged at 14,000×g for 15 minutes. Cells were disrupted by vortexing with glass beads while in a 4° C. fridge. 100 mL extraction buffer (50 mM NaCl, 20 mM Sodium Citrate, 50 mM Ascorbic acid, 5% polyvinylpolypyrrolidone, 1 mM phenylmethylsulfonyl fluoride, 10 μM Leupeptin) was added and cell debris was removed by centrifugation at 14,000×g. Supernatant was filtered through a 0.22 μm PES membrane before loading onto a calibrated HiTrap SP FF column (GE 17515701). Unbound proteins were removed by washing with 20 mM Sodium Citrate until A280 read below 100 mAU. Bound proteins were eluted with a salt-based gradient (from 0 mM to 1 M NaCl) over 100 mL. Fractions were concentrated and Proteins were separated by SDS-PAGE and transferred to a PVDF membrane for western blot. Anti-Brazzein antibody was used to confirm presence of Brazzein (FIG. 8).

### Example 7: Thin Film

In this example, a taste-modifying polypeptide comprising miraculin was formulated into an oral thin film.

TABLE 7
Formulation of oral thin film (~8 servings)

| Ingredients | Amount | |
|---|---|---|
| Lycoat ® RS780 | 0.56 | g |
| Hydroxypropyl methylcellulose (HPMC, 2%) | 1.2 | ml |
| Red food color (FD&C Red # 40) | 6 | μl |
| Blended solution of citric acid and ascorbic acid | 1.5 | ml |
| Vegetable glycerin (4%) | 1.5 | ml |
| Purified miraculin protein (0.5 mg/ml) | 4 | ml |
| H₂O | 11.8 | ml |

Lycoat RS780 is a pre-gelatinized modified pea starch obtained from ROQUETTE America Inc. The 2% hydroxypropyl methylcellulose (HPMC, Methocel F50 Food grade) was made by dissolving 2 g of HPMC in 100 ml H₂O. The blended solution of citric acid and ascorbic acid was made by dissolving 0.46 g citric acid and 0.4 g ascorbic acid in 50 ml H₂O. The 4% vegetable glycerin (USP Food grade) was made by dissolving 4 g vegetable glycerin in 100 ml H₂O.

The following steps were implemented to make oral thin films comprising miraculin. First, 0.56 g of Lycoat® RS780 was dissolved in water and heated for about 5-7 minutes on a hot plate for better dissolution. After at least an hour with continuous stirring, 1.2 ml of 2% HPMC was added to the Lycoat® RS780 solution and mixed for at least an hour. Then, 6 μl of red food color (FD&C Red #40) was added to the polymer solution and mixed for another hour. Next, 1.5 ml of the blended solution of citric acid and ascorbic acid was added to the polymer solution and mixed for at least an additional hour. Then, 1 ml of 4% vegetable glycerin was added to the film solution and mixed for at least an hour. Next, 4 ml of the purified miraculin protein liquid (at a concentration of 0.5 mg/ml) was added to film solution and mixed for at least 40 minutes. Then, 20 ml of the film solution was poured onto a film mold comprising a square petri dish (9 cm×9 cm) and the film was formed by drying at room temperature with forced-air flow until the film reached a constant weight (approximately 8 hours). In preferred examples, the final film solution is bubble-free before casting on the film mold. Finally, the dried film was peeled off from the petri dish and cut into 3.2 cm×2 cm strips and stored in a sealed package at room temperature.

The resulting thin films in this example each contained about 0.250 mg of purified miraculin. Thin film preparations can contain more or less miraculin. For example, thin film preparations can contain about 0.100 mg, about 0.125 mg, about 0.150 mg, about 0.175 mg, about 0.200 mg, about 0.225 mg, about 0.250 mg, about 0.275 mg, about 0.300 mg, about 0.325 mg, about 0.350 mg, about 0.375 mg, or about 0.400 mg of miraculin.

### Example 8: Edible Gel

In this example, a taste-modifying polypeptide comprising miraculin was formulated into an edible gel.

TABLE 8
Formulation of jelly (~24 servings)

| Ingredients | Amount | |
|---|---|---|
| Miracle Fruit Powder | 9 | g |
| Cranberry juice | 140 | ml |
| Unflavored gelatine | 18 | g |
| Blended solution of citric acid and ascorbic acid | 15 | ml |
| H₂O | 155 | ml |

Pure Miracle Fruit Powder (powdered berry from Synsepalum dulcificum) was purchased from Miracle Fruit Farm, FL. The blended solution of citric acid and ascorbic acid was made by dissolving 0.46 g citric acid and 0.4 g ascorbic acid in 50 ml H₂O.

The following steps were implemented to make edible gels comprising miraculin. The fruit powder (9 g) was mixed with 15 ml of the blended solution of citric acid and ascorbic acid and with 65 ml H₂O until the powder was evenly distributed in the liquid solution. Next, 18 g of unflavored gelatine was dissolved in 90 ml of heated H₂O. Then, 140 ml of cold cranberry juice was added to unflavored gelatin solution and mixed well. The fruit powder solution was then added to the mixture of gelatin and cranberry juice, and the resulting mixture was mixed well. The final solution was poured into a jelly mold and stored in a refrigerator until firm for at least 3 hours. The firmed jelly was finally cut into small cubes and stored in an air-tight container in a refrigerator.

The resulting edible gels in this example each contained about 0.375 g of fruit powder. Fruit powder, in addition to polypeptide, can comprise other berry components, such as pulp and skin. Fruit powder may contain at most 50% w/w of miraculin (e.g., at most about 45%, about 40%, about 35%, about 30%, about 25%, about 20%, about 15%, about 10%, about 5%, about 4%, about 3%, about 2%, about 1%, about 0.1%, or less). An edible gel can contain less than about 200 mg, about 100 mg, about 50 mg, about 10 mg, about 5 mg, about 4 mg, about 3 mg, about 2 mg, about 1 mg, about 0.900 mg, about 0.800 mg, about 0.700 mg, about 0.600 mg, about 0.500 mg, about 0.450 mg, about 0.400 mg, about 0.350 mg, about 0.300 mg, about 0.250 mg, about 0.200 mg, about 0.150 mg, or about 0.100 mg of miraculin polypeptide.

### Example 9: Fruit Bites (8 Servings)

In this example, a taste-modifying polypeptide comprising miraculin was formulated into fruit bites.

TABLE 9
Formulation of fruit bites

| Ingredients | Amount | |
|---|---|---|
| Tapioca Starch | 1.39 | g |
| Hydroxypropyl methylcellulose (HPMC, 2%) | 5 | ml |
| Polysorbate 20 (Food grade) | 5 | ml |
| Blended solution of citric acid and ascorbic acid | 5 | ml |
| Miracle Fruit Powder | 5 | g |
| Vegetable glycerin (4%) | 8 | ml |
| H₂O | 22 | ml |

The 2% hydroxypropyl methylcellulose (HPMC, Methocel F50 Food grade) was made by dissolving 2 g of HPMC in 100 ml H₂O. The blended solution of citric acid and ascorbic acid was made by dissolving 0.46 g citric acid and 0.4 g ascorbic acid in 50 ml H₂O. The Polysorbate 20 (USP, Kosher Pure, Food Grade) solution was made by mixing 1.825 g Polysorbate 20 with 50 ml H₂O. The 4% vegetable glycerin (USP Food grade) was made by dissolving 4 g vegetable glycerin in 100 ml H₂O. Miracle Fruit Powder (powdered berry from Synsepalum dulcificum) was purchased from Miracle Fruit Farm, FL.

The following steps were implemented to make fruit bites comprising miraculin. First, 1.39 g tapioca starch was dissolved in water and heated until it formed gel. Then the tapioca starch solution was stirred continually for an hour. Next, 5 ml of 2% HPMC was added to the starch solution and mixed for at least an hour. Then, 5 ml of Polysorbate 20 solution was added and the solution was mixed for another hour. Next, 5 ml of the blended solution of citric acid and ascorbic acid was added to the mixture and mixed for at least an hour. Then, 8 ml of 4% vegetable glycerin was added to the solution and mixed for at least an hour. Next, 5 g fruit powder was added to the solution and the solution was mixed for at least 40 minutes. Then, 45 ml of the final solution was poured onto a mold comprising a square petri dish (9 cm×9 cm). The fruit bites were formed by drying the solution at room temperature with forced-air flow until the mixture on the petri dish reached a constant weight. Finally, the mixture was peeled off of the petri-dish and the resulting fruit product was rolled up and cut into 8 pieces. The fruit bites were stored in an air-tight container in a refrigerator.

The resulting fruit bites in this example each contain about 0.625 g of fruit powder. Fruit powder, in addition to polypeptide, can comprise other berry components, such as pulp and skin. Fruit powder may contain at most about 50% w/w of miraculin (e.g., at most about 45%, about 40%, about 35%, about 30%, about 25%, about 20%, about 15%, about 10%, about 5%, about 4%, about 3%, about 2%, about 1%, about 0.1%, or less). A fruit chew can contain less than about 350 mg, about 300 mg, about 250 mg, about 200 mg, about 150 mg, about 100 mg, about 50 mg, about 10 mg, about 5 mg, about 4 mg, about 3 mg, about 2 mg, about 1 mg, about 0.900 mg, about 0.800 mg, about 0.700 mg, about 0.600 mg, about 0.500 mg, about 0.450 mg, about 0.400 mg, about 0.350 mg, about 0.300 mg, about 0.250 mg, about 0.200 mg, about 0.150 mg, or about 0.100 mg of miraculin polypeptide.

### Example 10: Extraction of miraculin from miracle berries

In this example, miraculin was extracted from miracle berries. First, 600 g of berry (˜356 berries) was weighed out. A solution of 0.5M NaCl+5 mM EDTA (29.2 g NaCl+10 ml 0.5 M EDTA in 1 L MilliQ H₂O) was used at a 1:1 ratio to separate berry pulp from berry seeds and skins (˜600 ml of solution for 600 g of berries).

Berries were processed in 300 g batches. First, half of the first batch of berries was added to a large plastic 1 liter (1 L) beaker. Then, 150 mL of ice-cold 0.5 M NaCl+EDTA and 0.35 g ascorbate was added to the berries in the beaker. The berries were mixed with a handmixer while the beaker was on ice. The remaining half of the berries was added to the beaker when a suitable amount of separation from pulp and seed was observed. The berries were further mixed with the handmixer until a suitable amount of separation was observed for the entire batch of berries. The mixture was placed into a salad spinner and the salad spinner was used to separate liquid from seeds and skins. The liquid was collected into a separate container and placed on ice.

The skins and seeds in the salad spinner were placed back into the beaker and an additional 150 mL of 0.5 M NaCl was added to the beaker. The berry seeds and skins were mixed by handmixer to obtain pulp that remained on the seeds. The mixture was again placed into the salad spinner to separate the liquid from the seeds. The liquid was collected into a separate container and placed on ice. The mixture was spun 1-2 more times to collect remaining liquid from the seeds and skins. From 300 g of whole berry, an average of 103 g of berry extract (not including weight from NaCl added), an average of 61.2 g berry skins, and an average of 136 g of total seeds was obtained. The total collected volume was approximately 400 ml˜450 ml of total berry extract. This was repeated for the second batch of berries to yield a total of approximately 800 ml˜900 ml of berry extract from ˜600 g of berries.

Ascorbate (0.704 g) was added to the collected 800 ml˜900 ml of berry extract (˜5). The berry extract was blended by a food processor using 10 second pulses for 6 pulses. There was a 10 second pause between each 10 second pulse. The berry extract solution was then placed into 250 mL centrifuge bottles and centrifuged for 1 hour at 14,000 rpm at 4° C.

During centrifugation, the pump and ultrafiltration/diafiltration (UFDF) filter were rinsed with MilliQ H₂O (3×500 ml), and then with dialysis buffer one time. The pump was operated at 600 ml/minute with 10-15 psi at rear pressure gauge.

After centrifugation, the berry extract was filtered under vacuum through 70 mm Whatman #3 filter paper into a pre-chilled filter flask on ice. The filtered berry extract was then loaded into UFDF using a 500 ml bottle on ice. The extract was pumped through at 600 ml/minute and pressure at about 25˜30 psi max pressure reading. After loading the extract, a tube was used to drip ice-cold dialysis buffer into the berry extract and maintain the volume at 200˜250 ml to establish a pseudoequilibrium. Dialysis was performed with 2.5 L dialysis buffer. Approximately 300 ml of dialyzed sample was collected from the UFDF system. The pH of the dialyzed sample was adjusted with 1 M Tris base to pH 7.28˜7.29. The pH adjusted, dialyzed sample was then centrifuged at 14,000 rpm for 1 hour at 4° C.

During centrifugation, two chromatography columns were prepared. Each column had 10 ml of Nickel resin slurry and was in a cold box. Each column was washed with 25 ml of water followed by 50 ml of binding buffer. Once washed, the columns were stacked vertically using a ring stand and the output of the top column was connected to the input of the bottom column via tubing.

After centrifugation, the supernatant was filtered with a 0.22 μm Corning PES membrane. The filtered sample (˜300 ml) was loaded onto the connected columns by gravity flow. The sample was allowed to flow through both columns in series. The columns were washed with 150 ml of pH 7.3 loading buffer. Then, the two columns were disconnected and elution fractions were separately collected for each column. Each column was eluted with ˜25 ml of elution buffer, collecting about 1 into each of labeled 2 ml microfuge tubes. (#1˜5 for a 5 ml fraction, #6-15 for 1 ml fractions, #16-25 for 5 ml fractions). All fractions were kept on ice. EDTA was added into eluted samples (peak fractions having OD280>0.2) to a final concentration of 2 mM (4 μl of 0.5 M EDTA into 1 ml). All peak fractions were kept at −20° C.

Then, buffer exchange was performed by UFDF. UFDF (small column) was rinsed 3× with MilliQ H₂O; the pump was run at 300 ml/minute and avg. 10 psi. The pressure was reduced and the pump speed was reduced to 150 ml/minute. MilliQ H₂O was pumped out from the machine. An ice-bath was created for the extract and the extract was placed in a 150 mL beaker. UFDF was run at 300 ml/minute 20 psi on front end and 25 psi on tail end. The liquid volume in beaker was reduced to about 30 mL. Once liquid level reached 30 mL, pressure was reduced and 100 mL MilliQ H₂O was added to the beaker. This was repeated 7× (adding a total of 700 mL of MilliQ H₂O). The liquid level was reduced as low as possible and collected. The pump was reversed to collect more extract from the system.

Another buffer exchange was conducted with Millipore Filter Columns. All samples were kept chilled and/or on ice. First, 15 mL Millipore Filter columns were washed by loading about 7-8 mL of MilliQ H₂O into the filter and spinning for at least 10 minutes at 5000×g at 4° C. The flowthrough and any remaining water in the filter were poured off. Elution samples were pooled and loaded onto the filter columns. Each filter column was filled with a maximum of 7-8 ml of elution samples. Once samples were loaded, the columns were spun for 20 minutes at 5000×g at 4° C. The flowthrough was poured off.

Once all the elution samples had run through the Millipore Filter columns, the columns were washed. The columns were washed by adding water to the top of the label strip (total volume of about 7 mL). The columns were then spun for 15 minutes at 5000×g at 4° C. The flowthrough was poured off after each spin.

### Buffer Recipes

### UFDF Dialysis Buffer

| | MW | Molarity | Weigh out | |
|---|---|---|---|---|
| NaCl | 58.44 | 0.5 | 116.88 | g |
| Citrate | 192.124 | 0.01 | 7.68 | g |

| | | |
|---|---|---|
| Add dH2O up to | 4 | Liters |

Adjust pH to ~3.5 with 10N NaOH
Cool on ice to ~0 degrees before use

Binding/Wash buffer

| | MW | Molarity | Weigh out | |
|---|---|---|---|---|
| NaCl | 58.44 | 0.5 | 29.22 | g |
| Citrate | 192.124 | 0.01 | 1.92 | g |
| Tris base | 121.14 | 0.02 | 2.42 | g |

| | | |
|---|---|---|
| Add dH2O up to | 1 | Liters |

Adjust pH to ~7.3 with 10N NaOH
Cool on ice to ~0 degrees before use

Elution buffer
The same recipe as binding buffer
Adjust pH to ˜4.68
Cool on ice to ˜0 degrees before use

### Example 11: Miraculin Gummy Candies

In this example, a taste-modifying polypeptide comprising miraculin was formulated into fruit-flavored gummy candies.

TABLE 10
Formulation of miraculin gummy candies (79 gummy candies)
Ingredients

| | | | |
|---|---|---|---|
| Part I. | Gelatin, 250 bloom | 25 | g |
| | Water | 40 | ml |
| Part II. | Concentrate apple juice | 44 | ml |
| | Corn starch | 5 | g |
| | Guar gum | 3 | g |
| Part III. | Miracle berry powder | 10 | g |
| | Mixture of citric acid and ascorbic acid | 7.5 | ml |
| | Cranberry juice | 20 | ml |
| | Miraculin solution (0.6 mg/ml) | 8 | ml |
| | Polysorbate 20 | 2.5 | ml |
| | Black berry flavor oil | 0.5 | ml |
| | Citric acid | 3.2 | g |
| | Red food color | 18 | μl |

The blended solution of citric acid and ascorbic acid was made by dissolving 0.46 g citric acid and 0.4 g ascorbic acid in 50 ml H₂O. The Polysorbate 20 (USP, Kosher Pure, Food Grade) solution was made by mixing 1.825 g Polysorbate 20 with 50 ml H₂O. Pure Miracle Fruit Powder (powdered berry from Synsepalum dulcificum) was purchased from Miracle Fruit Farm, FL.

The following steps were implemented to make gummy candies comprising miraculin. First, gelatin and water (Part I) was pre-heated in a hot water bath for 5 minutes. Then the gelatin was whisked into the water until all lumps had disappeared. The gelatin solution was heated over medium-low heat for about 25 minutes continuously, and the solution was stirred until all of the gelatin had melted and had become smooth and glass-like.

Next, 5 g of corn starch was dissolved in 14 ml of concentrate apple juice (Part II). 30 ml of concentrate apple juice was heated over medium-low heat, and the starch mixture was whisked into the warm apple juice. 3 g of guar gum was then added to the mixture and continually stirred until it had formed a jelly texture.

Next, the pre-mixed citric acid and ascorbic acid solution, Polysorbate 20, and cranberry juice was added to the Miracle berry powder and mixed well. Next, citric acid, black berry flavor oil, and red food color were added to the Miracle berry solution (Part III), followed by the miraculin solution.

Next, the gelatin solution (Part I) was added to the starch-guar gum solution (Part II). Once the starch had thickened, the solution was removed from the heat. Next, the Miracle berry solution (Part III) was added to the gelatin and starch mixture, and mixed well. The final liquid mixture was then poured into a gummy mold tray (8×12 inches tray with 5 different kinds of animal molds: a bear, a lion, a monkey, a penguin, and a worm). The mold was stored in the refrigerator until the mixture was firm. Finally, the gummies were removed from the mold, the surfaces of the gummies were sprayed with oil (e.g., avocado oil, other vegetable oil), laid on a tray, and dried under forced-air overnight before packaging.

### Example 12: Sour Gummy Candies

In this example, flavored gummy candies were formulated to be consumed with miraculin gummy candies (see Example 11).

TABLE 11
Formulation of apple gummy candies (79 gummy candies)
Ingredients

| | | | |
|---|---|---|---|
| Part I. | Gelatin, 250 bloom | 25 | g |
| | Water | 40 | ml |
| Part II. | Concentrate apple juice | 50 | ml |
| | Corn starch | 5 | g |
| | Guar gum | 3 | g |
| Part III. | Apple juice | 30 | ml |
| | Citric acid | 12.8 | g |
| | Apple flavor oil | 0.5 | ml |
| | Green liqua-gel food color | 18 | μl |

The following steps were implemented to make apple-flavored sour gummy candies. First, the gelatin and water (Part I) was pre-heated in a hot water bath for 5 minutes. Next, the gelatin was whisked into the water until all the lumps had disappeared. The gelatin solution was heated over medium-low heat for about 25 minutes continuously, and was stirred until all of the gelatin had melted and the solution had become smooth and glass-like.

Next, 5 g of corn starch was dissolved in 15 ml of cold concentrate apple juice (Part II). The remaining 35 ml of concentrate apple juice was heated over medium-low heat. Next, the starch solution was whisked into the warm concentrate apple juice, and then 3 g of guar gum was added to the mixture and continuously stirred until it had formed a jelly texture.

Next, 12.8 g of citric acid, 0.5 ml of apple flavor oil, and 18 μl of green liqua-gel food coloring were added to 30 ml apple juice (Part III), and set aside. In some examples, 15 ml of unsweetened apple source may replace 15 ml of the concentrate apple juice in Part III

Next, the gelatin solution (Part I) was poured into the starch-guar gum solution (Part II). As soon as the starch solution thickened, it was removed from the heat. Next, the mixture of Part III was added into the mixture of gelatin and starch-guar gum solution (Parts I and II) and mixed well. Next, the final liquid was poured into gummy molds and stored in the refrigerator until firm. Finally, the gummies were removed from the mold and the surface of the gummies was sprayed with oil (e.g., avocado oil, or other vegetable oil). The gummies were laid on a rack and dried under forced-air over night before packaging.

### Example 13: Lemon and Lime-Flavored Sour Gummy Candies

In this example, lemon and lime-flavored sour gummy candies were formulated to be consumed with miraculin gummy candies (see Example 11).

TABLE 12
Formulation of lemon and lime gummy candies (79 gummy candies)
Ingredients

| | | | |
|---|---|---|---|
| Part I. | Gelatin, 250 bloom | 25 | g |
| | Water | 40 | ml |
| Part II. | Concentrate orange juice | 50 | ml |
| | Corn starch | 5 | g |
| | Guar gum | 3 | g |
| Part III. | Fresh Lemon juice | 30 | ml |
| | Citric acid | 12.8 | g |
| | Lemon flavor oil | 0.25 | ml |
| | Lime flavor oil | 0.25 | ml |
| | Yellow liqua-gel food color | 18 | μl |

The following steps were implemented to make lemon and lime-flavored sour gummy candies. First, the gelatin and water (Part I) was pre-heated in a hot water bath for 5 minutes. 25 g of gelatin was whisked into 40 ml of water until all of the lumps had disappeared. Next, the gelatin solution was heated over medium-low heat for about 25 minutes, and the mixture was stirred constantly until all of the gelatin had melted and had become smooth and glass-like.

Next, 5 g of corn starch was dissolved in 15 ml of cold concentrate orange juice (Part II). The remaining 35 ml of concentrate orange juice was heated over medium-low heat. Next, the starch solution was whisked into the warm orange juice, and then the 3 g of guar gum was added to the mixture and continuously stirred until it had formed a jelly texture.

Next, 12.8 g of citric acid, 0.25 ml of lemon flavor oil, 0.25 ml of lime flavor oil, and 18 μl of yellow liqua-gel food color were added to 30 ml of fresh lemon juice (Part III), and the mixture was set aside.

Next, the gelatin solution (Part I) was mixed into the starch-guar gum solution (Part II). As soon as the starch mixture thickened, it was removed from the heat. Next, the yellow lemon juice solution (Part III) was added to the mixture of gelatin (Part I) and starch-guar gum (Part II), and mixed well. Next, the final solution was poured into gummy molds and stored in the refrigerator until firm. Next, the gummies were removed from the mold and the surfaces were sprayed with oil (e.g., avocado oil, other vegetable oil). Finally, the gummies were laid on a rack and dried under forced-air over night before packaging.

### Example 14: Freeze-Dried Miraculin Spray

In this example, a taste-modifying polypeptide comprising miraculin was formulated into a freeze-dried miraculin spray.

TABLE 13
Formulation of miraculin spray (16 servings)
Ingredients

| | |
|---|---|
| Purified Miraculin protein (0.6 mg/ml) | 2 ml |
| Ascorbic acid | 1 mg |

The following steps were implemented to make a spray comprising miraculin purified from miracle berries. First, a mini spray bottle (3 ml) was filled with 2 ml of miraculin solution (0.6 mg/ml). Next, the miraculin solution was freeze dried using a lyophilizer (ATR FD 3.0) for 24 hours. Next, 1 mg of ascorbic acid powder was added to the freeze dried miraculin spray bottle. The freeze-dried miraculin powder is stable at room temperature. Next, the freeze-dried miraculin was re-dissolved with 2 ml water before application. There were 32 sprays per 2 ml of solution (2 sprays per serving).

### Example 15: Mango and Passion Fruit Popsicle

In this example, a taste-modifying polypeptide comprising miraculin was formulated into a mango-passion fruit popsicle.

TABLE 14
Formulation of Mango-Passion fruit popsicle (6 servings)
Ingredients

| | | | |
|---|---|---|---|
| Part I. | Mango Puree | 330 | g |
| | Passion Fruit Puree | 90 | g |
| | Water | 30 | g |
| | Orange Flower Water | 0.15 | g |
| | Salt | 0.45 | g |
| Part II. | Strawberry Puree | 18 | g |
| | Miracle berry powder | 15 | g |
| | Miraculin solution (0.5 mg/ml) | 2.4 | ml |

The following steps were implemented to make mango-passion fruit-flavored popsicles comprising miraculin. First, the mango puree (17-19° Brix), passion fruit puree (13-15° Brix), water, orange flower water, and salt were combined together (Part I), and then processed using high-shear mixing (e.g., blender, liquifier, etc.) until a smooth, homogeneous mixture was obtained. In some examples, the fruit purees may be combined in a ratio of the operator's discretion so that the total amount of sugar in the product ranges between 5-10% of the total weight of the liquid preparation. In some cases, the fruit purees may be fully or partially substituted in a 1:1 ratio with whole fruit. Popsicle molds were then filled with an amount of approximately 2.5-3 ounces by weight of mixture and were frozen (quiescent or non-quiescent) until the mixture was firm and reached a minimum internal temperature of −20° C.

Next, the strawberry puree, miracle berry powder and miraculin solution were combined (Part II), and high-shear mixing (e.g., blender, liquifier, etc.) was used to process the ingredients until a smooth, homogeneous mixture was obtained for dipping. The miracle berry powder (powdered berry from Synsepalum dulcificum) was purchased from Miracle Fruit Farm, FL.

Next, popsicles were removed from the molds and approximately 10-20% of the popsicle tip was dipped into the prepared miracle berry coating mixture and removed, so that a total of approximately 2-4 grams of the coating mixture was adhered to the popsicle. The miracle berry coating was allowed to freeze on to the surface of the pop, for approximately 10-20 seconds. Finally, the frozen popsicle was packaged and stored in a freezer kept at a minimum of −20° C. until use or for up to 1 year.

### Example 16. Strawberry Fruit Popsicle

In this example, a taste-modifying polypeptide comprising miraculin was formulated into a strawberry fruit popsicle.

TABLE 15
Formulation of Strawberry fruit popsicle (6 servings)
Ingredients

| | | | |
|---|---|---|---|
| Part I. | Strawberry Puree | 540 | g |
| | Water | 18 | g |
| | Lemon Juice | 42 | g |
| | Rose Water | 0.3 | g |
| | Salt | 0.45 | g |
| Part II. | Strawberry Puree | 18 | g |
| | Miracle berry powder | 15 | g |
| | Miraculin solution (0.5 mg/ml) | 2.4 | ml |

The following steps were implemented to make strawberry fruit popsicles comprising miraculin. First, the strawberry puree, water, lemon juice, rose water, and salt were mixed together (Part I), and then high-shear mixing (e.g., blender, liquifier, etc.) was used to process the ingredients until a smooth, homogeneous mixture was obtained. In some examples, the fruit purees may be substituted in a 1:1 ratio with whole fruit of the same variety. Depending on the sensory profile and Brix measurement of the fruit puree (or whole fruit), the ratio of fruit:water:lemon juice may be adjusted to meet product specifications. Next, popsicle molds were filled with an amount of approximately 2.5-3 ounces by weight and frozen (quiescent or non-quiescent) until the mixture was firm and reached a minimum internal temperature of −20° C.

Next, the strawberry puree, miracle berry powder and miraculin solution were mixed (Part II), and high-shear mixing (e.g., blender, liquifier, etc.) was used to process the ingredients until a smooth, homogeneous mixture was obtained for dipping. The miracle berry powder (powdered berry from Synsepalum dulcificum) was purchased from Miracle Fruit Farm, FL. Next, popsicles were removed from the molds and approximately 10-20% of the popsicle tip was dipped into prepared miracle berry coating mixture and removed, so that a total of approximately 2-4 grams of the coating mixture was adhered to the popsicle. The miracle berry coating was allowed to freeze on to the surface of the pop, for approximately 10-20 seconds. Finally, the frozen popsicles were packaged and stored in a freezer kept at a minimum of −20° C. until use or for up to 1 year.

### Example 17: Pineapple Fruit Popsicle

In this example, a taste-modifying polypeptide comprising miraculin was formulated into a pineapple fruit popsicle.

TABLE 16
Formulation of Pineapple fruit popsicle (6 servings)
Ingredients

| | | | |
|---|---|---|---|
| Part I. | Pineapple Puree | 510 | g |
| | Lime Juice | 60 | g |
| | Water | 30 | g |
| | Natural Flavors | 0.025-0.1 | g |
| | Salt | 0.45 | g |
| Part II. | Strawberry Puree | 18 | g |
| | Miracle berry powder | 15 | g |
| | Miraculin solution (0.5 mg/ml) | 2.4 | ml |

The following steps were implemented to make pineapple fruit popsicles comprising miraculin. First, the pineapple puree, water, lime juice, water, salt and natural flavors were mixed together (Part I), and then high-shear mixing (e.g., blender, liquifier, etc.) was used to process the ingredients until a smooth, homogeneous mixture was obtained. Next, popsicle molds were filled with an amount of approximately 2.5-3 ounces by weight and frozen (quiescent or non-quiescent) until the mixture was firm and reached a minimum internal temperature of −20° C.

Next, the strawberry puree, miracle berry powder and miraculin solution were mixed (Part II) and high-shear mixing (e.g., blender, liquifier, etc.) was used to process the ingredients until a smooth, homogeneous mixture was obtained for dipping. The miracle berry powder (powdered berry from Synsepalum dulcificum) was purchased from Miracle Fruit Farm, FL. Next, the popsicles were removed from the molds and approximately 10-20% of the popsicle tip was dipped into the prepared miracle berry coating mixture and removed, so that a total of approximately 2-4 grams of the coating mixture was adhered to the popsicle. The miracle berry coating was allowed to freeze on to the surface of the pop, for approximately 10-20 seconds. Finally, the frozen popsicles were packaged and stored in a freezer kept at a minimum of −20° C. until use or for up to 1 year.

### Example 18. Yogurt

In this example, a taste-modifying polypeptide comprising miraculin is formulated into yogurt.

TABLE 17
Miraculin in Plain yogurt

| Recipe No. | 1 | 2 | 3 | 4 |
|---|---|---|---|---|
| Miraculin (0.5 mg/ml) | 0.25 | 0.5 | 0.6 | 1 |
| Miraculin (mg) | 0.125 | 0.25 | 0.3 | 0.5 |
| Plain yogurt (g) | 30 | 30 | 30 | 30 |
| % w Miraculin | 0.0004 | 0.0008 | 0.0010 | 0.0017 |

In this example, plain yogurt was Siggi's 4% milk fat plan yogurt.

TABLE 18
Miracle berry powder in Plain yogurt

| Recipe No. | 1 | 2 | 3 | 4 | 5 |
|---|---|---|---|---|---|
| Miracle berry powder (g) | 0.1 | 0.125 | 0.25 | 0.5 | 1 |
| Plain yogurt (g) | 30 | 30 | 30 | 30 | 30 |
| % w Miracle berry powder | 0.33 | 0.42 | 0.83 | 1.67 | 3.33 |

| Formulation | Ingredient | Amount | |
|---|---|---|---|
| 1 | YC-470 pellets | 2 | g |
| | 2% milk | 1600 | ml |
| 2 | YoFlex ® Mild 2.0 | 2 | g |
| | 2% milk | 1600 | ml |
| 3 | Yo-Flex ® YC-X11 | 2 | g |
| | 2% milk | 1600 | ml |

Procedure:

1600 ml of milk was heated to 195° F. in a pot over medium high heat. The milk was stirred frequently to prevent the milk from burning. The milk was removed from the heat and the milk was allowed to cool to 110° F. To make the concentrated stock solution of culture, tempered milk in 4 oz. sterile cups was inoculated by adding 2 g of either YC-470, Yo-Flex Mild 2.0, or Yo-Flex YC-X11 to 100 ml of warm milk. The mixture was agitated for 10-15 minutes to homogenize. The rest of the milk (1500 ml) was inoculated with 15 ml of stock solution for a 2% concentration and mixed thoroughly. The temperature and pH of the milk was measured before incubating at 104° F.-118° F. for 9 hours. At the end of the incubation period, a yogurt container was placed into an ice bath for rapid cooling before storing in the fridge. Yogurt formulations were compared in a side-by-side gustatory comparison. Yogurt formulation 41 (e.g., with YC-470) produced the best-tasting yogurt.

In this example, yogurt was Siggi's 4% milk fat plain yogurt. Miracle berry powder was purchased from Miracle Fruit Farm, FL.

### Example 19. Miraculin Spray

TABLE 19

| No. of Spray | 2 | 4 | 6 |
|---|---|---|---|
| Volume (ml) | 0.125 | 0.25 | 0.375 |
| Miraculin solution (mg) | 0.075 | 0.15 | 0.225 |
| % weight | 0.06 | 0.06 | 0.06 |

1) 1 ml spray contains 16 sprays;

2) Concentration of Miraculin: 0.6 mg/ml.

### Example 20. Miraculin Gummy Candy

TABLE 20

| Serving size | Recipe 1 | Recipe 2 | Recipe 3 |
|---|---|---|---|
| Miraculin (mg) | 0.12 | 0.18 | 0.25 |
| Miracle berry powder (g) | 0.256 | 0.256 | 0.256 |
| Gummy weight (g) | 2 | 2 | 2 |
| % w (Miraculin) | 0.0060 | 0.0090 | 0.0125 |
| % w (Miracle berry) | 12.8 | 12.8 | 12.8 |

Each gummy weight is ˜1 g, and service size is 2.

### Example 21. Miraculin Popsicle

TABLE 21
Miracle berry powder in popsicle

| Recipe No. | 1 | 2 | 3 | 4 |
|---|---|---|---|---|
| Miracle berry powder (g) | 0.125 | 0.25 | 0.5 | 1 |
| Ice pop (g) | 100 | 100 | 100 | 100 |
| % w Miracle berry powder | 0.125 | 0.25 | 0.5 | 1 |

TABLE 22
Miraculin in popsicle

| Recipe No. | 1 | 2 | 3 |
|---|---|---|---|
| Miraculin (mg) | 0.1 | 0.15 | 0.2 |
| Ice pop (g) | 100 | 100 | 100 |
| % w Miraculin | 0.00010 | 0.00015 | 0.00020 |

TABLE 23
Miracle berry powder and miraculin in popsicle

| Recipe No. | 1 | 2 | 3 |
|---|---|---|---|
| Miraculin (mg) | 0.1 | 0.15 | 0.2 |
| Miracle berry powder (g) | 0.25 | 0.25 | 0.25 |
| Ice pop (g) | 100 | 100 | 100 |
| % w Miraculin | 0.00010 | 0.00015 | 0.00020 |
| % w Miracle berry powder | 0.25 | 0.25 | 0.25 |

### Example 22. Oral Thin Film

TABLE 24

| | | | Net | | Net | |
|---|---|---|---|---|---|---|
| | | Solution | weight | After dry | weight | |
| No. | Tray | (g) | (g) | (g) | (g) | Film |
| 1 | 11.942 | 14.642 | 2.70 | 12.093 | 0.151 | 0.531 |
| 2 | 11.836 | 14.648 | 2.81 | 12.187 | 0.351 | 0.442 |
| 3 | 11.754 | 14.632 | 2.88 | 12.218 | 0.464 | 0.485 |
| 4 | 12.023 | 14.643 | 2.62 | 12.221 | 0.198 | 0.533 |
| 5 | 11.663 | 14.327 | 2.66 | 12.184 | 0.521 | 0.481 |
| 6 | 11.704 | 14.33 | 2.63 | 12.372 | 0.668 | 0.563 |
| 7 | 11.54 | 14.283 | 2.74 | 12.048 | 0.508 | 0.429 |
| 8 | 11.67 | 14.308 | 2.64 | 12.186 | 0.516 | 0.465 |
| 9 | 11.78 | 14.066 | 2.29 | 12.279 | 0.499 | 0.489 |
| 10 | 11.698 | 14.047 | 2.35 | 12.147 | 0.449 | 0.512 |
| 11 | 11.67 | 14.037 | 2.37 | 12.152 | 0.482 | 0.506 |
| 12 | 11.404 | 13.791 | 2.39 | 11.951 | 0.547 | 0.575 |
| Sum | | | 31.07 | | 5.35 | 6.011 |
| Miraculin | 28.8 | mg | | | | |
| (mg) | | | | | | |
| % w | | | | | 0.54% | 0.48% |
| Miraculin | | | | | | |

### Example 23. Fruit Gel

TABLE 25

| | Amount for |
|---|---|
| Ingredients | 24 serving |
| Pure Miracle Fruit Powder (g) | 9 |
| Cranberry juice (ml) | 140 |
| Unflavored gelatine (g) | 18 |
| Blended solution of citric acid and ascorbic acid (ml) | 15 |
| H₂O (ml) | 155 |
| Fruit gel (g) | 14.04 |
| Pure Miracle Fruit Powder (g) | 0.38 |
| % weight of Miracle berry powder | 2.67 |

### Example 24. Fruit Bites

TABLE 26

| | Amount for |
|---|---|
| Ingredients | 8 serving |
| Tapioca Starch (g) | 1.39 |
| Hydroxypropyl methylcellulose (HPMC, 2%) (ml) | 5 |
| Polysorbate 20 (Food grade) (ml) | 5 |
| Blended solution of citric acid and ascorbic acid (ml) | 5 |
| Pure Miracle Fruit Powder (g) | 5 |
| Vegetable glycerin (4%) (ml) | 8 |
| H₂O (ml) | 22 |

### Example 25. Soda

TABLE 27
Soda Formulation (Serving Size: 2 500 mL bottles)

| Ingredient | Amount | |
|---|---|---|
| Lime oil | 500 | μl |
| Water | 1000 | ml |
| Citric acid | 4.4 | g |
| Miraculin (0.6 mg/ml) | 1 | ml |

1. Add the lime oil and citric acid to the water and agitate to dissolve.

2. Carbonate water using the soda stream machine for a minute or until sufficiently carbonated.

3. Add 1 ml of miraculin solution to the bottle cap to be ingested in the first sip.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-176. (canceled)

177. A composition comprising:
a) a yogurt product; and
b) a miraculin polypeptide or functional fragment thereof in a separate formulation that is to be consumed with the yogurt product.

178. The composition of claim 177, wherein the separate formulation is a layer overlaying the yogurt product.

179. The composition of claim 177, wherein the miraculin polypeptide or functional fragment thereof is present at a concentration from about 0.100 mg to about 0.500 mg.

180. The composition of claim 177, wherein the miraculin polypeptide or functional fragment thereof is purified from a plant.

181. The composition of claim 180, wherein the plant is Synsepalum dulcificum.

182. The composition of claim 177, wherein the miraculin polypeptide or functional fragment thereof is present in the composition at a concentration of about 0.0001% w/w to about 0.0050% w/w.

183. The composition of claim 177, wherein the miraculin polypeptide or functional fragment thereof is provided as a whole berry powder.

184. The composition of claim 177, wherein the miraculin polypeptide or functional fragment thereof is a recombinant miraculin polypeptide.

185. The composition of claim 184, wherein the recombinant miraculin polypeptide is recombinantly produced in a yeast cell.

186. The composition of claim 185, wherein the yeast cell is of the species Pichia pastoris.

187. The composition of claim 177, wherein the composition contains less sugar than an equivalent composition not comprising the miraculin polypeptide or functional fragment thereof.

188. The composition of claim 177, wherein the composition comprises at least 50% less sugar than an equivalent composition not comprising the miraculin polypeptide or functional fragment thereof.

189. The composition of claim 187, wherein the miraculin polypeptide or functional fragment thereof has been replaced with sugar in the equivalent composition.
